(19) United States
(12) Patent Application Publication
(10) Pub. No.: US 2026/0237516 A1
(43) Pub. Date: Aug. 13, 2026

(54) METHODS USEFUL FOR ASSIGNING LIKELIHOOD OF GRADE GROUP >2 PROSTATE CANCER

(71) Applicant: The Regents of the University of Michigan, Ann Arbor, MI (US)

(72) Inventors: Lanbo Xiao, Ann Arbor, MI (US); Yuping Zhang, Ann Arbor, MI (US); Jeffrey J. Tosoian, Ann Arbor, MI (US); Arul M. Chinnaiyan, Ann Arbor, MI (US); Jacob Meyers, Ann Arbor, MI (US)

(21) Appl. No.: 19/181,899

(22) Filed: Apr. 17, 2025

Related U.S. Application Data
(60) Provisional application No. 63/635,804, filed on Apr. 18, 2024.

(51) Int. Cl. G16H 50/30; G16H 50/20

(52) U.S. Cl. CPC G16H 50/30; G16H 50/20

(57) ABSTRACT

Provided herein are methods useful for assessing risk of Grade Group (GG)≥2 prostate cancer or assigning a subject's GG≥2 prostate cancer risk category based on expression levels of GG≥2 prostate cancer markers and improved algorithms or algorithm input coefficients.

### CROSS-REFERENCE TO RELATED APPLICATIONS

This application claims the benefit of and priority from U.S. Provisional Patent Application No. 63/635,804, filed Apr. 18, 2024, which is incorporated by reference herein in its entirety.

### STATEMENT REGARDING FEDERALLY SPONSORED RESEARCH OR DEVELOPMENT

This invention was made with government support under CA214170, CA186786, and 10 CA231996 awarded by the National Institutes of Health. The government has certain rights in the invention.

### REFERENCE TO AN ELECTRONIC SEQUENCE LISTING

The contents of the electronic sequence listing (LXDX-004-001WO.xml; Size 50,388 bytes; and Date of Creation: Apr. 3, 2025) are herein incorporated by reference in their entireties.

### FIELD OF THE INVENTION

Provided herein are methods useful for assessing risk of Grade Group (GG)≥2 prostate cancer or assigning a subject's GG≥2 prostate cancer risk category based on expression levels of GG≥2 prostate cancer markers and improved algorithms or algorithm coefficients.

### BACKGROUND OF THE INVENTION

Prostate cancer is the third most common urologic malignancy and can originate from the prostate parenchyma or urinary collecting system. Prostate cell carcinoma, arising from the prostate parenchyma, is the most common malignant prostate tumor associated with an incidence of 313,780 cases and approximately 35,770 deaths yearly in the United States. From the urinary collecting system, urothelial cell carcinoma is the most common malignancy representing approximately 10-15% of all prostate tumors. The overall incidence of malignant prostate tumors is increasing and currently is the third most common form of genitourinary cancer. Both malignant and benign prostate tumors are increasingly diagnosed in incidental fashion with the use of advanced cross-sectional imaging. Accurate diagnosis of benign versus malignant tumor types is lacking, leading to avoidable prostate biopsies, treatment or overtreatment. Furthermore, there are currently no diagnostic tests from urine that accurately assess risk of GG≥2 prostate cancer in prostate biopsy-naïve men.

There is a significant need for improved methods for assessing risk of GG≥2 prostate cancer, particularly a subject's risk of having GG≥2 prostate cancer that is too low to warrant a prostate biopsy of the subject, and improved algorithms or algorithm input coefficients that substantially improve the assessed risk's accuracy.

### SUMMARY OF THE INVENTION

Provided herein are methods for assigning a likelihood that a prostate biopsy of a subject would detect Grade Group (GG)≥2 prostate cancer in the subject, the methods comprising:
a) detecting an amount of expression of each of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample of the subject's urine;
b) normalizing the amount of expression of each of the genes to an amount of expression of a reference gene to provide a normalized target gene value for each of the genes;
c) multiplying each normalized target gene value by a corresponding gene algorithm coefficient to provide a gene log it value for each of the genes, wherein the corresponding gene algorithm coefficient is a corresponding gene algorithm coefficient of Table B or Table D;
d) summing 1) the gene log it values and 2) an algorithm intercept value to provide a sample log it value, wherein the algorithm intercept value is an algorithm intercept value of Table B or Table D;
e) multiplying the sample log it value by a calibration slope value to provide a product, and adding to the product a calibration intercept value to provide a calibrated log it value; and
f) obtaining a risk score comprising performing the equation

$$\frac{{e}^{calibrated logit value}}{1 + {e}^{calibrated logit value}}$$

wherein the risk score indicates the likelihood.

Further provided herein are methods for assigning a subject's Grade Group (GG)≥2 prostate cancer risk category, the methods comprising:
a) detecting an amount of expression of each of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample of the subject's urine;
b) normalizing the amount of expression of each of the genes to an amount of expression of a reference gene to provide a normalized target gene value for each of the genes;
c) multiplying each normalized target gene value by a corresponding gene algorithm coefficient to provide a gene log it value for each of the genes, wherein the corresponding gene algorithm coefficient is a corresponding gene algorithm coefficient of Table B or Table D;
d) summing 1) the gene log it values and 2) an algorithm intercept value to provide a sample log it value, wherein the algorithm intercept value is an algorithm intercept value of Table B or Table D;
e) multiplying the sample log it value by a calibration slope value to provide a product, and adding to the product a calibration intercept value to provide a calibrated log it value;
f) obtaining a risk score comprising performing the equation

$$\frac{{e}^{calibrated logit value}}{1 + {e}^{calibrated logit value}}$$

g) comparing the risk score to a predetermined risk threshold value; and
h) assigning the risk category based on whether the risk score is 1) less than or equal to the risk threshold value, or 2) higher than the threshold value.

### BRIEF DESCRIPTION OF THE DRAWINGS

FIG. 1 is a flow chart outlining risk score calculation from raw data for biomarker-only algorithms.

FIG. 2 is a flow chart outlining risk score calculation from biomarker+clinical factor or biomarker+clinical factor+prostate volume algorithms.

FIG. 3 is a flow chart depicting the number of subjects (n=318) among 1365 eligible subjects listed in the University of Michigan biospecimen database (IRB HUM00042749) found to have GG≥2 prostate cancer on biopsy.

FIG. 4A and FIG. 4B depict the nomination and selection of biomarkers for the multiplex qPCR panel (FIG. 4A) and selection of the gene panel for use in the algorithms (FIG. 4B).

FIG. 5 depicts a line graph demonstrating that algorithm calibration reflects agreement between the predicted outcomes from the algorithm and observed outcomes in the development cohort.

FIG. 6A and FIG. 6B depict receiver-operating characteristic (ROC) curves and their corresponding areas under the curve (AUC) for the cross-validated MPS2 (FIG. 6A) and MPS2+ (FIG. 6B) tests. Included are ROC curves of individual cross-validation folds (thin gray lines) and the mean ROC of all cross-validation folds for MPS2 (green) and MPS2+ (blue).

FIG. 7A and FIG. 7B depict box and dot plots illustrating the distribution of MPS2 (FIG. 7A) and MPS2+ (FIG. 7B) values in men who had a benign (negative) prostate biopsy (green), GG1 prostate cancer on biopsy (blue), and GG≥2 prostate cancer on biopsy (yellow) in the external validation cohort. P-values were ≤0.001 for pairwise comparisons of GG≥2 prostate cancer with negative prostate biopsy result and GG1 prostate cancer for both MPS2 algorithms.

FIG. 8 depicts receiver-operating characteristic curves and areas under the curve (AUC) for PSA (gray), PCPTrc (yellow), PHI (purple), dmx2 (pink), dmx3 (maroon), MPS (orange), MPS2 (green), and MPS2+ (blue) in the external validation cohort.

FIG. 9 depicts calibration curves for GG≥2 prostate cancer for MPS2 and MPS2+ in the external validation cohort.

FIG. 10A is a decision curve analysis (DCA) plot showing net clinical benefit of pre-prostate biopsy testing with PSA (gray), PCPTrc (yellow), PHI (purple), dmx2 (pink), dmx3 (maroon), MPS (orange), MPS2 (green), and MPS2+ (blue) as compared to risk threshold values (indicated as “threshold probabilities”) for “prostate biopsy all” (black) or “prostate biopsy none” (dark green).

FIG. 10B depicts a DCA plot illustrating the net reduction in prostate biopsies performed per 100 patients without missing a single diagnosis of GG≥2 prostate cancer based on pre-biopsy testing with PSA (gray), PCPTrc (yellow), PHI (purple), dmx2 (pink), dmx3 (maroon), MPS (orange), MPS2 (green), and MPS2+ (blue) as compared to risk threshold values (indicated as “threshold probabilities”) for biopsying all patients.

FIG. 11 depicts a calibration curve for GG>2 prostate cancer for alternative initial prostate biopsy algorithms iMPS2 (maroon) and iMPS2+ (orange) plotted with the MPS2 (green) and MPS2+ (blue) algorithms in the external validation cohort.

FIG. 12 is a heatmap illustrating differential expression of RNA-sequencing target genes in the biomarker discovery set. The color bar below the column-wise hierarchical clustering tree represents sample pathology. Normal samples, defined as those with no cancer, are shown in yellow (N=220), GG<prostate cancers in blue (N=71), and GG≥2 prostate cancers in red (N=484). Red, white, and blue cells in rows represent differential gene expression, calculated as log 2 (fold change in gene expression) divided by mean expression in benign samples. Blue represents decreased expression relative to normal, red represents increased expression relative to normal, and white represents no difference. The column-wise dendrogram represents hierarchical clustering among GG≥2 and GG<2 prostate cancer to normal samples.

FIG. 13 is a schematic block diagram of an example system that includes a compute device that can be used to implement methods or steps described herein, according to an embodiment.

### DEFINITIONS

To facilitate an understanding of the present disclosure, a number of terms and phrases are defined below:

As used herein, the term “subject” refers to a mammal having a prostate. In some embodiments, the subject is a human subject. In some embodiments, the human subject is a prostate biopsy-naïve subject, who has never had a prostate biopsy.

As used herein, “GG≥2 prostate cancer” means Grade Group ≥2 prostate cancer”. In some embodiments, the GG≥2 prostate cancer is GG≥3 prostate cancer. In some embodiments, the GG≥2 prostate cancer is GG≥4 prostate cancer. In some embodiments, the GG≥2 prostate cancer is GG5 prostate cancer.

As used herein, “GG<2 prostate cancer” means “Grade Group <2 prostate cancer”. In some embodiments, the GG<2 prostate cancer is GG 1 prostate cancer. In some embodiments, the GG<2 prostate cancer is no prostate cancer.

As used herein, a “risk score” is a predicted probability that a subject's prostate biopsy would be positive and detect GG≥2 prostate cancer in the subject. The risk score is based in part on the amount of expression of one or more genes described herein present in a sample from a subject. In some embodiments, the risk score is a numerical value ranging from 0% to 100%, i.e., 0%<risk score <100%. In some embodiments, the numerical value is expressed as a decimal number ranging from 0.00 to 1.00%, i.e., 0.00<risk score <1.00. In some embodiments, the risk score is a qualitative read-out of “low risk” or “increasing risk”. In some embodiments, “low risk” or “increasing risk” is relative to a predetermined risk threshold value.

As used herein, the term “about” means ±10% variation from an immediately following numerical value unless otherwise indicated. Where the term “about” is present immediately before a numerical value, the present disclosure also includes the specific numerical value itself, unless specifically stated otherwise.

As used herein, a “true positive” is a subject whose risk category is “increasing risk” based on the subject's risk score or likelihood, and whose prostate biopsy, performed the day on which the subject provided his urine sample, was positive for GG≥2 prostate cancer.

As used herein, a “true negative” is a subject whose risk category is “low risk” based on the subject's risk score or likelihood, and whose prostate biopsy, performed the day on which the subject provided his urine sample, was negative for GG≥2 prostate cancer.

As used herein, a “false positive” is a subject whose risk category is “increasing risk” based on the subject's risk score or likelihood, and whose prostate biopsy, performed the day on which the subject provided his urine sample, was negative for GG≥2 prostate cancer.

As used herein, a “false negative” is a subject whose risk category is “low risk” based on the subject's risk score or likelihood, and whose prostate biopsy, performed the day on which the subject provided his urine sample, was positive for GG≥2 prostate cancer.

As used herein, a “prostate biopsy-naïve” subject is a subject who has not had a prostate biopsy prior to providing a urine sample useful in the present methods.

As used herein, a “prostate biopsy-prior negative” subject is a subject who has had one or more prostate biopsies, none of which was positive for GG≥21 prostate cancer.

### DETAILED DESCRIPTION OF THE INVENTION

Provided herein are methods for assigning a likelihood that a prostate biopsy of a subject would detect Grade Group (GG)≥2 prostate cancer in the subject, the methods comprising:
a) detecting an amount of expression of each of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample of the subject's urine;
b) normalizing the amount of expression of each of the genes to an amount of expression of a reference gene to provide a normalized target gene value for each of the genes;
c) multiplying each normalized target gene value by a corresponding gene algorithm coefficient to provide a gene log it value for each of the genes;
d) summing 1) the gene log it values and 2) an algorithm intercept to provide a sample log it value;
e) multiplying the sample log it value by a calibration slope to provide a product, and adding to the product a calibration intercept to provide a calibrated log it value; and
f) obtaining a risk score comprising performing the equation

$$\frac{{e}^{calibrated logit value}}{1 + {e}^{calibrated logit value}}$$

wherein the risk score indicates the likelihood.

Accordingly, provided herein are methods for assigning a likelihood that a prostate biopsy of a subject would detect Grade Group (GG)≥2 prostate cancer in the subject, the methods comprising: a) detecting an amount of expression of each of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample of the subject's urine;
b) normalizing the amount of expression of each of the genes to an amount of expression of a reference gene to provide a normalized target gene value for each of the genes;
c) multiplying each normalized target gene value by a corresponding gene algorithm coefficient to provide a gene log it value for each of the genes, wherein the corresponding gene algorithm coefficient is a corresponding gene algorithm coefficient of Table B or Table D;
d) summing 1) the gene log it values and 2) an algorithm intercept value to provide a sample log it value, wherein the algorithm intercept value is an algorithm intercept value of Table B or Table D;
e) multiplying the sample log it value by a calibration slope value to provide a product, and adding to the product a calibration intercept value to provide a calibrated log it value; and
f) obtaining a risk score comprising performing the equation

$$\frac{{e}^{calibrated logit value}}{1 + {e}^{calibrated logit value}}$$

wherein the risk score indicates the likelihood.

Further provided herein are methods for assigning a subject's Grade Group (GG)≥2 prostate cancer risk category, the methods comprising:
a) detecting an amount of expression of each of one or more of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample of the subject's urine;
b) normalizing the amount of expression of each of the genes to an amount of expression of a reference gene to provide a normalized target gene value for each of the genes;
c) multiplying each normalized target gene value by a corresponding gene algorithm coefficient to provide a gene log it value for each of the genes, wherein the corresponding gene algorithm coefficient is a corresponding gene algorithm coefficient of Table B or Table D;
d) summing 1) the gene log it values and 2) an algorithm intercept value to provide a sample log it value, wherein the algorithm intercept value is an algorithm intercept value of Table B or Table D;
e) multiplying the sample log it value by a calibration slope value to provide a product, and adding to the product a calibration intercept value to provide a calibrated log it value;
f) obtaining a risk score comprising performing the equation

$$\frac{{e}^{calibrated logit value}}{1 + {e}^{calibrated logit value}}$$

g) comparing the risk score to a predetermined risk d value; and
h) assigning the risk category based on whether the risk score is 1) less than or equal to the risk threshold value, or 2) higher than the threshold value.

In some embodiments, the methods are useful for prognosing, diagnosing, or treating prostate cancer. Importantly, detection of PSA (prostate specific antigen), the conventional method for prognosis and/or diagnosis of prostate cancer, is not a necessary step of the methods described herein. PSA elevation identified during PSA screening leads to a high rate of invasive and unnecessary biopsies in men without cancer and frequent overdiagnosis of GG<2, indolent cancers (e.g., GG 1). Accordingly, in some embodiments, one or more of the present methods do not comprise detecting an amount of PSA, wherein the amount of PSA is present in the sample of the subject's urine. The present methods can provide more precise prognosis or diagnosis of prostate cancer and help identify those subjects that can benefit from early therapeutic intervention, while sparing those subjects with indolent disease from an invasive prostate biopsy.

In some embodiments, the methods further comprise performing a prostate biopsy of the subject. In some embodiments, the methods further comprise recommending to the subject or to a healthcare provider of the subject (e.g., via a compute device, such as compute device 801 of FIG. 13, used or accessible by the subject or the subject's healthcare provider) that the subject undergo a prostate biopsy. In some embodiments, the subject undergoes a prostate biopsy. In some embodiments, the subject undergoes a prostate biopsy, and the prostate biopsy indicates the subject has Grade Group ≥2 prostate cancer. In some embodiments, the subject undergoes a prostate biopsy, and the prostate biopsy indicates the subject does not have Grade Group ≥2 prostate cancer.

In some embodiments, the methods further comprise recommending to the subject or a health care provider of the subject that the subject does not undergo a prostate biopsy or that a prostate biopsy of the subject is avoidable.

In some embodiments, the methods do not comprise performing a prostate biopsy of the subject. In some embodiments, the methods further comprise informing the subject or the healthcare provider of the subject of the likelihood that a prostate biopsy of the subject would detect GG≥2 prostate cancer in the subject. In some embodiments, the subject does not undergo a prostate biopsy. In some embodiments, the subject does not undergo a prostate biopsy after the subject or a healthcare provider is informed of the likelihood that a prostate biopsy of the subject would detect GG≥2 prostate cancer in the subject. In some embodiments, the subject undergoes a prostate biopsy. In some embodiments, the subject undergoes a prostate biopsy after the subject or a healthcare provider is informed of the likelihood that a prostate biopsy of the subject would detect GG≥2 prostate cancer in the subject.

The methods described herein are useful to identify subjects with GG≥2 prostate cancer for treatment and allow those identified as not having GG≥2 prostate cancer to avoid a biopsy or unnecessary treatment and, accordingly, its associated side effects. The methods as provided herein are useful to reduce the number of avoidable prostate biopsies, sparing healthy subjects or subjects having GG<2 prostate cancer from a costly, invasive procedure.

### I. Detecting Amount of Gene Expression

The present methods comprise detecting an amount of expression of each of one or more (e.g., 1, 2, 3, 4, 5, 6, 7, 8, 9, 10, 11, 12, 13, 14, 15, 16, or all 17) of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample, e.g., a sample of the subject's urine. In some embodiments, also provided are methods for prognosis, diagnosis or treatment that comprise detecting an amount of expression of one or more (e.g., 1, 2, 3, 4, 5, 6, 7, 8, 9, 10, 11, 12, 13, 14, 15, 16, or all 17) of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. Illustrative, non-limiting methods are described herein.

### Genes for Detecting

In some embodiments, the amount of expression is the amount of mRNA or protein expressed by the genes.

In some embodiments, the methods described herein comprise detecting an amount of expression of a TMPRSS2-ERG gene. A TMPRSS2-ERG gene fusion overexpresses the transcription factor ERG, which is present in both early- and late-stage prostate cancer. Numerous variations of TMPRSS2-ERG fusions have been identified, with the most common comprising exon 1 of TMPRSS2 and exons 4-11 of ERG. In some embodiments, a TMPRSS2-ERG gene fusion comprises a fusion of the nucleotide sequences of Ensembl gene identifiers ENSG00000184012 and ENSG00000157554. In some embodiments, a TMPRSS2-ERG gene fusion comprises the nucleotide sequence of SEQ ID NO:1 or a variant thereof.

In some embodiments, the methods described herein comprise detecting an amount of expression of a SCHLAP1 gene. SCHLAP1 is a long noncoding RNA overexpressed in a subset of prostate cancers. SCHLAP1 antagonizes the genome-wide localization and regulatory functions of the SWI/SNF chromatin-modifying complex. In some embodiments, the SCHLAP1 gene comprises the nucleotide sequence provided by the HUGO Gene Nomenclature Committee (HGNC). In some embodiments, the HGNC identifier for SCHLAP1 is 48603. In some embodiments, the SCHLAP1 gene is located at chromosome position 2q31.3. In some embodiments, a SCHLAP1 gene comprises the nucleotide sequence of Ensembl gene identifier ENSG00000281131. In some embodiments, a SCHLAP1 gene comprises the nucleotide sequence of SEQ ID NO:2 or a variant thereof.

In some embodiments, the methods described herein comprise detecting an amount of expression of a OR51E2 gene. OR51E2 is an odorant receptor (OR) which represent the largest G protein-coupled receptor (GPCR) family in the human genome. Activation of human ORs can influence cell proliferation. Specifically, OR51E2 has been identified as being involved in the regulation of cell growth, migration and the invasiveness of melanocytes, melanoma cells, and prostate cancer cells. In some embodiments, the OR51E2 gene comprises the nucleotide sequence provided by HGNC. In some embodiments, the HGNC identifier for OR51E2 is 15195. In some embodiments, the OR51E2 gene is located at chromosome position 11p15.4. In some embodiments, an OR51E2 gene comprises the nucleotide sequence of Ensembl gene identifier ENSG00000167332. In some embodiments, an OR51E2 gene comprises the nucleotide sequence of SEQ ID NO:3 or a variant thereof.

In some embodiments, the methods described herein comprise detecting an amount of expression of an APOC1 gene. APOC1 is the smallest apolipoprotein and is a component of both triglyceride-rich lipoproteins and high-density lipoproteins. APOC1 is involved in various biological processes and is related to the progression of multiple diseases such as diabetic nephropathy, Alzheimer's disease, and glomerulosclerosis. Recent studies have shown APOC1 may be associated with the development of cancers, including breast cancer, pancreatic cancer, lung cancer, and prostate cancer. In some embodiments, the APOC1 gene comprises the nucleotide sequence provided by HGNC. In some embodiments, the HGNC identifier for APOC1 is 607. In some embodiments, the APOC1 gene is located at chromosome position 19q13.32. In some embodiments, an APOC1 gene comprises the nucleotide sequence of Ensembl gene identifier ENSG00000130208. In some embodiments, an APOC1 gene comprises the nucleotide sequence of SEQ ID NO:4 or a variant thereof.

In some embodiments, the methods described herein comprise detecting an amount of expression of a PCAT14 gene. PCAT14 is a long non-coding RNA that exhibits both cancer and lineage specificity. PCAT14 is transcriptionally regulated by androgen receptor (AR) and endogenous PCAT14 overexpression suppresses cell invasion. In some embodiments, the PCAT14 gene comprises the nucleotide sequence provided by HGNC. In some embodiments, the HGNC identifier for PCAT14 is 48977. In some embodiments, the PCAT14 gene is located at chromosome position 22q11.23. In some embodiments, a PCAT14 gene comprises the nucleotide sequence of Ensembl gene identifier ENSG00000280623. In some embodiments, a PCAT14 gene comprises the nucleotide sequence of SEQ ID NO:5 or a variant thereof.

In some embodiments, the methods described herein comprise detecting an amount of expression of a CAMKK2 gene. CAMKK2 is a direct target of the AR and regulation can vary across disease stages. CAMKK2 has been identified as a drive of prostate cancer progression. In some embodiments, the CAMKK2 gene comprises the nucleotide sequence provided by HGNC. In some embodiments, the HGNC identifier for CAMKK2 is 1470. In some embodiments, the CAMKK2 gene is located at chromosome position 12q24.31. In some embodiments, a CAMKK2 gene comprises the nucleotide sequence of Ensembl gene ENSG00000110931. In some embodiments, a CAMKK2 gene comprises the nucleotide sequence of SEQ ID NO:6 or a variant thereof.

In some embodiments, the methods described herein comprise detecting an amount of expression of a PCA3 gene. PCA3 is a non-coding gene associated with prostate cancer. In some embodiments, the PCA3 gene comprises the nucleotide sequence provided by HGNC. In some embodiments, the HGNC identifier for PCA3 is 8637. In some embodiments, the PCA3 gene is located at chromosome position 9q21.2. In some embodiments, a PCA3 gene comprises the nucleotide sequence of Ensembl gene identifier ENSG00000225937. In some embodiments, a PCA3 gene comprises the nucleotide sequence of SEQ ID NO:7 or a variant thereof.

In some embodiments, the methods described herein comprise detecting an amount of expression of an NKAIN1 gene. NKAIN1 is a sodium/potassium transporting ATPase. In some embodiments, the NKAIN1 gene comprises the nucleotide sequence provided by HGNC. In some embodiments, the HGNC identifier for NKAIN1 is 25743. In some embodiments, the NKAIN1 gene is located at chromosome position 1p35.2. In some embodiments, an NKAIN1 gene comprises the nucleotide sequence of Ensembl gene identifier ENSG00000084628. In some embodiments, an NKAIN1 gene comprises the nucleotide sequence of SEQ ID NO:8 or a variant thereof.

In some embodiments, the methods described herein comprise detecting an amount of expression of a B3GNT6 gene. B3GNT6 is a member of the O-GlcNAc transferase (OGT) family and is responsible for the production of the core 3 structure of O-glycans. In some embodiments, the B3GNT6 gene comprises the nucleotide sequence provided by HGNC. In some embodiments, the HGNC identifier for B3GNT6 is 24141. In some embodiments, the B3GNT6 gene is located at chromosome position 11q13.5. In some embodiments, a B3GNT6 gene comprises the nucleotide sequence of Ensembl gene identifier ENSG00000198488. In some embodiments, a B3GNT6 gene comprises the nucleotide sequence of SEQ ID NO:9 or a variant thereof.

In some embodiments, the methods described herein comprise detecting an amount of expression of a TFF3 gene. TFF3 is a trefoil factor, which are secreted peptides produced by normal intestinal mucosa. Members of the trefoil family are overexpressed in a variety of cancers and are associated with tumor invasion, resistance to apoptosis, and metastasis. In some embodiments, the TFF3 gene comprises the nucleotide sequence provided by HGNC. In some embodiments, the HGNC identifier for TFF3 is 11757. In some embodiments, the TFF3 gene is located at chromosome position 21q22.3. In some embodiments, a TFF3 gene comprises the nucleotide sequence of Ensembl gene identifier ENSG00000160180. In some embodiments, a TFF3 gene comprises the nucleotide sequence of SEQ ID NO:10 or a variant thereof.

In some embodiments, the methods described herein comprise detecting an amount of expression of a SPON2 gene. SPON2 belongs to the F-spondin family of secreted extracellular matrix proteins, and is deregulated in some tumors, including prostate cancer. In some embodiments, the SPON2 gene comprises the nucleotide sequence provided by HGNC. In some embodiments, the HGNC identifier for SPON2 is 11253. In some embodiments, the SPON2 gene is located at chromosome position 4p16.3. In some embodiments, a SPON2 gene comprises the nucleotide sequence of Ensembl gene identifier ENSG00000159674. In some embodiments, a SPON2 gene comprises the nucleotide sequence of SEQ ID NO:11 or a variant thereof.

In some embodiments, the methods described herein comprise detecting an amount of expression of a PCGEM1 gene. PCGEM1 is a long non-coding RNA that is a prostate-specific transcript. In some embodiments, the PCGEM1 gene comprises the nucleotide sequence provided by HGNC. In some embodiments, the HGNC identifier for PCGEM1 is 30145. In some embodiments, the PCGEM1 gene is located at chromosome position 2q32.3. In some embodiments, a PCGEM1 gene comprises the nucleotide sequence of Ensembl gene identifier ENSG00000227418. In some embodiments, a PCGEM1 gene comprises the nucleotide sequence of SEQ ID NO:12 or a variant thereof.

In some embodiments, the methods described herein comprise detecting an amount of expression of a TRGV9 gene. TRGV9 is encoded by the TRG locus that rearranges to encode a TCRγ chain containing 14 variable genes, of which only 6 are functional, including TRGV9. In some embodiments, the TRGV9 gene comprises the nucleotide sequence provided by HGNC. In some embodiments, the HGNC identifier for TRGV9 is 12295. In some embodiments, the TRGV9 gene is located at chromosome position 7p14.1. In some embodiments, a TRGV9 gene comprises the nucleotide sequence of Ensembl gene identifier ENSG00000211695. In some embodiments, a TRGV9 gene comprises the nucleotide sequence of SEQ ID NO:13 or a variant thereof.

In some embodiments, the methods described herein comprise detecting an amount of expression of a TMSB15A gene. TMSB15A is an isoform of human thymosin beta 15 which is an actin-binding protein. TMSB15A is expressed in normal human prostate and prostate cancer tissue. In some embodiments, the TMSB15A gene comprises the nucleotide sequence provided by HGNC. In some embodiments, the HGNC identifier for TMSB15A is 30744. In some embodiments, the TMSB15A gene is located at chromosome position Xq22.1. In some embodiments, a TMSB15A gene comprises the nucleotide sequence of Ensembl gene identifier ENSG00000158164. In some embodiments, a TMSB15A gene comprises the nucleotide sequence of SEQ ID NO:14 or a variant thereof.

In some embodiments, the methods described herein comprise detecting an amount of expression of an ERG gene. ERG is a transcriptional regulator overexpressed in prostate cancer. In some embodiments, the ERG gene comprises the nucleotide sequence provided by HGNC. In some embodiments, the HGNC identifier for ERG is 3446. In some embodiments, the ERG gene is located at chromosome position 21q22.2. In some embodiments, an ERG gene comprises the nucleotide sequence of Ensembl gene identifier ENSG00000157554. In some embodiments, an ERG gene comprises the nucleotide sequence of SEQ ID NO:15 or a variant thereof.

In some embodiments, the methods described herein comprise detecting an amount of expression of a KLK4 gene. KLK4 is a member of the kallikrein (KLK) family of highly conserved serine proteases that play key roles in a variety of physiological and pathological processes. KLKs are secreted proteins that have extracellular substrates and function. KLK4 is overexpressed in prostate cancer. In some embodiments, the KLK4 gene comprises the nucleotide sequence provided by HGNC. In some embodiments, the HGNC identifier for KLK4 is 6365. In some embodiments, the KLK4 gene is located at chromosome position 19q13.41. In some embodiments, a KLK4 gene comprises the nucleotide sequence of Ensembl gene identifier ENSG00000167749. In some embodiments, a KLK4 gene comprises the nucleotide sequence of SEQ ID NO:16 or a variant thereof.

In some embodiments, the methods described herein comprise detecting an amount of expression of a HOXC6 gene. HOXC6 is a homeobox (HOX) gene. HOX genes are involved in organ development and homeostasis and have been shown to be involved in normal prostate and prostate cancer development. HOXC6 is overexpressed in prostate cancer. In some embodiments, the HOXC6 gene comprises the nucleotide sequence provided by HGNC. In some embodiments, the HGNC identifier for HOXC6 is 5128. In some embodiments, the HOXC6 gene is located at chromosome position 12q 13.13. In some embodiments, a HOXC6 gene comprises the nucleotide sequence of Ensembl gene identifier ENSG00000197757. In some embodiments, a HOXC6 gene comprises the nucleotide sequence of SEQ ID NO: 17 or a variant thereof.

Illustrative nucleotide sequences of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 are provided in Table A.

TABLE A
Illustrative nucleotide sequences of genes of the disclosure.

| SEQ ID | | |
|---|---|---|
| NO | Gene | Sequence |
| 1 | TMPRSS2-ERG | TAGGCGCGAG CTAAGCAGGA GGCGGAGGCG GAGGCGGAGG |
| | | GCGAGGGGCG GGGAGCGCCG CCTGGAGCGC GGCAGGAAGC |
| | | CTTATCAGTT GTGAGTGAGG ACCAGTCGTT GTTTGAGTGT |
| | | GCCTACGGAA CGCCACACCT GGCTAAGACA GAGATGACCG |
| | | CGTCCTCCTC CAGCGACTAT GGACAGACTT CCAAGATGAG |
| | | CCCACGCGTC CCTCAGCAGG ATTGGCTGTC TCAACCCCCA |
| | | GCCAGGGTCA CCATCAAAAT GGAATGTAAC CCTAGCCAGG |
| | | TGAATGGCTC AAG |
| 2 | SCHLAP1 | GCTTTTATGA GCTGTAACAC TCACCGCGAA GGTCCGCAGC |
| | | TTCACTCCTG AAGCCAGCGA GACCACGAGC CTACTGGGAG |
| | | GAACGAACAA CTCCCGACGC GCCGCCTTAA GAGCTGTAAC |
| | | ACTCACCGCG AAGGTCTGCA GCTTCACTCC TGAGCCAGCG |
| | | AGACCACGAA CCCACCAGAA GGAAAAAACT CCGAACACAT |
| | | CTGAACATCA GAAGCAACAA ACTCCGGACA CGCCGCCTTT |
| | | AAGAACTGTA ACACTCACTG CGAGGGTCCG CGGCTTCATT |
| | | CTTGAAGTGA GTGAGACCAA GAACCCACCA GTTCTGGACA |
| | | CAATTTCAAG TCCTCAGGTG CCATCAATAT TCTGAAAATG |
| | | GCAGTGATTT TTATTCAACC TGTATAAGGC ACTTTCACCA |
| | | TGTACCTGGA AGCAACATCT ACATCTTTTT CAGTTTCTTC |
| | | TACGCCAGGT GTGTGCTTAG CTCCATGACA AAAGGTGACA |
| | | GCTTATTCTG CAGCACACAC ACATCATCAA AGTGGGAGGT |
| | | GGTGAGACTG GCACACTGAC AGTCTGTCCT AGCAGATTTC |
| | | AGCTCACACT GCAATCTAGA TGCTGGGGAC ACAAGGTCCA |
| | | CCTTCCAGGA ATATGGCCAT GACACCAGAA ATCACAAACA |
| | | TGATGAGAAT GGAATGACTG GGGAAGAAGT GCCAGATGCT |
| | | TCACTTGTAA ATGAAGACCC AGCCTCTGGG GATGCAGATA |
| | | CCACCTCCCT GAAGAAGCTG AATATCTGCA GATAAGTGGA |
| | | GTTCACCAAT GATGAGGAGC GGGATGGAGA AAGGAGGTAG |
| | | GGAGAGTCAT CCAAGGAACA TGAGCAACAT GTTAAAAGCC |
| | | AAGTGGTTTA ATTTCTGGAG ATGGTGAACC CAAGAGGCTC |
| | | TGCTGGGAGA CAACAAAAAT AATGAAGAAT TGAACCAGAG |
| | | TCCGGTGAAT ATCAGCACTG GGACCAGTTA GCAGAGGAAA |
| | | AGGAAAGAAT AAAAGCGAAA AGAATGAAGA GTCATATGAT |
| | | TACCAACTTT TCCTTTTTCA TATAAATTGA GTGTATATGG |
| | | GTCTGGAACA ACCTGAATTT CCATCAAGTC CTGGCTAACC |
| | | TCATTATGTC CTATGAATAT TTTTGACTAA TCCCACTTTA |
| | | CATTAATCTG TATTGTGAAT GTGGATATTG AATTATATTT |
| | | CTTTGTAATC CCATTATCCA AAATCCAGTT CAGAGACTAT |
| | | TAGTTACCAA TGTTCACTGT GAAGGAAAAA AAAAAAAAAA |
| | | AAGCTCAGAG GATAAACATG TGATATGGTT TGGCTGTGTC |
| | | CCCACCCAAA TATCATCTTG AATTGTAGCT CCCATAATTC |
| | | CCACGTGTTG TGGGAGGGAC CCGGTGGGAG ATAATTGTAT |
| | | CATGGGGGTG GTTCCCCCAT ACTATTCTCA TAGTAGTGAA |
| | | TAAGTCTCAC AAAATCTGAT GGTTTTATGA GGGAAAACCC |
| | | CTTTCACCTG GTTCTCATTC TCTTCTCTGG TCTGTCGTCA |
| | | TGTAAGACAT GCCTTTCACC TTCTCCACCA TGACTGTGAG |
| | | GCCTCCCCAG CCACGTGGAA CTGTGAGCCC ATTAAACCTC |
| | | TTTCACTTAT AAAT |
| 3 | OR51E2 | CTTCTGGGAA TCTCCACACC CTGAAGACAC AGTGAGTTAG |
| | | CACCACCACC AGGAATTGGC CTTTCAGCTC TGTGCCTGTC |
| | | TCCAGTCAGG CTGGAATAAG TCTCCTCATA TTTGCAAGCT |
| | | CGGCCCTCCC CTGGAATCTA AAGCCTCCTC AGCCTTCTGA |
| | | GTCAGCCTGA AAGGAACAGG CCGAACTGCT GTATGGGCTC |
| | | TACTGCCAGT GTGACCTCAC CCTCTCCAGT CACCCCTCCT |
| | | CAGTTCCAGC TATGAGTTCC TGCAACTTCA CACATGCCAC |
| | | CTTTGTGCTT ATTGGTATCC CAGGATTAGA GAAAGCCCAT |
| | | TTCTGGGTTG GCTTCCCCCT CCTTTCCATG TATGTAGTGG |
| | | CAATGTTTGG AAACTGCATC GTGGTCTTCA TCGTAAGGAC |
| | | GGAACGCAGC CTGCACGCTC CGATGTACCT CTTTCTCTGC |
| | | ATGCTTGCAG CCATTGACCT GGCCTTATCC ACATCCACCA |
| | | TGCCTAAGAT CCTTGCCCTT TTCTGGTTTG ATTCCCGAGA |
| | | GATTAGCTTT GAGGCCTGTC TTACCCAGAT GTTCTTTATT |
| | | CATGCCCTCT CAGCCATTGA ATCCACCATC CTGCTGGCCA |
| | | TGGCCTTTGA CCGTTATGTG GCCATCTGCC ACCCACTGCG |
| | | CCATGCTGCA GTGCTCAACA ATACAGTAAC AGCCCAGATT |
| | | GGCATCGTGG CTGTGGTCCG CGGATCCCTC TTTTTTTTCC |
| | | CACTGCCTCT GCTGATCAAG CGGCTGGCCT TCTGCCACTC |
| | | CAATGTCCTC TCGCACTCCT ATTGTGTCCA CCAGGATGTA |
| | | ATGAAGTTGG CCTATGCAGA CACTTTGCCC AATGTGGTAT |
| | | ATGGTCTTAC TGCCATTCTG CTGGTCATGG GCGTGGACGT |
| | | AATGTTCATC TCCTTGTCCT ATTTTCTGAT AATACGAACG |
| | | GTTCTGCAAC TGCCTTCCAA GTCAGAGCGG GCCAAGGCCT |
| | | TTGGAACCTG TGTGTCACAC ATTGGTGTGG TACTCGCCTT |
| | | CTATGTGCCA CTTATTGGCC TCTCAGTGGT ACACCGCTTT |
| | | GGAAACAGCC TTCATCCCAT TGTGCGTGTT GTCATGGGTG |
| | | ACATCTACCT GCTGCTGCCT CCTGTCATCA ATCCCATCAT |
| | | CTATGGTGCC AAAACCAAAC AGATCAGAAC ACGGGTGCTG |
| | | GCTATGTTCA AGATCAGCTG TGACAAGGAC TTGCAGGCTG |
| | | TGGGAGGCAA GTGACCCTTA ACACTACACT TCTCCTTATC |
| | | TTTATTGGCT TGATAAACAT AATTATTTCT AACACTAGCT |
| | | TATTTCCAGT TGCCCATAAG CACATCAGTA CTTTTCTCTG |
| | | GCTGGAATAG TAAACTAAAG TATGGTACAT CTACCTAAAG |
| | | GACTATTATG TGGAATAATA CATACTAATG AAGTATTACA |
| | | TGATTTAAAG ACTACAATAA AACCAAACAT GCTTATAACA |
| | | TTAAGAAAAA CAATAAAGAT ACATGATTGA AACCAAGTTG |
| | | AAAAATAGCA TATGCCTTGG AGGAAATGTG CTCAAATTAC |
| | | TAATGATTTA GTGTTGTCCC TACTTTCTCT CTCTTTTTTC |
| | | TTTCTTTTTT TTTTATTATG GTTAGCTGTC ACATACAACT |
| | | TTTTTTTTTT TTGAGATGGG GTCTCGCTCT GTCACCAGGC |
| | | TGGAGTGCAG TGGCGCGATC TCGGCTCACT GCAACCTCCA |
| | | CATCCCATGT TGAAGTAATT CTTCTGCCTC AGCCTCCCGA |
| | | GTAGCTGGGA CTAGAGGAAC GTGCCACCAT GACTGGCTAA |
| | | TTTTCTGTAT TTTTTAGTAG AGACAGAGTT TCACCATGTT |
| | | GGCCAGGATG GTCTCGATCT CCTGACCTTG TGATCCACCC |
| | | GCCTCAGCCT CCCAAAGTGT TGGGATTACA GGTGTGAACC |
| | | ACTGTGCCCG GCCTGTGTAC AACTTTTTAA ATAGGGAATA |
| | | TGATAGCTTC GCATGGTGGT GTGCACCTAT AGCCCCCACT |
| | | GCCTGGAAAG CTGAGGTGGG AGAATCGCTT GAGTCCAGGA |
| | | GTTTGAGGTT ACAGTGATCC ACGATCGTAC CACTACACTC |
| | | CAGCCTGGGC AACAGAGCAA GACCCTGTCT CAAAGCATAA |
| | | AATGGAATAA CATATCAAAT GAAACAGGGA AAATGAAGCT |
| | | GACAATTTAT GGAAGCCAGG GCTTGTCACA GTCTCTACTG |
| | | TTATTATGCA TTACCTGGGA ATTTATATAA GCCCTTAATA |
| | | ATAATGCCAA TGAACATCTC ATGTGTGCTC ACAATGTTCT |
| | | GGCACTATTA TAAGTGCTTC ACAGGTTTTA TGTGTTCTTC |
| | | GTAACTTTAT GGAGTAGGTA CCATTTGTGT CTCTTTATTA |
| | | TAAGTGAGAG AAATGAAGTT TATATTATCA AGGGGACTAA |
| | | AGTCACACGG CTTGTGGGCA CTGTGCCAAG ATTTAAAATT |
| | | AAATTTGATG GTTGAATACA GTTACTTAAT GACCATGTTA |
| | | TATTGCTTCC TGTGTAACAT CTGCCATTTA TTTCCTCAGC |
| | | TGTACAAATC CTCTGTTTTC TCTCTGTTAC ACACTAACAT |
| | | CAATGGCTTT GTACTTGTGA TGAGAGATAA CCTTGCCCTA |
| | | GTTGTGGGCA ACACATGCAG AATAATCCTG TTTTACAGCT |
| | | GCCTTTCGTG ATCTTATTGC TTGCTTTTTT CCAGATTCAG |
| | | GGAGAATGTT GTTGTCTATT TGTCTCTTAC ATCTCCTTGA |
| | | TCATGTCTTC ATTTTTTAAT GTGCTCTGTA CCTGTCAAAA |
| | | ATTTTGAATG TACACCACAT GCTATTGTCT GAACTTGAGT |
| | | ATAAGATAAA ATAAAATTTT ATTTTAAATT TT |
| 4 | APOC1 | AGGCGGTCAG GGGAAGGCTC AGGAGGAGGG AGATCAACAT |
| | | CAACCTGCCC CGCCCCCTCC CCAGCCTGAT AAAGGTCCTG |
| | | CGGGCAGGAC AGGACCTCCC AACCAAGCCC TCCAGCAAGG |
| | | ATTCAGAGTG CCCCTCCGGC CTCGCCATGA GGCTCTTCCT |
| | | GTCGCTCCCG GTCCTGGTGG TGGTTCTGTC GATCGTCTTG |
| | | GAAGGCCCAG CCCCAGCCCA GGGGACCCCA GACGTCTCCA |
| | | GTGCCTTGGA TAAGCTGAAG GAGTTTGGAA ACACACTGGA |
| | | GGACAAGGCT CGGGAACTCA TCAGCCGCAT CAAACAGAGT |
| | | GAACTTTCTG CCAAGATGCG GGAGTGGTTT TCAGAGACAT |
| | | TTCAGAAAGT GAAGGAGAAA CTCAAGATTG ACTCATGAGG |
| | | ACCTGAAGGG TGACATCCCA GGAGGGGCCT CTGAAATTTC |
| | | CCACACCCCA GCGCCTGTGC TGAGGACTCC CTCCATGTGG |
| | | CCCCAGGTGC CACCAATAAA AATCCTACAG AAAA |
| 5 | PCAT14 | GAGATACGGC CTCGTGGGAA GGGAAAGACC TGACCGTCCC |
| | | CCAGCCCGAC ACCCGTAAAG GGTCTGTGCT GAGGAGGATT |
| | | AGTAAAAGGG GAAGGCCTCT TGCAGTTGAG ATAAGAGGAA |
| | | GGCCTCCGTC TCCTGCATGT CCTTGGGAAT GGAATGTCTT |
| | | GGTGTAAAAC CCGATAGTAC ATTCCTTCTA TTCTGAGAGA |
| | | AGAAAACCAC CCTGTGGCTG GAGGGTGAAG GTACTCTACA |
| | | GTGTGGTCAT TGAGGACAAG TTGACGAGAG AGTCCCAAGT |
| | | ACGTCCACGG TCAGCCTTGC GACATTTAAA GTTCTACAAT |
| | | GAACTCACTG GAGATGCAAA GAAAAGTGTG GAGATGGAGA |
| | | CACCCCAATC GACTCGCCAG TCTACAGGTG TATCCAGCAG |
| | | CTCCAAAGAG ACAGCAACCA GCAAGAATGG GCCATAGTGA |
| | | CGATGGTGGT TTTGTCAAAA AGAAAAGGGG GGGATATGTA |
| | | AGGAAAAGAG AGATCAGACT TTCACTGTGT CTATGTAGAA |
| | | AAGGAAGACA TAAGAAACTC CATTTTGATC TGTACTAAGA |
| | | AAAATTGTTT TGCCTTGAGA TGCTGTTAAT CTGTAACTTT |
| | | AGCCCCAACC CTGTGCTCAC GGAAACATGT GCTGTAAGGT |
| | | TTAAGGGATC TAGGGCTGTG CAGGATGTAC CTTGTTAACA |
| | | ATATGTTTGC AGGCAGTATG TTTGGTAAAA GTCATCGCCA |
| | | TTCTCCATTC TCGATTAACC AGGGGCTCAA TGCACTGTGG |
| | | AAAGCCACAG GAACCTCTGC CCAAGAAAGC CTGGCTGTTG |
| | | TGGGAAGTCA GGGACCCCGA ATGGAGGGAC CAGCTGGTGC |
| | | TGCATCAGGA AACATAAATT GTGAAGATTT CTTGGACATT |
| | | TATCAGTTTC CAAAATTAAT ACTTTTATAA TTTCTTACAC |
| | | CTGTCTTACT TTAATCTCTT AATCCTGTTA TCTTTGTAAG |
| | | CTGAGGATAT ACGTCACCTC AGGACCACTA TTGTACAAAT |
| | | TGATTGTAAA ACATGTTCAC ATGTGTTTGA ACAATATGAA |
| | | ATCAGTGCAC CTTGAAAATG AACAGAATAA CAGTGATTTT |
| | | AGGGAACAAA GGAAGACAAC CATAAGGTCT GACTGCCTGA |
| | | GGGGTCGGGC AAAAAGCCAT ATTTTTCTTC TTGCAGAGAG |
| | | CCTATAAATG GACGTGCAAG TAGGAGAGAT ATTGCTAAAT |
| | | T |
| 6 | CAMKK2 | AGAGCAAGCT GAGCCGAGCC GAGCCGAGCT GGGGGCGCAG |
| | | AGCGCGGGAG GCGGCGGCGG CGCGGAGCCC AGGTGGCTCC |
| | | GCTGCCGGAT GGGAGTGCCC CAGTGTGCTG GATGAAGCTG |
| | | GCGCATGCAC CATGTCATCA TGTGTCTCTA GCCAGCCCAG |
| | | CAGCAACCGG GCCGCCCCCC AGGATGAGCT GGGGGGCAGG |
| | | GGCAGCAGCA GCAGCGAAAG CCAGAAGCCC TGTGAGGCCC |
| | | TGCGGGGCCT CTCATCCTTG AGCATCCACC TGGGCATGGA |
| | | GTCCTTCATT GTGGTCACCG AGTGTGAGCC GGGCTGTGCT |
| | | GTGGACCTCG GCTTGGCGCG GGACCGGCCC CTGGAGGCCG |
| | | ATGGCCAAGA GGTCCCCCTT GACACCTCCG GGTCCCAGGC |
| | | CCGGCCCCAC CTCTCCGGTC GCAAGCTGTC TCTGCAAGAG |
| | | CGGTCCCAGG GTGGGCTGGC AGCCGGTGGC AGCCTGGACA |
| | | TGAACGGACG CTGCATCTGC CCGTCCCTGC CCTACTCACC |
| | | CGTCAGCTCC CCGCAGTCCT CGCCTCGGCT GCCCCGGCGG |
| | | CCGACAGTGG AGTCTCACCA CGTCTCCATC ACGGGTATGC |
| | | AGGACTGTGT GCAGCTGAAT CAGTATACCC TGAAGGATGA |
| | | AATTGGAAAG GGCTCCTATG GTGTCGTCAA GTTGGCCTAC |
| | | AATGAAAATG ACAATACCTA CTATGCAATG AAGGTGCTGT |
| | | CCAAAAAGAA GCTGATCCGG CAGGCCGGCT TTCCACGTCG |
| | | CCCTCCACCC CGAGGCACCC GGCCAGCTCC TGGAGGCTGC |
| | | ATCCAGCCCA GGGGCCCCAT TGAGCAGGTG TACCAGGAAA |
| | | TTGCCATCCT CAAGAAGCTG GACCACCCCA ATGTGGTGAA |
| | | GCTGGTGGAG GTCCTGGATG ACCCCAATGA GGACCATCTG |
| | | TACATGGTGT TCGAACTGGT CAACCAAGGG CCCGTGATGG |
| | | AAGTGCCCAC CCTCAAACCA CTCTCTGAAG ACCAGGCCCG |
| | | TTTCTACTTC CAGGATCTGA TCAAAGGCAT CGAGTACTTA |
| | | CACTACCAGA AGATCATCCA CCGTGACATC AAACCTTCCA |
| | | ACCTCCTGGT CGGAGAAGAT GGGCACATCA AGATCGCTGA |
| | | CTTTGGTGTG AGCAATGAAT TCAAGGGCAG TGACGCGCTC |
| | | CTCTCCAACA CCGTGGGCAC GCCCGCCTTC ATGGCACCCG |
| | | AGTCGCTCTC TGAGACCCGC AAGATCTTCT CTGGGAAGGC |
| | | CTTGGATGTT TGGGCCATGG GTGTGACACT ATACTGCTTT |
| | | GTCTTTGGCC AGTGCCCATT CATGGACGAG CGGATCATGT |
| | | GTTTACACAG TAAGATCAAG AGTCAGGCCC TGGAATTTCC |
| | | AGACCAGCCC GACATAGCTG AGGACTTGAA GGACCTGATC |
| | | ACCCGTATGC TGGACAAGAA CCCCGAGTCG AGGATCGTGG |
| | | TGCCGGAAAT CAAGCTGCAC CCCTGGGTCA CGAGGCATGG |
| | | GGCGGAGCCG TTGCCGTCGG AGGATGAGAA CTGCACGCTG |
| | | GTCGAAGTGA CTGAAGAGGA GGTCGAGAAC TCAGTCAAAC |
| | | ACATTCCCAG CTTGGCAACC GTGATCCTGG TGAAGACCAT |
| | | GATACGTAAA CGCTCCTTTG GGAACCCATT CGAGGGCAGC |
| | | CGGCGGGAGG AACGCTCACT GTCAGCGCCT GGAAACTTGC |
| | | TCACCAAAAA ACCAACCAGG GAATGTGAGT CCCTGTCTGA |
| | | GCTCAAGGAA GCAAGGCAGC GAAGACAACC TCCAGGGCAC |
| | | CGACCCGCCC CCCGTGGGGG AGGAGGAAGT GCTCTTGTGA |
| | | GAGGCAGTCC CTGCGTGGAA AGTTGCTGGG CCCCCGCCCC |
| | | CGGCTCCCCC GCACGCATGC ATCCACTGCG GCCGGAGGAG |
| | | GCCATGGAGC CCGAGTAGCT GCCTGGATCG CTCGACCTCG |
| | | CATGCGCGCC GCGTCGCCTC TGGGGGGCTG CTGCACCGCG |
| | | TTTCCATAGC AGCATGTCCT ACGGAAACCC AGCACGTGTG |
| | | TAGAGCCTCG ATCGTCATCT CTGGTTATTT GTTTTTTCCT |
| | | TTGTTGTTTT AAAGGGGACA AAAAAAAAAA AAGGACTTGA |
| | | CTCCATGACG TCGACCGTGG CCGCTGGCTG GCTGGACAGG |
| | | CGGGTGTGAG GAGTTGCAGA CCCAAACCCA CGTGCATTTT |
| | | GGGACAATTG CTTTTTAAAA CGTTTTTATG CCAAAAATCC |
| | | TTCATTGTGA TTTTCAGAAC CACGTCAGAT ATACCAAGTG |
| | | ACTGTGTGTG GGGTTTGACA ACTGTGGAAA GGCGAGCAGA |
| | | AAACTCCGGC GGTCTGAGGC CATGGAGGTG GTTGCTGCAT |
| | | TTGAGAGGGA GTAGGGGGCT AGATGTGGCT CCTAGTGCAA |
| | | ACCGGAAACC ATGGCACCTT CCAGAGCCGT GGTCTCAAGG |
| | | AGTCAGAGCA GGGCTGGCCC TCAGTAGCTG CAGGGAGCTT |
| | | TGATGCAACT TATTTGTAAG AAGGATTTTT AAATTTTTTA |
| | | TGGGTAGAAT TGTAGTCAGG AAAACAGAAA GGGCTTGAAA |
| | | TTTAATAAGT GCTGCTGGAA GGGGATTTTC CAAGCCTGGA |
| | | AGGGTATTCA GCAGCTGTGG TGGGGAAACA TTTCTCCTGA |
| | | AAGACTGAAC GTGTTTCTTC ATGACAGCTG CTCAAAGCAG |
| | | GTTTCTGAGA TAGCTGACCG AGCTCTGGTA AATCTCTTTG |
| | | TCAAATTACG AAAACTTCAG GGTGAAATCC TATGCTTCCA |
| | | TGTACATTAC ATGGCTTAAG ATTAAACAAA AACATTTTTC |
| | | AAGTCTCTAA CTAGAGTGAA CTCTAGAGCA CAGTAGTTCA |
| | | GAAACTATTT AGAGCTTCCA GGATATATTT CACAGCTTCA |
| | | GGCATGTGAT CAGTTAGAGC CGATGAAACC TATGCCCGCC |
| | | TGTATATATA TTAGCAGCTT AGCTAGTTCA TAACCTGTAT |
| | | ATTCTAAAGA CTGCTAAGGT TTTGTTTTCA TTTTAAATCC |
| | | TAGCTGATTG TTGTGGTCAA TGAAATACCC AGTTTCTGGA |
| | | GGGCCAGGTG GGAAATGCTT TCACTGGACC AACACACAAA |
| | | TGATCATCCT GAGGATCTGA GCTTCCCTAG ACTCCACACA |
| | | ATAACCTTGG GGCACCCTTT TAGAGAAGAC TGTTGAAACC |
| | | CACAGCACTC GTTGGGGTAT GAGGAAACCA GGGCTTGGCA |
| | | CAGGAAGTTC CCCTTTGTAG CTAAAAGTCC AGAAAGAAAG |
| | | GGTTCATCTT TTTGACTTCC AACTGATATT GGGAAGTTTG |
| | | GTTGAGGTTC AAGTGTGACT CCTTCCAGAG CCACAGGTAG |
| | | GGGAGTGTGA AGTTGAGGGG GAGGAAAGCT GGAAGGACTC |
| | | TGCCTTGGGA GATTCCCAGC TCTGCTTTCC AGCGCTTGGT |
| | | GGAATCTGGG CTGGGGAAAG ACGGCACCGG GAAACTCTGC |
| | | TTCCCCATTG TTTCCATCTG ATCAGCTGTG GTGTGAGGAC |
| | | TTCTCAGACA AAGGCAAGGC CTCGTGCCCC TGCCCAGCCC |
| | | ATTCATGGAG CCCTGGGCCT TCTTGGCTTC CATAGATCCT |
| | | AAGCTCTTGA CTGTAGTTTA GCCAGACTTG TTTTGCTATC |
| | | TTATAAGCAG TTCAGAATTA GGGAATGCTG GTTTTGAAGA |
| | | GCAAAGGACA GGTAGTCTAG AGAGGGTCGT CTGGCCTGCT |
| | | TGCTGGGTCT TTGTAACCCA GCACTTCCTC TTGCCCTCCT |
| | | GGCTTTATGT TTATGGGGAG AGGACTCAAT AGCTCCACCC |
| | | CTTCTGGCAC CAGATGGGGC TTGGTTAGTT TGCAATAAGC |
| | | ACCTTGCAGA GGTTAAAGCC AGCGGGTCCC TAGTCTTAGG |
| | | CCCAGCCTGC TTGTGTGGGC TCTGGCCTGG CCTGGTGGCT |
| | | GGCCCAGGGG GCAGCAGTGC TTAGAGCTTC TGCAGGGCTT |
| | | CTCTTGTTTA CACAGCTGCA TCAGACAATG CCATTTCTCC |
| | | CCACCACGGA ACCTTCCATC TAAGATTTCT TCCAGGGAAT |
| | | GCCAGCAATC AGGCAGCACC CAGCTGTGGG GGCAGTGGGG |
| | | TGGGGGAGAC CCACATTGAT GACTTTTTTT TTTTCTTTTA |
| | | ATGAAGAAAC ACCAAAGAAA GCTGTGGAAA GGACCTGCCC |
| | | CACATGAAAA GGATAAGCCA AGATGGCTGT AAACACAGAG |
| | | CATTTGAGCT GCCACTCTTG GAGCACATTG ATTTTTCAAA |
| | | AGCCAGCTCT GTCAGGAAAG GAGGTGCTGT TATGAGCAGC |
| | | TCTTCCAGTG GGCAAAGAGG ACGCCCATAA TTTCTTCCAT |
| | | TGCTAGCTCA TCTGTGGGAC CAATTTGGTG TAAGCAACCT |
| | | GTGGCCTGCA CTTGTGGCCT CGAAGGAAGC ACAAACCCTC |
| | | CATCCACTTC CCATTTCCTC TGCCCTTTTC CACCTCCCCC |
| | | TTCCATCCCA CCAGCTGCCA GTGGCTCCCA GAAAGCCTTA |
| | | TTGAGCCCCT TGTTGACACT TGGGGCTGCG GAGGCCTCTC |
| | | CCTACTGGTC TGGCCTTTCC TGAGAGGCAG GTCTTCCGTC |
| | | CTCAGAGCCT TTCTGGAACA AGGAGAATGC CTGTGCAGGT |
| | | GGACACACAG GCCTGGCCTG TCGCTCTCAC TTGTCTTCCA |
| | | GCGGGGAGCT TCACGTTGCC GAGTGGAAGA ACCATGACCT |
| | | CCACTTGCTT CCAAGGTGCT AGGGAAGTTT CAGGGTACGC |
| | | TGGTTCCCCT CTCCAGCTGG AGGCCGAGTT TCTGGGGACT |
| | | GCAGATTTTT CTACTCTGTG ATCGATTCAA TGCCCGATGC |
| | | TTCTGTTTCA TTCCCGACCC TTTCTACTAT GCATTTTCCT |
| | | TTTATCAGGT GTATAAAGTT AAATACTGTG TATTTATCAC |
| | | TAAAAAGTAC ATGAACTTAA GAGACAACTA AGCCTTTCGT |
| | | GTTTTTCCAC AGGTGTTTAA GCTTCTCTGT ACAGTTGAAA |
| | | TAAACAGACA GCAAAATGGT GCCAA |
| 7 | PCA3 | ACAGAAGAAA TAGCAAGTGC CGAGAAGCTG GCATCAGAAA |
| | | AACAGAGGGG AGATTTGTGT GGCTGCAGCC GAGGGAGACC |
| | | AGGAAGATCT GCATGGTGGG AAGGACCTGA TGATACAGAG |
| | | GTGAGAAATA AGAAAGGCTG CTGACTTTAC |
| | | CATCTGAGGC CACACATCTG CTGAAATGGA GATAATTAAC |
| | | ATCACTAGAA ACAGCAAGAT GACAATATAA TGTCTAAGTA |
| | | GTGACATGTT TTTGCACATT TCCAGCCCCT TTAAATATCC |
| | | ACACACACAG GAAGCACAAA AGGAAGCACA GAGATCCCTG |
| | | GGAGAAATGC CCGGCCGCCA TCTTGGGTCA TCGATGAGCC |
| | | TCGCCCTGTG CCTGGTCCCG CTTGTGAGGG AAGGACATTA |
| | | GAAAATGAAT TGATGTGTTC CTTAAAGGAT GGGCAGGAAA |
| | | ACAGATCCTG TTGTGGATAT TTATTTGAAC GGGATTACAG |
| | | ATTTGAAATG AAGTCACAAA GTGAGCATTA CCAATGAGAG |
| | | GAAAACAGAC GAGAAAATCT TGATGGCTTC ACAAGACATG |
| | | CAACAAACAA AATGGAATAC TGTGATGACA TGAGGCAGCC |
| | | AAGCTGGGGA GGAGATAACC ACGGGGCAGA GGGTCAGGAT |
| | | TCTGGCCCTG CTGCCTAAAC TGTGCGTTCA TAACCAAATC |
| | | ATTTCATATT TCTAACCCTC AAAACAAAGC TGTTGTAATA |
| | | TCTGATCTCT ACGGTTCCTT CTGGGCCCAA CATTCTCCAT |
| | | ATATCCAGCC ACACTCATTT TTAATATTTA GTTCCCAGAT |
| | | CTGTACTGTG ACCTTTCTAC ACTGTAGAAT AACATTACTC |
| | | ATTTTGTTCA AAGACCCTTC GTGTTGCTGC CTAATATGTA |
| | | GCTGACTGTT TTTCCTAAGG AGTGTTCTGG CCCAGGGGAT |
| | | CTGTGAACAG GCTGGGAAGC ATCTCAAGAT CTTTCCAGGG |
| | | TTATACTTAC TAGCACACAG CATGATCATT ACGGAGTGAA |
| | | TTATCTAATC AACATCATCC TCAGTGTCTT TGCCCATACT |
| | | GAAATTCATT TCCCACTTTT GTGCCCATTC TCAAGACCTC |
| | | AAAATGTCAT TCCATTAATA TCACAGGATT AACTTTTTTT |
| | | TTTAACCTGG AAGAATTCAA TGTTACATGC AGCTATGGGA |
| | | ATTTAATTAC ATATTTTGTT TTCCAGTGCA AAGATGACTA |
| | | AGTCCTTTAT CCCTCCCCTT TGTTTGATTT TTTTTCCAGT |
| | | ATAAAGTTAA AATGCTTAGC CTTGTACTGA GGCTGTATAC |
| | | AGCCACAGCC TCTCCCCATC CCTCCAGCCT TATCTGTCAT |
| | | CACCATCAAC CCCTCCCATG CACCTAAACA AAATCTAACT |
| | | TGTAATTCCT TGAACATGTC AGGCATACAT TATTCCTTCT |
| | | GCCTGAGAAG CTCTTCCTTG TCTCTTAAAT CTAGAATGAT |
| | | GTAAAGTTTT GAATAAGTTG ACTATCTTAC TTCATGCAAA |
| | | GAAGGGACAC ATATGAGATT CATCATCACA TGAGACAGCA |
| | | AATACTAAAA GTGTAATTTG ATTATAAGAG TTTAGATAAA |
| | | TATATGAAAT GCAAGAGCCA CAGAGGGAAT GTTTATGGGG |
| | | CACGTTTGTA AGCCTGGGAT GTGAAGCAAA GGCAGGGAAC |
| | | CTCATAGTAT CTTATATAAT ATACTTCATT TCTCTATCTC |
| | | TATCACAATA TCCAACAAGC TTTTCACAGA ATTCATGCAG |
| | | TGCAAATCCC CAAAGGTAAC CTTTATCCAT TTCATGGTGA |
| | | GTGCGCTTTA GAATTTTGGC AAATCATACT GGTCACTTAT |
| | | CTCAACTTTG AGATGTGTTT GTCCTTGTAG TTAATTGAAA |
| | | GAAATAGGGC ACTCTTGTGA GCCACTTTAG GGTTCACTCC |
| | | TGGCAATAAA GAATTTACAA AGAGCTACTC AGGACCAGTT |
| | | GTTAAGAGCT CTGTGTGTGT GTGTGTGTGT GTGAGTGTAC |
| | | ATGCCAAAGT GTGCCTCTCT CTCTTTGACC CATTATTTCA |
| | | GACTTAAAAA CAAGCATGTT TTCAAATGGC ACTATGAGCT |
| | | GCCAATGATG TATCACCACC ATATCTCATT ATTCTCCAGT |
| | | AAATGTGATA ATAATGTCAT CTGTTAACAT AAAAAAAGTT |
| | | TGACTTCACA AAAGCAGCTG GAAATGGACA ACCACAATAT |
| | | GCATAAATCT AACTCCTACC ATCAGCTACA CACTGCTTGA |
| | | CATATATTGT TAGAAGCACC TCGCATTTGT GGGTTCTCTT |
| | | AAGCAAAATA CTTGCATTAG GTCTCAGCTG GGGCTGTGCA |
| | | TCAGGCGGTT TGAGAAATAT TCAATTCTCA GCAGAAGCCA |
| | | GAATTTGAAT TCCCTCATCT TTTAGGAATC ATTTACCAGG |
| | | TTTGGAGAGG ATTCAGACAG CTCAGGTGCT TTCACTAATG |
| | | TCTCTGAACT TCTGTCCCTC TTTGTGTTCA TGGATAGTCC |
| | | AATAAATAAT GTTATCTTTG AACTGATGCT CATAGGAGAG |
| | | AATATAAGAA CTCTGAGTGA TATCAACATT AGGGATTCAA |
| | | AGAAATATTA GATTTAAGCT CACACTGGTC AAAAGGAACC |
| | | AAGATACAAA GAACTCTGAG CTGTCATCGT CCCCATCTCT |
| | | GTGAGCCACA ACCAACAGCA GGACCCAACG CATGTCTGAG |
| | | ATCCTTAAAT CAAGGAAACC AGTGTCATGA GTTGAATTCT |
| | | CCTATTATGG ATGCTAGCTT CTGGCCATCT CTGGCTCTCC |
| | | TCTTGACACA TATTAGCTTC TAGCCTTTGC TTCCACGACT |
| | | TTTATCTTTT CTCCAACACA TCGCTTACCA ATCCTCTCTC |
| | | TGCTCTGTTG CTTTGGACTT CCCCACAAGA ATTTCAACGA |
| | | CTCTCAAGTC TTTTCTTCCA TCCCCACCAC TAACCTGAAT |
| | | GCCTAGACCC TTATTTTTAT TAATTTCCAA TAGATGCTGC |
| | | CTATGGGCTA TATTGCTTTA GATGAACATT AGATATTTAA |
| | | AGCTCAAGAG GTTCAAAATC CAACTCATTA TCTTCTCTTT |
| | | CTTTCACCTC CCTGCTCCTC TCCCTATATT ACTGATTGCA |
| | | CTGAACAGCA TGGTCCCCAA TGTAGCCATG CAAATGAGAA |
| | | ACCCAGTGGC TCCTTGTGGT ACATGCATGC AAGACTGCTG |
| | | AAGCCAGAAG GATGACTGAT TACGCCTCAT GGGTGGAGGG |
| | | GACCACTCCT GGGCCTTCGT GATTGTCAGG AGCAAGACCT |
| | | GAGATGCTCC CTGCCTTCAG TGTCCTCTGC ATCTCCCCTT |
| | | TCTAATGAAG ATCCATAGAA TTTGCTACAT TTGAGAATTC |
| | | CAATTAGGAA CTCACATGTT TTATCTGCCC TATCAATTTT |
| | | TTAAACTTGC TGAAAATTAA GTTTTTTCAA AATCTGTCCT |
| | | TGTAAATTAC TTTTTCTTAC AGTGTCTTGG CATACTATAT |
| | | CAACTTTGAT TCTTTGTTAC AACTTTTCTT ACTCTTTTAT |
| | | CACCAAAGTG GCTTTTATTC TCTTTATTAT TATTATTTTC |
| | | TTTTACTACT ATATTACGTT GTTATTATTT TGTTCTCTAT |
| | | AGTATCAATT TATTTGATTT AGTTTCAATT TATTTTTATT |
| | | GCTGACTTTT AAAATAAGTG ATTCGGGGGG TGGGAGAACA |
| | | GGGGAGGGAG AGCATTAGGA CAAATACCTA ATGCATGTGG |
| | | GACTTAAAAC CTAGATGATG GGTTGATAGG TGCAGCAAAC |
| | | CACTATGGCA CACGTATACC TGTGTAACAA ACCTACACAT |
| | | TCTGCACATG TATCCCAGAA CGTAAAGTAA AATTTAAAAA |
| | | AAAGTGA |
| 8 | NKAIN1 | AGTGCTGCTC TGCGCTGCGC CGCGCTCGGG GCTCGCTCTC |
| | | CTTGCTCCGC GCTCCCCGCC AGCCGCCCCG GGGCAGGAGG |
| | | CGCGCCTGAC GGACGGCCCG CTAGACAAAG GAGGCGCGGC |
| | | TCGGCGGGGC CAGCGCGCGG ACGGACGGAC CATGGACTCG |
| | | GAGCGCGGGC GGCCGGCCCC AGCCTTGGGG ACCGGACACT |
| | | CCCGGGCCCG GCCCTAGGCG CCCGGCCCCG CCGCCCGGCG |
| | | CGCCCAGCGG GGAGGACGTG GAGCCCGCGC GGCGCGAGCA |
| | | GGCGGCGGCC GCGGAGCAAG AAGGGCGCCG CGGCGTGCGG |
| | | CCCGCGCAGC CCCCGGAGCC ATGGGCAAGT GCAGCGGGCG |
| | | CTGCACGCTG GTCGCCTTCT GCTGCCTGCA GCTGGTGGCT |
| | | GCGCTGGAGC GGCAGATCTT TGACTTCCTG GGCTACCAGT |
| | | GGGCTCCCAT CCTAGCCAAC TTCCTGCACA TCATGGCAGT |
| | | CATCCTGGGC ATCTTTGGCA CCGTGCAGTA CCGCTCCCGG |
| | | TACCTCATCC TGTATGCAGC CTGGCTGGTG CTCTGGGTTG |
| | | GCTGGAATGC ATTTATCATC TGCTTCTACT TGGAGGTTGG |
| | | ACAGCTGTCC CAGGACCGGG ACTTCATCAT GACCTTCAAC |
| | | ACATCCCTGC ACCGCTCCTG GTGGATGGAG AATGGGCCAG |
| | | GCTGCCTGGT GACACCTGTT CTGAACTCCC GCCTGGCTCT |
| | | GGAGGACCAC CATGTCATCT CTGTCACTGG CTGCCTGCTT |
| | | GACTACCCCT ACATTGAAGC CCTCAGCAGC GCCCTGCAGA |
| | | TCTTCCTGGC ACTGTTCGGC TTCGTGTTCG CCTGCTACGT |
| | | GAGCAAAGTG TTCCTGGAGG AGGAGGACAG CTTTGACTTC |
| | | ATCGGCGGCT TTGACTCCTA CGGATACCAG GCGCCCCAGA |
| | | AGACGTCGCA TTTACAGCTG CAGCCTCTGT ACACGTCGGG |
| | | GTAGCCTCTG CCCCGCGCCC ACCCCGGCGC CTCGCCCTGG |
| | | GCTGACCGCA GCTGCCGCGA GCTCGGGCCA AGGCGCAGGC |
| | | GTGTCCCCCT GGTGGCCCGC GCGCTCACTG CAGCCTGTGC |
| | | CCAACCCCGC GTCTGCATCT GGAGATGCGG ACTTGGACGT |
| | | GGACTTGGAC TTGGACTTGG ATTTGAGCTT GGCTCTTCGC |
| | | AGCCCGGACT TCGGAGGAGT GGGGCGGGGC GGGGGAGGGG |
| | | CACCACGGGT TTTTTGTTTT TTGTTTGTTT GTTTTTAATC |
| | | TCAGCCTTGG CGTGAGCTGG GGCCTTCCTC TCTTCTCCAG |
| | | CCTCTCCCTT TCACTCTTCA CCCAGCATCC TGCCCCCCTG |
| | | TCCAAAAACA GCAGGACATC AGACCCATCC CATCCCACCA |
| | | CACTCACTCA CCAGCTCTGG GGAAAGCTAC TGTGAACTAG |
| | | GAGCAGGATT CCTGGGTTCT AATCGCAGGT CCATCACTGA |
| | | CTGTGACGTC TAGCAAAGCC CTTGCCCTCT CTGAGCCTCG |
| | | GTTTCCGCAC CTCAAGTAAT TAATCCCTTA GCAAATGGAC |
| | | TCTTTTAGAC TTCTCATTTA ACTCAATTCC CTGAGCTAGA |
| | | CTGGGATTAA AATTCTCATT TTGCAGTACA TTAAAACTGA |
| | | GGCCCAGAGA TGTGATTTGC TTGAGGCCAC ACAGCTAGAT |
| | | TTTTGGTGGA AGTGGGCCTT GAACACAGTG TACTTTCTGC |
| | | AGTTTCTGAC TGTAAAACCC AGTGTCTGCT CTCTGAGTTC |
| | | CATTTCCAAG CCCCCCTCCA TCTTGGACCT ATGTGGTCTC |
| | | CACCATATTC ACACACCACC ACCACCACTT GCCAATGCCT |
| | | CTCTTAAAGC AATATACCCA TTCGTTCTCT TATTGGGAAC |
| | | TGGATGGATG AAGCCCCAAA TTCAGCCCCA CCCACAGAGA |
| | | AGCCTTCCTA CACTCAGCCT CTGTCCACCC TTGGCAAATC |
| | | TTTCAAGCTC TCTCCTCCAG GAAAGTGGGG CCCCAACTCA |
| | | GTCACTCCAC CCCCTTCCAG GTCCCTGAGG CTGGTTCTAC |
| | | TGTATCCCCA TCACCTCCAC AACTCCACTC ACCCCTGACG |
| | | GCTCCATCCA CCTCACCAGT TGGAAGGCTT GTGGTTTCAG |
| | | AGAGGAGCAA TGCTGGTCAG CGCTGCCCAG ACTCCAGTGT |
| | | TTACAGATCA CCAGCATTTA CAACCAATCC AATGGCCAGA |
| | | AGCCTCCTCT AACAAGCCCA GAAGGAGTTC TGAAGGGGCA |
| | | GATGGGGGTG TGAGTAGTCG GGGAGTCGGG ATTGCCAGCA |
| | | CCCTCACCCT TCCTTGGGGG CAAGTAGAGG TGAGAACACT |
| | | TTCCCCACCT CCCTCCACAG ACACTCCTGA GGACGCTGCA |
| | | TCCCACGCAC TGCCTGGTGC GTCCATAGAG AGAGGATCAG |
| | | GTCTCAGCAT TTCATCTGTG AAAGAGGCAT GGCCCTGGGT |
| | | TAGAAAGGAG GGCAGGAGAC ATGGAGGAAC TGGGGGGCAC |
| | | CCAGATGGTG CAGATGGTTT GCACACCTGA GCCTGTCTGT |
| | | GGTGACCATT CCGCTCCTCT CCCACTACCC TCCAATCTAT |
| | | CATTCCCTAC TCTCTAAGGC CAAAATATCC TGAGCAAGGC |
| | | TGGCAACCCC ACCCCACCAT CCCAAATGCA AGCAGCCAGG |
| | | CCCAGGAGTT CCTCTGGCCC CCACAGGCAT GGAGCTCCCA |
| | | GCTGGTGGGT ACAGCTTGAG AGGGGGGCAG CTCCCTCAGG |
| | | CTAAGCTACT GCCCTTCACT GGGCCAGCCC TGCCTCCAGC |
| | | CCTCACCTCT CTCACCCCAA CTCTCCCCCA AGCCCCTTTC |
| | | TACTCAACGG GTGTAGCCAC TGGTGCTTTG AAGCCTTTTG |
| | | TTTTTATAAG ATGGTTTTTG CAAGGGGACC AGGTTCTCTT |
| | | TTCACTGGGA CCTTGCAAGG AGGGGAGTGC TCTCCTGGTT |
| | | TCTGTGCAGG CGGGTTGATT AAAGATGGTG TTTTCTTCTC TA |
| 9 | B3GNT6 | AGTGTGTGAA GTAAAGGGAT TAAAGGCTAG TCTCAGGCTG |
| | | GGGATGGCTC CTGTCTATTT CTTCTCTCTC AGAGACTGCA |
| | | GATGGCTTTT CCCTGCCGCA GGTCCCTGAC TGCCAAGACT |
| | | CTGGCCTGCC TCCTGGTGGG CGTGAGTTTC TTAGCACTGC |
| | | AGCAGTGGTT CCTCCAGGCG CCAAGGTCCC CGCGGGAGGA |
| | | GAGGTCCCCG CAGGAGGAGA CGCCAGAGGG TCCCACCGAC |
| | | GCTCCCGCGG CTGACGAGCC GCCCTCGGAG CTCGTCCCCG |
| | | GGCCCCCGTG CGTGGCGAAC GCCTCGGCGA ACGCCACGGC |
| | | CGACTTCGAG CAGCTGCCCG CGCGCATCCA GGACTTCCTG |
| | | CGGTACCGCC ACTGCCGCCA CTTCCCGCTG CTTTGGGACG |
| | | CACCGGCCAA GTGCGCCGGC GGCCGAGGCG TGTTCCTGCT |
| | | CCTGGCGGTG AAGTCGGCGC CTGAGCACTA CGAGCGACGC |
| | | GAGCTCATCC GGCGCACGTG GGGGCAAGAG CGCAGCTACG |
| | | GCGGGCGGCC AGTGCGCCGC CTCTTTCTAT TGGGCACCCC |
| | | GGGCCCCGAG GACGAGGCGC GCGCGGAGCG GCTGGCGGAG |
| | | CTGGTGGCGC TGGAGGCGCG CGAGCACGGC GACGTGCTGC |
| | | AGTGGGCCTT CGCGGACACC TTCCTCAACC TCACGCTCAA |
| | | GCACCTGCAC TTGCTCGACT GGCTGGCTGC ACGCTGCCCG |
| | | CACGCGCGCT TTCTGCTCAG CGGCGACGAC GACGTGTTCG |
| | | TGCACACCGC CAACGTAGTC CGCTTCCTGC AGGCGCAGCC |
| | | ACCCGGCCGC CACCTGTTCT CCGGCCAGCT CATGGAGGGC |
| | | TCCGTGCCCA TCCGCGACAG CTGGAGCAAG TACTTCGTGC |
| | | CGCCGCAGCT CTTCCCCGGG TCCGCTTACC CGGTGTACTG |
| | | CAGCGGCGGC GGCTTCCTCC TGTCCGGCCC CACGGCCCGG |
| | | GCCCTGCGCG CGGCCGCCCG CCACACCCCG CTCTTCCCCA |
| | | TCGACGACGC CTACATGGGC ATGTGTCTGG AGCGCGCCGG |
| | | CCTGGCGCCC AGCGGCCACG AGGGCATCCG ACCCTTCGGC |
| | | GTGCAGCTGC CTGGCGCACA GCAGTCCTCC TTCGACCCCT |
| | | GCATGTACCG CGAGTTGCTG CTAGTGCACC GCTTCGCGCC |
| | | CTACGAGATG CTGCTCATGT GGAAGGCGCT GCACAGCCCC |
| | | GCGCTCAGCT GTGACCGGGG ACACCGGGTC TCCTGAGGCC |
| | | AGTTGGGCGG CTTCAGCCCC GGGCCTCCAA CCATGTCCAT |
| | | GCTGAGAAGG CAGCTTTCCC GCTCTGGGTA CCTTACGTCC |
| | | TGCCCAGCTC TGTGCACCTG AACCCCAGCT GCGCACTGAA |
| | | ATCAGCTGGG GTGGGGGGTG TGGAAAATGC CTACATCCTG |
| | | GCTCCATCTC CCGAAGTTTC GATTTGATTA GTCTGGGGTG |
| | | GACCCAGACA TGTTAAGTAT TTTTTAAGTT CCTCCAGTGA |
| | | TGCGAATGTG CAGCTAGGCC TGAGGACCAC TCGGCTAGAC |
| | | TATCTCTTCA TCCTCGCAAA GCCAGCTCCA CCGCCCTCTC |
| | | TGCAAGAATT CCGGGCCCCT CGCTCCCACA CTCGGGTCCT |
| | | CTTGAGCAGT GGAGCAAGGG AGACCTGGGA GCGTGGGAGC |
| | | CAGGATCAGC GCCCCCTGCC ATGTGCCTAC AAATGTCAGT |
| | | TGTGATTTCC ACTGTTTACA AGTGAGTGGA GCTGGAGCTG |
| | | GGCTGACAGT ATCAGGTGGA TCCCGCTTCC CCCTCCCCCA |
| | | AGAAGTCAGC CAACACGCAG CTGAGGCGCA TGTGGTGGCC |
| | | TTCTTCCCAC CACTACCCCA GTACACCGTG AGGTAGAAAT |
| | | CTTCACCGTG CAAAGTGGAA ACCAGAGGCC CGGTCAGACA |
| | | GTGACTAATC CAGGGCCGTG GCATTCCCAG ACAGCACACC |
| | | ACTGTGGTCC CCTCCACACT CACCCCAACC AAAGCTAATG |
| | | GCCTAGTTGG GTCCTGCCCG CCAATAATCA CCCCCACGGG |
| | | TCAGAGACAG GCTCCTTGCC GGGGTCTGGG CCTCAGGCTC |
| | | AGTGGGCCTT GGACAACCCA GCAGGGAGTT CCGGGGAGTC |
| | | CGAAGTGGAG AAAGGCTGGT GGGAACATGG AGGCCAGTGT |
| | | TGGGGAGCCT GTGGAGGCAG GTGTGTAGAA TTGTGTTCGG |
| | | GAGGTGGGGG ATCTGAGACC GAAGTGGACA GTGGTTAAGA |
| | | TTGTGGGGCC GGGCGAGGTG GCTCACGCCT GTAATCCCAG |
| | | CACTTTGGGA GGCTGAGGAG GTCGGATCAT GAGGTCAAGA |
| | | GTTCGAGACC AGCCTGGCCA ATATGGTGAA ACCCCGTCTC |
| | | TATTGGGAGT ACAAAAATTA GCCGGCCATA GTGGCTCGTG |
| | | CCTGTAATCT CAGCTATTTG GGAGGCTGAG GCAGGAGAAT |
| | | CACTTGAACC TGGGAGGCGG AGGTTGCAGT GAGCCGAGAT |
| | | CGTGCCACTG CACTCCAGCC TGGGCGACAG AGCAAGACTG |
| | | CATCTCAAAA AAAAAAAAAA AAA |
| 10 | TFF3 | GAGTCCTGAG CTGCGTCCCG GAGCCCACGG TGGTCATGGC |
| | | TGCCAGAGCG CTCTGCATGC TGGGGCTGGT CCTGGCCTTG |
| | | CTGTCCTCCA GCTCTGCTGA GGAGTACGTG GGCCTGTCTG |
| | | CAAACCAGTG TGCCGTGCCA GCCAAGGACA GGGTGGACTG |
| | | CGGCTACCCC CATGTCACCC CCAAGGAGTG CAACAACCGG |
| | | GGCTGCTGCT TTGACTCCAG GATCCCTGGA GTGCCTTGGT |
| | | GTTTCAAGCC CCTGCAGGAA GCAGAATGCA CCTTCTGAGG |
| | | CACCTCCAGC TGCCCCCGGC CGGGGGATGC GAGGCTCGGA |
| | | GCACCCTTGC CCGGCTGTGA TTGCTGCCAG GCACTGTTCA |
| | | TCTCAGCTTT TCTGTCCCTT TGCTCCCGGC AAGCGCTTCT |
| | | GCTGAAAGTT CATATCTGGA GCCTGATGTC TTAACGAATA |
| | | AAGGTCCCAT GCTCCACCCG AGGACAGTTC TTCGTGCCTG |
| | | AGACTTTCTG AGGTTGTGCT TTATTTCTGC TGCGTCGTGG |
| | | GAGAGGGCGG GAGGGTGTCA GGGGAGAGTC TGCCCAGGCC |
| | | TCAAGGGCAG GAAAAGACTC CCTAAGGAGC TGCAGTGCAT |
| | | GCAAGGATAT TTTGAATCCA GACTGGCACC CACGTCACAG |
| | | GAAAGCCTAG GAACACTGTA AGTGCCGCTT CCTCGGGAAA |
| | | GCAGAAAAAA TACATTTCAG GTAGAAGTTT TCAAAAATCA |
| | | CAAGTCTTTC TTGGTGAAGA CAGCAAGCCA ATAAAACTGT |
| | | CTTCCAAAGT GGTCCTTTAT TTCACAACCA CTCTCGCTAC |
| | | TGTTCAATAC TTGTACTATT CCTGGGTTTT GTTTCTTTGT |
| | | ACAGTAAACA TTATGAACAA ACAGGCA |
| 11 | SPON2 | ACCCGACCGC TGCCGGCCGC GCTCCCGCTG CTCCTGCCGG |
| | | GTGATGGAAA ACCCCAGCCC GGCCGCCGCC CTGGGCAAGG |
| | | CCCTCTGCGC TCTCCTCCTG GCCACTCTCG GCGCCGCCGG |
| | | CCAGCCTCTT GGGGGAGAGT CCATCTGTTC CGCCAGAGCC |
| | | CTGGCCAAAT ACAGCATCAC CTTCACGGGC AAGTGGAGCC |
| | | AGACGGCCTT CCCCAAGCAG TACCCCCTGT TCCGCCCCCC |
| | | TGCGCAGTGG TCTTCGCTGC TGGGGGCCGC GCATAGCTCC |
| | | GACTACAGCA TGTGGAGGAA GAACCAGTAC GTCAGTAACG |
| | | GGCTGCGCGA CTTTGCGGAG CGCGGCGAGG CCTGGGCGCT |
| | | GATGAAGGAG ATCGAGGCGG CGGGGGAGGC GCTGCAGAGC |
| | | GTGCACGCGG TGTTTTCGGC GCCCGCCGTC CCCAGCGGCA |
| | | CCGGGCAGAC GTCGGCGGAG CTGGAGGTGC AGCGCAGGCA |
| | | CTCGCTGGTC TCGTTTGTGG TGCGCATCGT GCCCAGCCCC |
| | | GACTGGTTCG TGGGCGTGGA CAGCCTGGAC CTGTGCGACG |
| | | GGGACCGTTG GCGGGAACAG GCGGCGCTGG ACCTGTACCC |
| | | CTACGACGCC GGGACGGACA GCGGCTTCAC CTTCTCCTCC |
| | | CCCAACTTCG CCACCATCCC GCAGGACACG GTGACCGAGA |
| | | TAACGTCCTC CTCTCCCAGC CACCCGGCCA ACTCCTTCTA |
| | | CTACCCGCGG CTGAAGGCCC TGCCTCCCAT CGCCAGGGTG |
| | | ACACTGGTGC GGCTGCGACA GAGCCCCAGG GCCTTCATCC |
| | | CTCCCGCCCC AGTCCTGCCC AGCAGGGACA ATGAGATTGT |
| | | AGACAGCGCC TCAGTTCCAG AAACGCCGCT GGACTGCGAG |
| | | GTCTCCCTGT GGTCGTCCTG GGGACTGTGC GGAGGCCACT |
| | | GTGGGAGGCT CGGGACCAAG AGCAGGACTC GCTACGTCCG |
| | | GGTCCAGCCC GCCAACAACG GGAGCCCCTG CCCCGAGCTC |
| | | GAAGAAGAGG CTGAGTGCGT CCCTGATAAC TGCGTCTAAG |
| | | ACCAGAGCCC CGCAGCCCCT GGGGCCCCCC GGAGCCATGG |
| | | GGTGTCGGGG GCTCCTGTGC AGGCTCATGC TGCAGGCGGC |
| | | CGAGGGCACA GGGGGTTTCG CGCTGCTCCT GACCGCGGTG |
| | | AGGCCGCGCC GACCATCTCT GCACTGAAGG GCCCTCTGGT |
| | | GGCCGGCACG GGCATTGGGA AACAGCCTCC TCCTTTCCCA |
| | | ACCTTGCTTC TTAGGGGCCC CCGTGTCCCG TCTGCTCTCA |
| | | GCCTCCTCCT CCTGCAGGAT AAAGTCATCC CCAAGGCTCC |
| | | AGCTACTCTA AATTATGTCT CCTTATAAGT TATTGCTGCT |
| | | CCAGGAGATT GTCCTTCATC GTCCAGGGGC CTGGCTCCCA |
| | | CGTGGTTGCA GATACCTCAG ACCTGGTGCT CTAGGCTGTG |
| | | CTGAGCCCAC TCTCCCGAGG GCGCATCCAA GCGGGGGCCA |
| | | CTTGAGAAGT GAATAAATGG GGCGGTTTCG GAAGCGTCAG |
| | | TGTTTCCATG TTATGGATCT CTCTGCGTTT GAATAAAGAC |
| | | TATCTCTGTT GCTCACAAA |
| 12 | PCGEM1 | AAGGCACTCT GGCACCCAGT TTTGGAACTG CAGTTTTAAA |
| | | AGTCATAAAT TGAATGAAAA TGATAGCAAA GGTGGAGGTT |
| | | TTTAAAGAGC TATTTATAGG TCCCTGGACA GCATCTTTTT |
| | | TCAATTAGGC AGCAACCTTT TTGCCCTATG CCGTAACCTG |
| | | TGTCTGCAAC TTCCTCTAAT TGGGAAATAG TTAAGCAGAT |
| | | TCATAGAGCT GAATGATAAA ATTGTACTAC GAGATGCACT |
| | | GGGACTCAAC GTGACCTTAT CAAGTGAGCA GGCTTGGTGC |
| | | ATTTGACACT TCATGATATC AGCCAAAGTG GAACTAAAAA |
| | | CAGCTCCTGG AAGAGGACTA TGACATCATC AGGTTGGGAG |
| | | TCTCCAGGGA CAGCGGACCC TTTGGAAAAG GACTAGAAAG |
| | | TGTGAAATCT ATTAGTCTTC GATATGAAAT TCTCTGTCTC |
| | | TGTAAAAGCA TTTCATATTT ACAAGACACA GGCCTACTCC |
| | | TAGGGCAGCA AAAAGTGGCA ACAGGCAAGC AGAGGGAAAA |
| | | GAGATCATGA GGCATTTCAG AGTGCACTGT CTTTTCATAT |
| | | ATTTCTCAAT GCCGTATGTT TGGTTTTATT TTGGCCAAGC |
| | | ATAACAATCT GCTCAAGAAA AAAAAATCTG GAGAAAACAA |
| | | AGGTGCCTTT GCCAATGTTA TGTTTCTTTT TGACAAGCCC |
| | | TGAGATTTCT GAGGGGAATT CACATAAATG GGATCAGGTC |
| | | ATTCATTTAC GTTGTGTGCA AATATGATTT AAAGATACAA |
| | | CCTTTGCAGA GAGCATGCTT TCCTAAGGGT AGGCACGTGG |
| | | AGGACTAAGG GTAAAGCATT CTTCAAGATC AGTTAATCAA |
| | | GAAAGGTGCT CTTTGCATTC TGAAATGCCC TTGTTGCAAA |
| | | TATTGGTTAT ATTGATTAAA TTTACACTTA ATGGAAACAA |
| | | CCTTTAACTT ACAGATGAAC AAACCCACAA AAGCAAAAAA |
| | | TCAAAAGCCC TACCTATGAT TTCATATTTT CTGTGTAACT |
| | | GGATTAAAGG ATTCCTGCTT GCTTTTGGGC ATAAATGATA |
| | | ATGGAATATT TCCAGGTATT GTTTAAAATG AGGGCCCATC |
| | | TACAAATTCT TAGCAATACT TTGGATAATT CTAAAATTCA |
| | | GCTGGACATT GTCTAATTGT TTTTTATATA CATCTTTGCT |
| | | AGAATTTCAA ATTTTAAGTA TGTGAATTTA GTTAATTAGC |
| | | TGTGCTGATC AATTCAAAAA CATTACTTTC CTAAATTTTA |
| | | GACTATGAAG GTCATAAATT CAACAAATAT ATCTACACAT |
| | | ACAATTATAG ATTGTTTTTC ATTATAATGT CTTCATCTTA |
| | | ACAGAATTGT CTTTGTGATT GTTTTTAGAA AACTGAGAGT |
| | | TTTAATTCAT AATTACTTGA TCAAAAAATT GTGGGAACAA |
| | | TCCAGCATTA ATTGTATGTG ATTGTTTTTA TGTACATAAG |
| | | GAGTCTTAAG CTTGGTGCCT TGAAGTCTTT TGTACTTAGT |
| | | CCCATGTTTA AAATTACTAC TTTATATCTA AAGCATTTAT |
| | | GTTTTTCAAT TCAATTTACA TGATGCTAAT TATGGCAATT |
| | | ATAACAAATA TTAAAGATTT CGAAATAGAA AAAAAAAAAA |
| | | AAA |
| 13 | TRGV9 | GTGAGGACAC CGCTTTACAA CGATGCAGGG GGCCCCATGT |
| | | CACCCTCACC CATGGGAAGT TTGACTTGGT GGACTCAGCC |
| | | AAGCCACAGA GGTCTAACGC TTCTCTGCGG TGATTTCAGG |
| | | CTGCCCTGGC AGAAAGCACA GTGCCTGCAG ACATGCTGTC |
| | | ACTGCTCCAC GCATCAACGC TGGCAGTCCT TGGGGCTCTG |
| | | TGTGTATATG GTGCAGGTCA CCTAGAGCAA CCTCAAATTT |
| | | CCAGTACTAA AACGCTGTCA AAAACAGCCC GCCTGGAATG |
| | | TGTGGTGTCT GGAATAACAA TTTCTGCAAC ATCTGTATAT |
| | | TGGTATCGAG AGAGACCTGG TGAAGTCATA CAGTTCCTGG |
| | | TGTCCATTTC ATATGACGGC ACTGTCAGAA AGGAATCCGG |
| | | CATTCCGTCA GGCAAATTTG AGGTGGATAG GATACCTGAA |
| | | ACGTCTACAT CCACTCTCAC CATTCACAAT GTAGAGAAAC |
| | | AGGACATAGC TACCTACTAC TGTGCCTTGT TGGAGGGAAA |
| | | TTATAAGAAA CTCTTTGGCA GTGGAACAAC ACTTGTTGTC |
| | | ACAGATAAAC AACTTGATGC AGATGTTTCC CCCAAGCCCA |
| | | CTATTTTTCT TCCTTCAATT GCTGAAACAA AGCTCCAGAA |
| | | GGCTGGAACA TACCTTTGTC TTCTTGAGAA ATTTTTCCCT |
| | | GATGTTATTA AGATACATTG GCAAGAAAAG AAGAGCAACA |
| | | CGATTCTGGG ATCCCAGGAG GGGAACACCA TGAAGACTAA |
| | | CGACACATAC ATGAAATTTA GCTGGTTAAC GGTGCCAGAA |
| | | AAGTCACTGG ACAAAGAACA CAGATGTATC GTCAGACATG |
| | | AGAATAATAA AAACGGAGTT GATCAAGAAA TTATCTTTCC |
| | | TCCAATAAAG ACAGATGTCA TCACAATGGA TCCCAAAGAC |
| | | AATTGTTCAA AAGATGCAAA TGATACACTA CTGCTGCAGC |
| | | TCACAAACAC CTCTGCATAT TACATGTACC TCCTCCTGCT |
| | | CCTCAAGAGT GTGGTCTATT TTGCCATCAT CACCTGCTGT |
| | | CTGCTTAGAA GAACGGCTTT CTGCTGCAAT GGAGAGAAAT |
| | | CATAACAGAC GGTGGCACAA GGAGGCCATC TTTTCCTCAT |
| | | CGGTTATTGT CCCTAGAAGC GTCTTCTGAG GATCTAGTTG |
| | | GGCTTTCTTT CTGGGTTTGG GCCATTTCAG TTCTCATGTG |
| | | TGTACTATTC TATCATTATT GTATAACGGT TTTCAAACCA |
| | | GTGGGCACAC AGAGAACCTC ACTCTGTAAT AACAATGAGG |
| | | AATAGCCACG GCGATCTCCA GCACCAATCT CTCCATGTTT |
| | | TCCACAGCTC CTCCAGCCAA CCCAAATAGC GCCTGCTATA |
| | | GTGTAGACAT CCTGCGGCTT CTAGCCTTGT CCCTCTCTTA |
| | | GTGTTCTTTA ATCAGATAAC TGCCTGGAAG CCTTTCATTT |
| | | TACACGCCCT GAAGCAGTCT TCTTTGCTAG TTGAATTATG |
| | | TGGTGTGTTT TTCCGTAATA AGCAAAATAA ATTTAAAAAA |
| | | ATGAAAAGTT |
| 14 | TMSB15A | AACGCTAACC TGGTCCGGAG CGAGTCTGGG TCTCAGCCCC |
| | | GCGAACAGCC TTTCACGAGT CTTCAAGCTT TCAGGCTATC |
| | | TTCTAGTCAA GATGAGTGAT AAGCCAGACT TGTCGGAAGT |
| | | GGAGAAGTTT GACAGGTCAA AACTGAAGAA AACTAATACT |
| | | GAAGAAAAAA ATACTCTTCC CTCAAAGGAA ACTATCCAGC |
| | | AAGAGAAAGA GTGTGTTCAA ACATCATAAA ATGGGGATCG |
| | | CCTCCCAACA GCAGATTTCG ACATTACCTG AGAGTCTTGA |
| | | TTTTAGGCTT GTTTTTTGTA AACCCATGTG TTTGTAGAGA |
| | | TTTTAGGCGT CTTCGGATAT CTTCTCACCT ATGTTCCCTG |
| | | GCTAAGAAGT CAGAGGTAGC CAATGTTTCC TTAAATTCAT |
| | | TTTTAAACTT ACCATTGGTG CATATGTTCC AGATGGCAGA |
| | | TGCTGTCAAT AATCTCACCA TTGATGACCT TTGTGTATGT |
| | | AGTTCTTGCA TCCTATACTG GATAAGCCTG TTTTAACCTG |
| | | CTATGATGGG TGCTTCCATT GCTTCATAAT CTTCATGAAG |
| | | TTGCATGCTT TTGCAGCTTT TCACAGTTTA TTTGCATTTC |
| | | TAATGTAGTA ATAAAGTAAC CAATATAATC ATTA |
| 15 | ERG | ATCCGCTCTA AACAACCTCA TCAAAACTAC TTTCTGGTCA |
| | | GAGAGAAGCA ATAATTATTA TTAACATTTA TTAACGATCA |
| | | ATAAACTTGA TCGCATTATG GCCAGCACTA TTAAGGAAGC |
| | | CTTATCAGTT GTGAGTGAGG ACCAGTCGTT GTTTGAGTGT |
| | | GCCTACGGAA CGCCACACCT GGCTAAGACA GAGATGACCG |
| | | CGTCCTCCTC CAGCGACTAT GGACAGACTT CCAAGATGAG |
| | | CCCACGCGTC CCTCAGCAGG ATTGGCTGTC TCAACCCCCA |
| | | GCCAGGGTCA CCATCAAAAT GGAATGTAAC CCTAGCCAGG |
| | | TGAATGGCTC AAGGAACTCT CCTGATGAAT GCAGTGTGGC |
| | | CAAAGGCGGG AAGATGGTGG GCAGCCCAGA CACCGTTGGG |
| | | ATGAACTACG GCAGCTACAT GGAGGAGAAG CACATGCCAC |
| | | CCCCAAACAT GACCACGAAC GAGCGCAGAG TTATCGTGCC |
| | | AGCAGATCCT ACGCTATGGA GTACAGACCA TGTGCGGCAG |
| | | TGGCTGGAGT GGGCGGTGAA AGAATATGGC CTTCCAGACG |
| | | TCAACATCTT GTTATTCCAG AACATCGATG GGAAGGAACT |
| | | GTGCAAGATG ACCAAGGACG ACTTCCAGAG GCTCACCCCC |
| | | AGCTACAACG CCGACATCCT TCTCTCACAT CTCCACTACC |
| | | TCAGAGAGAC TCCTCTTCCA CATTTGACTT CAGATGATGT |
| | | TGATAAAGCC TTACAAAACT CTCCACGGTT AATGCATGCT |
| | | AGAAACACAG GGGGTGCAGC TTTTATTTTC CCAAATACTT |
| | | CAGTATATCC TGAAGCTACG CAAAGAATTA CAACTAGGCC |
| | | AGATTTACCA TATGAGCCCC CCAGGAGATC AGCCTGGACC |
| | | GGTCACGGCC ACCCCACGCC CCAGTCGAAA GCTGCTCAAC |
| | | CATCTCCTTC CACAGTGCCC AAAACTGAAG ACCAGCGTCC |
| | | TCAGTTAGAT CCTTATCAGA TTCTTGGACC AACAAGTAGC |
| | | CGCCTTGCAA ATCCAGGCAG TGGCCAGATC CAGCTTTGGC |
| | | AGTTCCTCCT GGAGCTCCTG TCGGACAGCT CCAACTCCAG |
| | | CTGCATCACC TGGGAAGGCA CCAACGGGGA GTTCAAGATG |
| | | ACGGATCCCG ACGAGGTGGC CCGGCGCTGG GGAGAGCGGA |
| | | AGAGCAAACC CAACATGAAC TACGATAAGC TCAGCCGCGC |
| | | CCTCCGTTAC TACTATGACA AGAACATCAT GACCAAGGTC |
| | | CATGGGAAGC GCTACGCCTA CAAGTTCGAC TTCCACGGGA |
| | | TCGCCCAGGC CCTCCAGCCC CACCCCCCGG AGTCATCTCT |
| | | GTACAAGTAC CCCTCAGACC TCCCGTACAT GGGCTCCTAT |
| | | CACGCCCACC CACAGAAGAT GAACTTTGTG GCGCCCCACC |
| | | CTCCAGCCCT CCCCGTGACA TCTTCCAGTT TTTTTGCTGC |
| | | CCCAAACCCA TACTGGAATT CACCAACTGG GGGTATATAC |
| | | CCCAACACTA GGCTCCCCAC CAGCCATATG CCTTCTCATC |
| | | TGGGCACTTA CTACTAAAGA CCTGGCGGAG GCTTTTCCCA |
| | | TCAGCGTGCA TTCACCAGCC CATCGCCACA AACTCTATCG |
| | | GAGAACATGA ATCAAAAGTG CCTCAAGAGG AATGAAAAAA |
| | | GCTTTACTGG GGCTGGGGAA GGAAGCCGGG GAAGAGATCC |
| | | AAAGACTCTT GGGAGGGAGT TACTGAAGTC TTACTACAGA |
| | | AATGAGGAGG ATGCTAAAAA TGTCACGAAT ATGGACATAT |
| | | CATCTGTGGA CTGACCTTGT AAAAGACAGT GTATGTAGAA |
| | | GCATGAAGTC TTAAGGACAA AGTGCCAAAG AAAGTGGTCT |
| | | TAAGAAATGT ATAAACTTTA GAGTAGAGTT TGGAATCCCA |
| | | CTAATGCAAA CTGGGATGAA ACTAAAGCAA TAGAAACAAC |
| | | ACAGTTTTGA CCTAACATAC CGTTTATAAT GCCATTTTAA |
| | | GGAAAACTAC CTGTATTTAA AAATAGAAAC ATATCAAAAA |
| | | CAAGAGAAAA GACACGAGAG AGACTGTGGC CCATCAACAG |
| | | ACGTTGATAT GCAACTGCAT GGCATGTGCT GTTTTGGTTG |
| | | AAATCAAATA CATTCCGTTT GATGGACAGC TGTCAGCTTT |
| | | CTCAAACTGT GAAGATGACC CAAAGTTTCC AACTCCTTTA |
| | | CAGTATTACC GGGACTATGA ACTAAAAGGT GGGACTGAGG |
| | | ATGTGTATAG AGTGAGCGTG TGATTGTAGA CAGAGGGGTG |
| | | AAGAAGGAGG AGGAAGAGGC AGAGAAGGAG GAGACCAGGG |
| | | CTGGGAAAGA AACTTCTCAA GCAATGAAGA CTGGACTCAG |
| | | GACATTTGGG GACTGTGTAC AATGAGTTAT GGAGACTCGA |
| | | GGGTTCATGC AGTCAGTGTT ATACCAAACC CAGTGTTAGG |
| | | AGAAAGGACA CAGCGTAATG GAGAAAGGGG AAGTAGTAGA |
| | | ATTCAGAAAC AAAAATGCGC ATCTCTTTCT TTGTTTGTCA |
| | | AATGAAAATT TTAACTGGAA TTGTCTGATA TTTAAGAGAA |
| | | ACATTCAGGA CCTCATCATT ATGTGGGGGC TTTGTTCTCC |
| | | ACAGGGTCAG GTAAGAGATG GCCTTCTTGG CTGCCACAAT |
| | | CAGAAATCAC GCAGGCATTT TGGGTAGGCG GCCTCCAGTT |
| | | TTCCTTTGAG TCGCGAACGC TGTGCGTTTG TCAGAATGAA |
| | | GTATACAAGT CAATGTTTTT CCCCCTTTTT ATATAATAAT |
| | | TATATAACTT ATGCATTTAT ACACTACGAG TTGATCTCGG |
| | | CCAGCCAAAG ACACACGACA AAAGAGACAA TCGATATAAT |
| | | GTGGCCTTGA ATTTTAACTC TGTATGCTTA ATGTTTACAA |
| | | TATGAAGTTA TTAGTTCTTA GAATGCAGAA TGTATGTAAT |
| | | AAAATAAGCT TGGCCTAGCA TGGCAAATCA GATTTATACA |
| | | GGAGTCTGCA TTTGCACTTT TTTTAGTGAC TAAAGTTGCT |
| | | TAATGAAAAC ATGTGCTGAA TGTTGTGGAT TTTGTGTTAT |
| | | AATTTACTTT GTCCAGGAAC TTGTGCAAGG GAGAGCCAAG |
| | | GAAATAGGAT GTTTGGCACC CAAATGGCGT CAGCCTCTCC |
| | | AGGTCCTTCT TGCCTCCCCT CCTGTCTTTT ATTTCTAGCC |
| | | CCTTTTGGAA CAGAAGGACC CCGGGTTTCA CATTGGAGCC |
| | | TCCATATTTA TGCCTGGAAT GGAAAGAGGC CTATGAAGCT |
| | | GGGGTTGTCA TTGAGAAATT CTAGTTCAGC ACCTGGTCAC |
| | | AAATCACCCT TAATTCCTGC TATGATTAAA ATACATTTGT |
| | | TGAACAGTGA ACAAGCTACC ACTCGTAAGG CAAACTGTAT |
| | | TATTACTGGC AAATAAAGCG TCATGGATAG CTGCAATTTC |
| | | TCACTTTACA GAAACAAGGG ATAACGTCTA GATTTGCTGC |
| | | GGGGTTTCTC TTTCAGGAGC TCTCACTAGG TAGACAGCTT |
| | | TAGTCCTGCT ACATCAGAGT TACCTGGGCA CTGTGGCTTG |
| | | GGATTCACTA GCCCTGAGCC TGATGTTGCT GGCTATCCCT |
| | | TGAAGACAAT GTTTATTTCC ATAATCTAGA GTCAGTTTCC |
| | | CTGGGCATCT TTTCTTTGAA TCACAAATGC TGCCAACCTT |
| | | GGTCCAGGTG AAGGCAACTC AAAAGGTGAA AATACAAGGT |
| | | GACCGTGCGA AGGCGCTAGC CGAAACATCT TAGCTGAATA |
| | | GGTTTCTGAA CTGGCCCTTT TCATAGCTGT TTCAGGGCCT |
| | | GTTTTTTTCA CGTTGCAGTC CTTTTGCTAT GATTATGTGA |
| | | AGTTGCCAAA CCTCTGTGCT GTGGATGTTT TGGCAGTGGG |
| | | CTTTGAAGTC GGCAGGACAC GATTACCAAT GCTCCTGACA |
| | | CCCCGTGTCA TTTGGATTAG ACGGAGCCCA ACCATCCATC |
| | | ATTTTGCAGC AGCCTGGGAA GGCCCACAAA GTGCCCGTAT |
| | | CTCCTTAGGG AAAATAAATA AATACAATCA TGAAAGCTGG |
| | | CAGTTAGGCT GACCCAAACT GTGCTAATGG AAAAGATCAG |
| | | TCATTTTTAT TTTGGAATGC AAAGTCAAGA CACACCTACA |
| | | TTCTTCATAG AAATACACAT TTACTTGGAT AATCACTCAG |
| | | TTCTCTCTTC AAGACTGTCT CATGAGCAAG ATCATAAAAA |
| | | CAAGACATGA TTATCATATT CAATTTTAAC AGATGTTTTC |
| | | CATTAGATCC CTCAACCCTC CACCCCCAGT CCAGGTTATT |
| | | AGCAAGTCTT ATGAGCAACT GGGATAATTT TGGATAACAT |
| | | GATAATACTG AGTTCCTTCA AATACATAAT TCTTAAATTG |
| | | TTTCAAAATG GCATTAACTC TCTGTTACTG TTGTAATCTA |
| | | ATTCCAAAGC CCCCTCCAGG TCATATTCAT AATTGCATGA |
| | | ACCTTTTCTC TCTGTTTGTC CCTGTCTCTT GGCTTGCCCT |
| | | GATGTATACT CAGACTCCTG TACAATCTTA CTCCTGCTGG |
| | | CAAGAGATTT GTCTTCTTTT CTTGTCTTCA ATTGGCTTTC |
| | | GGGCCTTGTA TGTGGTAAAA TCACCAAATC ACAGTCAAGA |
| | | CTGTGTTTTT GTTCCTAGTT TGATGCCCTT ATGTCCCGGA |
| | | GGGGTTCACA AAGTGCTTTG TCAGGACTGC TGCAGTTAGA |
| | | AGGCTCACTG CTTCTCCTAA GCCTTCTGCA CAGATGTGGC |
| | | ACCTGCAACC CAGGAGCAGG AGCCGGAGGA GCTGCCCTCT |
| | | GACAGCAGGT GCAGCAGAGA TGGCTACAGC TCAGGAGCTG |
| | | GGAAGGTGAT GGGGCACAGG GAAAGCACAG ATGTTCTGCA |
| | | GCGCCCCAAA GTGACCCATT GCCTGGAGAA AGAGAAGAAA |
| | | ATATTTTTTA AAAAGCTAGT TTATTTAGCT TCTCATTAAT |
| | | TCATTCAAAT AAAGTCGTGA GGTGACTAAT TAGAGAATAA |
| | | AAATTACTTT GGACTACTCA AAAA |
| 16 | KLK4 | AGGCAGCAGG CTGGAGCTCA GCCCAGCAGT GGAATCCAGG |
| | | AGCCCAGAGG TGGCCGGGTG CTGACGTGAT GGCCACAGCA |
| | | GGAAATCCCT GGGGCTGGTT CCTGGGGTAC CTCATCCTTG |
| | | GTGTCGCAGG ATCGCTCGTC TCTGGTAGCT GCAGCCAAAT |
| | | CATAAACGGC GAGGACTGCA GCCCGCACTC GCAGCCCTGG |
| | | CAGGCGGCAC TGGTCATGGA AAACGAATTG TTCTGCTCGG |
| | | GCGTCCTGGT GCATCCGCAG TGGGTGCTGT CAGCCGCACA |
| | | CTGTTTCCAG AACTCCTACA CCATCGGGCT GGGCCTGCAC |
| | | AGTCTTGAGG CCGACCAAGA GCCAGGGAGC CAGATGGTGG |
| | | AGGCCAGCCT CTCCGTACGG CACCCAGAGT ACAACAGACC |
| | | CTTGCTCGCT AACGACCTCA TGCTCATCAA GTTGGACGAA |
| | | TCCGTGTCCG AGTCTGACAC CATCCGGAGC ATCAGCATTG |
| | | CTTCGCAGTG CCCTACCGCG GGGAACTCTT GCCTCGTTTC |
| | | TGGCTGGGGT CTGCTGGCGA ACGGCAGAAT GCCTACCGTG |
| | | CTGCAGTGCG TGAACGTGTC GGTGGTGTCT GAGGAGGTCT |
| | | GCAGTAAGCT CTATGACCCG CTGTACCACC CCAGCATGTT |
| | | CTGCGCCGGC GGAGGGCAAG ACCAGAAGGA CTCCTGCAAC |
| | | GGTGACTCTG GGGGGCCCCT GATCTGCAAC GGGTACTTGC |
| | | AGGGCCTTGT GTCTTTCGGA AAAGCCCCGT GTGGCCAAGT |
| | | TGGCGTGCCA GGTGTCTACA CCAACCTCTG CAAATTCACT |
| | | GAGTGGATAG AGAAAACCGT CCAGGCCAGT TAACTCTGGG |
| | | GACTGGGAAC CCATGAAATT GACCCCCAAA TACATCCTGC |
| | | GGAAGGAATT CAGGAATATC TGTTCCCAGC CCCTCCTCCC |
| | | TCAGGCCCAG GAGTCCAGGC CCCCAGCCCC TCCTCCCTCA |
| | | AACCAAGGGT ACAGATCCCC AGCCCCTCCT CCCTCAGACC |
| | | CAGGAGTCCA GACCCCCCAG CCCCTCCTCC CTCAGACCCA |
| | | GGAGTCCAGC CCCTCCTCCC TCAGACCCAG GAGTCCAGAC |
| | | CCCCCAGCCC CTCCTCCCTC AGACCCAGGA GTCCAGCCCC |
| | | TCCTCCCTCA GACCCAGGAG TCCAGACCCC CCAGCCCCTC |
| | | CTCCCTCAGA CCCAGGGGTC CAGGCCCCCA ACCCCTCCTC |
| | | CCTCAGACTC AGAGGTCCAG GCCCCCAACC CCTCCTTCCC |
| | | CAGACCCAGA GGTCCAGGTC CCAGCCCCTC CTCCCTCAGA |
| | | CCCAGCGGTC CAATGCCACC TAGACTCTCC CTGTACACAG |
| | | TGCCCCCTTG TGGCACGTTG ACCCAACCTT ACCAGTTGGT |
| | | TTTTCATTTT TTGTCCCTTT CCCCTAGATC CAGAAATAAA |
| | | GTCTAAGAGA AGCGCA |
| 17 | HOXC6 | ATAACCATCT AGTTCCGAGT ACAAACTGGA GACAGAAATA |
| | | AATATTAAAG AAATCATAGA CCGACCAGGT AAAGGCAAAG |
| | | GGATGAATTC CTACTTCACT AACCCTTCCT TATCCTGCCA |
| | | CCTCGCCGGG GGCCAGGACG TCCTCCCCAA CGTCGCCCTC |
| | | AATTCCACCG CCTATGATCC AGTGAGGCAT TTCTCGACCT |
| | | ATGGAGCGGC CGTTGCCCAG AACCGGATCT ACTCGACTCC |
| | | CTTTTATTCG CCACAGGAGA ATGTCGTGTT CAGTTCCAGC |
| | | CGGGGGCCGT ATGACTATGG ATCTAATTCC TTTTACCAGG |
| | | AGAAAGACAT GCTCTCAAAC TGCAGACAAA ACACCTTAGG |
| | | ACATAACACA CAGACCTCAA TCGCTCAGGA TTTTAGTTCT |
| | | GAGCAGGGCA GGACTGCGCC CCAGGACCAG AAAGCCAGTA |
| | | TCCAGATTTA CCCCTGGATG CAGCGAATGA ATTCGCACAG |
| | | TGGGGTCGGC TACGGAGCGG ACCGGAGGCG CGGCCGCCAG |
| | | ATCTACTCGC GGTACCAGAC CCTGGAACTG GAGAAGGAAT |
| | | TTCACTTCAA TCGCTACCTA ACGCGGCGCC GGCGCATCGA |
| | | GATCGCCAAC GCGCTTTGCC TGACCGAGCG ACAGATCAAA |
| | | ATCTGGTTCC AGAACCGCCG GATGAAGTGG AAAAAAGAAT |
| | | CTAATCTCAC ATCCACTCTC TCGGGGGGCG GCGGAGGGGC |
| | | CACCGCCGAC AGCCTGGGCG GAAAAGAGGA AAAGCGGGAA |
| | | GAGACAGAAG AGGAGAAGCA GAAAGAGTGA CCAGGACTGT |
| | | CCCTGCCACC CCTCTCTCCC TTTCTCCCTC GCTCCCCACC |
| | | AACTCTCCCC TAATCACACA CTCTGTATTT ATCACTGGCA |
| | | CAATTGATGT GTTTTGATTC CCTAAAACAA AATTAGGGAG |
| | | TCAAACGTGG ACCTGAAAGT CAGCTCTGGA CCCCCTCCCT |
| | | CACCGCACAA CTCTCTTTCA CCACGCGCCT CCTCCTCCTC |
| | | GCTCCCTTGC TAGCTCGTTC TCGGCTTGTC TACAGGCCCT |
| | | TTTCCCCGTC CAGGCCTTGG GGGCTCGGAC CCTGAACTCA |
| | | GACTCTACAG ATTGCCCTCC AAGTGAGGAC TTGGCTCCCC |
| | | CACTCCTTCG ACGCCCCCAC CCCCGCCCCC CGTGCAGAGA |
| | | GCCGGCTCCT GGGCCTGCTG GGGCCTCTGC TCCAGGGCCT |
| | | CAGGGCCCGG CCTGGCAGCC GGGGAGGGCC GGAGGCCCAA |
| | | GGAGGGCGCG CCTTGGCCCC ACACCAACCC CCAGGGCCTC |
| | | CCCGCAGTCC CTGCCTAGCC CCTCTGCCCC AGCAAATGCC |
| | | CAGCCCAGGC AAATTGTATT TAAAGAATCC TGGGGGTCAT |
| | | TATGGCATTT TACAAACTGT GACCGTTTCT GTGTGAAGAT |
| | | TTTTAGCTGT ATTTGTGGTC TCTGTATTTA TATTTATGTT |
| | | TAGCACCGTC AGTGTTCCTA TCCAATTTCA AAAAAGGAAA |
| | | AAAAAGAGGG AAAATTACAA AAAGAGAGAA AAAAAGTGAA |
| | | TGACGTTTGT TTAGCCAGTA GGAGAAAATA AATAAATAAA |
| | | TAAATCCCTT CGTGTTACCC TCCTGTATAA ATCCAACCTC |
| | | TGGGTCCGTT CTCGAATATT TAATAAAACT GATATTATTT |
| | | TTAAAACTTT A |

In some embodiments, the methods described herein are useful for detecting an amount of expression of 1, 2, 3, 4, 5, 6, 7, 8, 9, 10, 11, 12, 13, 14, 15, 16 or 17 genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

In some embodiments, the methods described herein comprise detecting an amount of expression of at least three genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least three genes are TMPRSS2-ERG, PCA3 and PCAT14.

In some embodiments, the methods described herein comprise detecting an amount of expression of at least four genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least four genes are TMPRSS2-ERG, PCA3, PCAT14, and OR51E2. In some embodiments, the at least four genes are TMPRSS2-ERG, PCA3, PCAT14, and TRGV9. In some embodiments, the at least four genes are TMPRSS2-ERG, PCA3, PCAT14, and ERG. In some embodiments, the at least four genes are TMPRSS2-ERG, PCA3, PCAT14, and TFF3. In some embodiments, the at least four genes are TMPRSS2-ERG, PCA3, PCAT14, and SCHLAP1. In some embodiments, the at least four genes are TMPRSS2-ERG, PCA3, PCAT14, and HOXC6. In some embodiments, the at least four genes are TMPRSS2-ERG, PCA3, PCAT14, and SPON2. In some embodiments, the at least four genes are TMPRSS2-ERG, PCA3, PCAT14, and TMSB15A. In some embodiments, the at least four genes are TMPRSS2-ERG, PCA3, PCAT14, and APOC1. In some embodiments, the at least four genes are TMPRSS2-ERG, PCA3, PCAT14, and B3GNT6. In some embodiments, the at least four genes are TMPRSS2-ERG, PCA3, PCAT14, and KLK4. In some embodiments, the at least four genes are TMPRSS2-ERG, PCA3, PCAT14, and CAMKK2. In some embodiments, the at least four genes are TMPRSS2-ERG, PCA3, PCAT14, and NKAIN1. In some embodiments, the at least four genes are TMPRSS2-ERG, PCA3, PCAT14, and PCGEM1.

In some embodiments, the methods described herein comprise detecting an amount of expression of at least five genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least five genes are TMPRSS2-ERG, PCA3, PCAT14, OR51E2 and TRGV9. In some embodiments, the at least five genes are TMPRSS2-ERG, PCA3, PCAT14, ERG and OR51E2. In some embodiments, the at least five genes are TMPRSS2-ERG, PCA3, PCAT14, TFF3 and TRGV9. In some embodiments, the at least five genes are TMPRSS2-ERG, PCA3, PCAT14, OR51E2, and TFF3. In some embodiments, the at least five genes are TMPRSS2-ERG, PCA3, PCAT14, SCHLAP1, and TRGV9. In some embodiments, the at least five genes are TMPRSS2-ERG, PCA3, PCAT14, OR51E2 and SCHLAP1. In some embodiments, the at least five genes are TMPRSS2-ERG, PCA3, PCAT14, HOXC6 and TRGV9. In some embodiments, the at least five genes are TMPRSS2-ERG, PCA3, PCAT14, OR51E2 and HOXC6. In some embodiments, the at least five genes are TMPRSS2-ERG, PCA3, PCAT14, SPON2, and TRGV9. In some embodiments, the at least five genes are TMPRSS2-ERG, PCA3, PCAT14, ERG and SPON2. In some embodiments, the at least five genes are TMPRSS2-ERG, PCA3, PCAT14, TMPSB15A and TRGV9. In some embodiments, the at least five genes are TMPRSS2-ERG, PCA3, PCAT14, ERG and TMSB15A. In some embodiments, the at least five genes are TMPRSS2-ERG, PCA3, PCAT14, APOC1, and TRGV9. In some embodiments, the at least five genes are TMPRSS2-ERG, PCA3, PCAT14, ERG and APOC1. In some embodiments, the at least five genes are TMPRSS2-ERG, PCA3, PCAT14, B3GNT6 and TRGV9. In some embodiments, the at least five genes are TMPRSS2-ERG, PCA3, PCAT14, ERG and B3GNT6. In some embodiments, the at least five genes are TMPRSS2-ERG, PCA3, PCAT14, KLK4 and TRGV9. In some embodiments, the at least five genes are TMPRSS2-ERG, PCA3, PCAT14, ERG and KLK4. In some embodiments, the at least five genes are TMPRSS2-ERG, PCA3, PCAT14, CAMKK2 and TRGV9. In some embodiments, the at least five genes are TMPRSS2-ERG, PCA3, PCAT14, ERG and CAMKK2. In some embodiments, the at least five genes are TMPRSS2-ERG, PCA3, PCAT14, NKAIN1, and TRGV9. In some embodiments, the at least five genes are TMPRSS2-ERG, PCA3, PCAT14, ERG and NKAIN1. In some embodiments, the at least five genes are TMPRSS2-ERG, PCA3, PCAT14, PCGEM1 and TRGV9. In some embodiments, the at least five genes are TMPRSS2-ERG, PCA3, PCAT14, ERG and PCGEM1.

In some embodiments, the methods described herein comprise detecting an amount of expression of at least six genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least six genes are TMPRSS2-ERG, PCA3, PCAT14, ERG, TRGV9 and OR51E2. In some embodiments, the at least six genes are TMPRSS2-ERG, PCA3, PCAT14, ERG, NKAIN1, and PCGEM1. In some embodiments, the at least six genes are TMPRSS2-ERG, PCA3, PCAT14, TRGV9 and PCGEM1. In some embodiments, the at least six genes are TMPRSS2-ERG, PCA3, PCAT14, ERG, CAMKK2 and PCGEM1. In some embodiments, the at least six genes are TMPRSS2-ERG, PCA3, PCAT14, ERG, TRGV9 and NKAIN1. In some embodiments, the at least six genes are TMPRSS2-ERG, PCA3, PCAT14, ERG, OR51E2 and TCGEM1. In some embodiments, the at least six genes are TMPRSS2-ERG, PCA3, PCAT14, ERG, TRGV9 and TFF3. In some embodiments, the at least six genes are TMPRSS2-ERG, PCA3, PCAT14, ERG, OR51E2 and SCHLAP1. In some embodiments, the at least six genes are TMPRSS2-ERG, PCA3, PCAT14, ERG, TFF3 and HOXC6. In some embodiments, the at least six genes are TMPRSS2-ERG, PCA3, PCAT14, ERG, SCHLAP1 and SPON2. In some embodiments, the at least six genes are TMPRSS2-ERG, PCA3, PCAT14, ERG, HOXC6 and TMSB15A. In some embodiments, the at least six genes are TMPRSS2-ERG, PCA3, PCAT14, ERG, SPON2 and APOC. In some embodiments, the at least six genes are TMPRSS2-ERG, PCA3, PCAT14, ERG, TMSB15A and APOC1. In some embodiments, the at least six genes are TMPRSS2-ERG, PCA3, PCAT14, ERG, TMSB15A and B3GNT6. In some embodiments, the at least six genes awe TMPRSS2-ERG, PCA3, PCAT14, ERG, APOC1 and KLK4. In some embodiments, the at least six genes are TMPRSS2-ERG, PCA3, PCAT14, ERG, B3GNT6 and CAMKK2. In some embodiments, the at least six genes are TMPRSS2-ERG, PCA3, PCAT14, ERG, KLK4 and NKAIN1. In some embodiments, the at least six genes are TMPRSS2-ERG, PCA3, PCAT14, ERG, CAMKK2 and PCGEM1. In some embodiments, the at least six genes are TMPRSS2-ERG, PCA3, PCAT14, ERG, KLK4 and PCGEM1. In some embodiments, the at least six genes are TMPRSS2-ERG, PCA3, PCAT14, ERG, TRGV9 and CAMKK12. In some embodiments, the at least six genes are TMPRSS2-ERG, PCA3, PCAT14, ERG, OR51E2 and NKAIN1. In some embodiments, the at least six genes are TMPRSS2-ERG, PCA3, PCAT14, ERG, TFF3 and PCGEM1. In some embodiments, the at least six genes are TMPRSS2-ERG, PCA3, PCAT14, ERG, TRGV9 and SCHLAP1. In some embodiments, the at least six genes are TMPRSS2-ERG, PCA3, PCAT14, ERG, OR51E2 and HOXC6. In some embodiments, the at least six genes are TMPRSS2-ERG, PCA3, PCAT14, ERG, TFF3 and SPON2. In some embodiments, the at least six genes are TMPRSS2-ERG, PCA3, PCAT14, ERG, SCHLAP1 and TMSB15A. In some embodiments, the at least six genes are TMPRSS2-ERG, PCA3, PCAT14, ERG, HOXC6 and APOC1. In some embodiments, the at least six genes awe TMPRSS2-ERG, PCA3, PCAT14, ERG, SPON2 and B2GNT6. In some embodiments, the at least six genes are TMPRSS2-ERG, PCA3, PCAT14, ERG, TMSB15A and KLK4. In some embodiments, the at least six genes are TMPRSS2-ERG, PCA3, PCAT14, ERG, APOC1 and CAMKK2. In some embodiments, the at least six genes awe TMPRSS2-ERG, PCA3, PCAT14, ERG, B3GNT6 and NKAIN1. In some embodiments, the at least six genes are TMPRSS2-ERG, PCA3, PCAT14, ERG, B3GNT6 and PCGEM1. In some embodiments, the at least six genes are TMPRSS2-ERG, PCA3, PCAT14, ERG, TRGV9 and KLK4. In some embodiments, the at least six genes awe TMPRSS2-ERG, PCA3, PCAT14, ERG, OR51E2 and CAMKK2. In some embodiments, the at least six genes are TMPRSS2-ERG, PCA3, PCAT14, ERG, TFF3 and NKAIN1. In some embodiments, the at least six genes awe TMPRSS2-ERG, PCA3, PCAT14, ERG, SCHLAP1 and PCGEM1. In some embodiments, the at least six genes are TMPRSS2-ERG, PCA3, PCAT14, ERG, TRGV9 and HOXC6. In some embodiments, the at least six genes awe TMPRSS2-ERG, PCA3, PCAT14, ERG, OR51E2 and SPON2. In some embodiments, the at least six genes are TMPRSS2-ERG, PCA3, PCAT14, ERG, TFF3 and TMSB15A. In some embodiments, the at least six genes awe TMPRSS2-ERG, PCA3, PCAT14, ERG, SCHLAP1 and APOC1. In some embodiments, the at least six genes are TMPRSS2-ERG, PCA3, PCAT14, ERG, HOXC6 and B3GNT6. In some embodiments, the at least six genes are TMPRSS2-ERG, PCA3, PCAT14, ERG, SPON2 and KLK4. In some embodiments, the at least six genes are TMPRSS2-ERG, PCA3, PCAT14, ERG, TMSB15A and CAMKK2. In some embodiments, the at least six genes are TMPRSS2-ERG, PCA3, PCAT14, ERG, APOC1 and NKAIN1. In some embodiments, the at least six genes are TMPRSS2-ERG, PCA3, PCAT14, ERG, APOC1 and PCGEM1. In some embodiments, the at least six genes awe TMPRSS2-ERG, PCA3, PCAT14, ERG, TRGV9 and B3GNT6. In some embodiments, the at least six genes are TMPRSS2-ERG, PCA3, PCAT14, ERG, OR51E2 and KLK4. In some embodiments, the at least six genes are TMPRSS2-ERG, PCA3, PCAT14, ERG, TFF3 and CAMKK2. In some embodiments, the at least six genes are TMPRSS2-ERG, PCA3, PCAT14, ERG, SCHLAP1 and NKAIN1. In some embodiments, the at least six genes are TMPRSS2-ERG, PCA3, PCAT14, ERG, HOXC6 and PCGEM1. In some embodiments, the at least six genes are TMPRSS2-ERG, PCA3, PCAT14, ERG, HOXC6 and SPON2. In some embodiments, the at least six genes are TMPRSS2-ERG, PCA3, PCAT14, ERG, OR51E2 and TMSB15A. In some embodiments, the at least six genes are TMPRSS2-ERG, PCA3, PCAT14, ERG, TMSB15A and PCGEM1. In some embodiments, the at least six genes awe TMPRSS2-ERG, PCA3, PCAT14, ERG, TRGV9 and APOC1. In some embodiments, the at least six genes are TMPRSS2-ERG, PCA3, PCAT14, ERG, OR51E2 and B3GNT6. In some embodiments, the at least six genes are TMPRSS2-ERG, PCA3, PCAT14, ERG, TFF3 and KLK4. In some embodiments, the at least six genes are TMPRSS2-ERG, PCA3, PCAT14, ERG, SCHLAP1 and CAMKK2. In some embodiments, the at least six genes are TMPRSS2-ERG, PCA3, PCAT14, ERG, HOXC6 and NKAIN1. In some embodiments, the at least six genes are TMPRSS2-ERG, PCA3, PCAT14, ERG, SPON2 and PCGEM1.

In some embodiments, the methods described herein comprise detecting an amount of expression of at least seven genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least seven genes are TMPRSS2-ERG, PCA3, PCAT14, ERG, TRGV9, OR51E2 and TFF3. In some embodiments, the at least seven genes are TMPRSS2-ERG, PCA3, PCAT14, ERG, TRGV9, NKAIN1 and PCGEM1. In some embodiments, the at least seven genes are TMPRSS2-ERG, PCA3, PCAT14, ERG, TRGV9, OR51E2 and PCGEM1. In some embodiments, the at least seven genes are TMPRSS2-ERG, PCA3, PCAT14, ERG, TRGV9, CAMKK2, and PCGEM1. In some embodiments, the at least seven genes are TMPRSS2-ERG, PCA3, PCAT14, ERG, TRGV9, OR51E2 and NKAIN1. In some embodiments, the at least seven genes are TMPRSS2-ERG, PCA3, PCAT14, ERG, TRGV9, KLK4 and PCGEM1. In some embodiments, the at least seven genes are TMPRSS2-ERG, PCA3, PCAT14, ERG, TRGV9, OR51E2 and CAMKK2. In some embodiments, the at least seven genes are TMPRSS2-ERG, PCA3, PCAT14, ERG, TRGV9, SCHLAP1 and HOXC6. In some embodiments, the at least seven genes are TMPRSS2-ERG, PCA3, PCAT14, ERG, TRGV9, B3GNT6 and PCGEM1. In some embodiments, the at least seven genes are TMPRSS2-ERG, PCA3, PCAT14, ERG, TRGV9, OR51E2 and KLK4. In some embodiments, the at least seven genes are TMPRSS2-ERG, PCA3, PCAT14, ERG, TRGV9, HOXC6 and SPON2. In some embodiments, the at least seven genes are TMPRSS2-ERG, PCA3, PCAT14, ERG, TRGV9, APOC1 and PCGEM1. In some embodiments, the at least seven genes are TMPRSS2-ERG, PCA3, PCAT14, ERG, TRGV9, OR51E2 and B3GNT6. In some embodiments, the at least seven genes are TMPRSS2-ERG, PCA3, PCAT14, ERG, TRGV9, TMSB15A and PCGEM1. In some embodiments, the at least seven genes are TMPRSS2-ERG, PCA3, PCAT14, ERG, TRGV9, OR51E2 and APOC1. In some embodiments, the at least seven genes are TMPRSS2-ERG, PCA3, PCAT14, ERG, KLK4, NKAIN1 and PCGEM1. In some embodiments, the at least seven genes are TMPRSS2-ERG, PCA3, PCAT14, ERG, B3GNT6, NKAIN1, and PCGEM1. In some embodiments, the at least seven genes are TMPRSS2-ERG, PCA3, PCAT14, ERG, OR51E2, TFF3 and NKAIN1. In some embodiments, the at least seven genes are TMPRSS2-ERG, PCA3, PCAT14, ERG, OR51E2, HOXC6 and SPON2. In some embodiments, the at least seven genes are TMPRSS2-ERG, PCA3, PCAT14, ERG, OR51E2, TFF3 and CAMKK2. In some embodiments, the at least seven genes are TMPRSS2-ERG, PCA3, PCAT14, ERG, OR51E2, SPON2 and TMPSB15A. In some embodiments, the at least seven genes are TMPRSS2-ERG, PCA3, PCAT14, ERG, OR51E2, TFF3 and KLK4. In some embodiments, the at least seven genes are TMPRSS2-ERG, PCA3, PCAT14, ERG, TFF3, SCHLAP1 and PCGEM1. In some embodiments, the at least seven genes are TMPRSS2-ERG, PCA3, PCAT14, ERG, TFF3, SPON2 and TMSB15A. In some embodiments, the at least seven genes are TMPRSS2-ERG, PCA3, PCAT14, ERG, TFF3, SCHLAP1 and NKAIN1. In some embodiments, the at least seven genes are TMPRSS2-ERG, PCA3, PCAT14, ERG, TFF3, TMSB15A and APOC1. In some embodiments, the at least seven genes are TMPRSS2-ERG, PCA3, PCAT14, ERG, TFF3, SCHLAP1 and CAMKK2. In some embodiments, the at least seven genes are TMPRSS2-ERG, PCA3, PCAT14, ERG, SCHLAP1, TMSB15A and APOC1. In some embodiments, the at least seven genes are TMPRSS2-ERG, PCA3, PCAT14, ERG, SCHLAP1, HOXC6 and PCGEM1. In some embodiments, the at least seven genes awe TMPRSS2-ERG, PCA3, PCAT14, ERG, SCHLAP1, APOC1 and B3GNT6. In some embodiments, the at least seven genes are TMPRSS2-ERG, PCA3, PCAT14, ERG, SCHLAP1, HOXC6 and NKAIN1. In some embodiments, the at least seven genes are TMPRSS2-ERG, PCA3, PCAT14, ERG, HOXC6, APOC1 and B3GNT6. In some embodiments, the at least seven genes are TMPRSS2-ERG, PCA3, PCAT14, ERG, HOXC6, B3GNT6 and KLK4. In some embodiments, the at least seven genes awe TMPRSS2-ERG, PCA3, PCAT14, ERG, HOXC6, SPON2 and PCGEM1. In some embodiments, the at least seven genes are TMPRSS2-ERG, PCA3, PCAT14, ERG, SPON2, B3GNT6 and KLK4. In some embodiments, the at least seven genes are TMPRSS2-ERG, PCA3, PCAT14, ERG, SPON2, KLK4 and CAMKK2. In some embodiments, the at least seven genes are TMPRSS2-ERG, PCA3, PCAT14, ERG, SPON2, NKAIN1 and PCGEM1. In some embodiments, the at least seven genes are TMPRSS2-ERG, PCA3, PCAT14, ERG, TMSB15A, KLK4 and CAMKK2. In some embodiments, the at least seven genes are TMPRSS2-ERG, PCA3, PCAT14, ERG, TMSB15A, CAMKK2 and NKAIN1. In some embodiments, the at least seven genes are TMPRSS2-ERG, PCA3, PCAT14, ERG, TMSB15A, NKAIN1 and PCGEM1. In some embodiments, the at least seven genes are TMPRSS2-ERG, PCA3, PCAT14, ERG, APOC1, NKAIN1, and CAMKK2. In some embodiments, the at least seven genes are TMPRSS2-ERG, PCA3, PCAT14, ERG, APOC1, NKAIN1 and PCGEM1.

In some embodiments, the methods described herein comprise detecting an amount of expression of at least eight genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least eight genes are TMPRSS2-ERG, PCA3, PCAT14, ERG, TRGV9, OR51E2, TFF3 and SCHLAP1. In some embodiments, the at least eight genes are TMPRSS2-ERG, PCA3, PCAT14, ERG, TRGV9, OR51E2, NKAIN1, and PCGEM1. In some embodiments, the at least eight genes are TMPRSS2-ERG, PCA3, PCAT14, ERG, TRGV9, OR51E2, TFF3 and PCGEM1. In some embodiments, the at least eight genes are TMPRSS2-ERG, PCA3, PCAT14, ERG, TRGV9, OR51E2, CAMKK2 and PCGEM1. In some embodiments, the at least eight genes are TMPRSS2-ERG, PCA3, PCAT14, ERG, TRGV9, OR51E2, TFF3 and NKAIN1. In some embodiments, the at least eight genes are TMPRSS2-ERG, PCA3, PCAT14, ERG, TRGV9, OR51E2, SCHLAP1 and HOXC6. In some embodiments, the at least eight genes are TMPRSS2-ERG, PCA3, PCAT14, ERG, TRGV9, OR51E2, KLK4 and PCGEM1. In some embodiments, the at least eight genes are TMPRSS2-ERG, PCA3, PCAT14, ERG, TRGV9, OR51E2, TFF3 and CAMKK2. In some embodiments, the at least eight genes are TMPRSS2-ERG, PCA3, PCAT14, ERG, TRGV9, OR51E2, HOXC6 and SPON2. In some embodiments, the at least eight genes are TMPRSS2-ERG, PCA3, PCAT14, ERG, TRGV9, OR51E2, B3GNT6 and PCGEM1. In some embodiments, the at least eight genes are TMPRSS2-ERG, PCA3, PCAT14, ERG, TRGV9, OR51E2, TFF3 and KLK4. In some embodiments, the at least eight genes are TMPRSS2-ERG, PCA3, PCAT14, ERG, TRGV9, OR51E2, SPON2 and PCGEM1. In some embodiments, the at least eight genes are TMPRSS2-ERG, PCA3, PCAT14, ERG, TRGV9, OR51E2, APOC1 and PCGEM1. In some embodiments, the at least eight genes are TMPRSS2-ERG, PCA3, PCAT14, ERG, TRGV9, OR51E2, TFF3 and B2GNT6. In some embodiments, the at least eight genes are TMPRSS2-ERG, PCA3, PCAT14, ERG, TRGV9, CAMKK2, NKAIN1 and PCGEM1. In some embodiments, the at least eight genes are TMPRSS2-ERG, PCA3, PCAT14, ERG, TRGV9, KLK4, NKAIN1 and PCGEM1. In some embodiments, the at least eight genes are TMPRSS2-ERG, PCA3, PCAT14, ERG, TRGV9, TFF3, SCHLAP1 and PCGEM1. In some embodiments, the at least eight genes are TMPRSS2-ERG, PCA3, PCAT14, ERG, TRGV9, TFF3, HOXC6 and SPON2. In some embodiments, the at least eight genes are TMPRSS2-ERG, PCA3, PCAT14, ERG, TRGV9, TFF3, SCHLAP1 and NKAIN1. In some embodiments, the at least eight genes are TMPRSS2-ERG, PCA3, PCAT14, ERG, TRGV9, TFF3, SPON2 and TMSB15A. In some embodiments, the at least eight genes are TMPRSS2-ERG, PCA3, PCAT14, ERG, TRGV9, TFF3, SCHLAP1 and CAMKK2. In some embodiments, the at least eight genes are TMPRSS2-ERG, PCA3, PCAT14, ERG, TRGV9, TFF3, SCHLAP1 and KLK4. In some embodiments, the at least eight genes are TMPRSS2-ERG, PCA3, PCAT14, ERG, TRGV9, SCHLAP1, SPON2 and TMSB15A. In some embodiments, the at least eight genes are TMPRSS2-ERG, PCA3, PCAT14, ERG, TRGV9, SCHLAP1, HOXC6 and PCGEM1. In some embodiments, the at least eight genes are TMPRSS2-ERG, PCA3, PCAT14, ERG, TRGV9, SCHLAP1, TMSB15A and APOC1. In some embodiments, the at least eight genes are TMPRSS2-ERG, PCA3, PCAT14, ERG, TRGV9, SCHLAP1, HOXC6 and NKAIN1. In some embodiments, the at least eight genes are TMPRSS2-ERG, PCA3, PCAT14, ERG, TRGV9, SCHLAP1, HOXC6 and CAMKK2. In some embodiments, the at least eight genes are TMPRSS2-ERG, PCA3, PCAT14, ERG, TRGV9, HOXC6, TMSB15A and APOC1. In some embodiments, the at least eight genes are TMPRSS2-ERG, PCA3, PCAT14, ERG, TRGV9, HOXC6, APOC1 and B3GNT6. In some embodiments, the at least eight genes are TMPRSS2-ERG, PCA3, PCAT14, ERG, TRGV9, HOXC6, SPON2 and NKAIN1. In some embodiments, the at least eight genes are TMPRSS2-ERG, PCA3, PCAT14, ERG, TRGV9, SPON2, APOC1 and B3GNT6. In some embodiments, the at least eight genes are TMPRSS2-ERG, PCA3, PCAT14, ERG, TRGV9, SPON2, B3GNT6 and KLK4. In some embodiments, the at least eight genes are TMPRSS2-ERG, PCA3, PCAT14, ERG, TRGV9, SPON2, TMSB15A and PCGEM1. In some embodiments, the at least eight genes are TMPRSS2-ERG, PCA3, PCAT14, ERG, TRGV9, TMSB15A, B3GNT6 and KLK4. In some embodiments, the at least eight genes are TMPRSS2-ERG, PCA3, PCAT14, ERG, TRGV9, TMSB15A, KLK4 and CAMKK2. In some embodiments, the at least eight genes are TMPRSS2-ERG, PCA3, PCAT14, ERG, TRGV9, TMSB15A, NKAIN1 and PCGEM1. In some embodiments, the at least eight genes are TMPRSS2-ERG, PCA3, PCAT14, ERG, TRGV9, APOC1, KLK4 and CAMKK2. In some embodiments, the at least eight genes are TMPRSS2-ERG, PCA3, PCAT14, ERG, TRGV9, APOC1, CAMKK2 and NKAIN1. In some embodiments, the at least eight genes are TMPRSS2-ERG, PCA3, PCAT14, ERG, TRGV9, APOC1, NKAIN1 and PCGEM1. In some embodiments, the at least eight genes are TMPRSS2-ERG, PCA3, PCAT14, ERG, TRGV9, B3GNT6, CAMKK2 and NKAIN1. In some embodiments, the at least eight genes are TMPRSS2-ERG, PCA3, PCAT14, ERG, TRGV9, B3GNT6, NKAIN1 and PCGEM1.

In some embodiments, the methods described herein comprise detecting an amount of expression of at least nine genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least nine genes awe TMPRSS2-ERG, PCA3, PCAT14, ERG, TRGV9, OR51E2, TFF3, SCHLAP1 and HOXC6. In some embodiments, the at least nine genes are TMPRSS2-ERG, PCA3, PCAT14, ERG, TRGV9, OR51E2, TFF3, SCHLAP1 and PCGEM1. In some embodiments, the at least nine genes are TMPRSS2-ERG, PCA3, PCAT14, ERG, TRGV9, OR51E2, TFF3, SCHLAP1 and NKAIN1. In some embodiments, the at least nine genes are TMPRSS2-ERG, PCA3, PCAT14, ERG, TRGV9, OR51E2, TFF3, SCHLAP1 and SPON2. In some embodiments, the at least nine genes awe TMPRSS2-ERG, PCA3, PCAT14, ERG, TRGV9, OR51E2, TFF3, SCHLAP1 and CAMKK2. In some embodiments, the at least nine genes are TMPRSS2-ERG, PCA3, PCAT14, ERG, TRGV9, OR51E2, TFF3, SCHLAP1 and HOXC6. In some embodiments, the at least nine genes awe TMPRSS2-ERG, PCA3, PCAT14, ERG, TRGV9, OR51E2, TFF3, SCHLAP1 and TMSB15A. In some embodiments, the at least nine genes awe TMPRSS2-ERG, PCA3, PCAT14, ERG, TRGV9, OR51E2, TFF3, SCHLAP1 and KLK4. In some embodiments, the at least nine genes awe TMPRSS2-ERG, PCA3, PCAT14, ERG, TRGV9, OR51E2, TFF3, SCHLAP1 and B3GNT6. In some embodiments, the at least nine genes are TMPRSS2-ERG, PCA3, PCAT14, ERG, TRGV9, OR51E2, TFF3, NKAIN1 and PCGEM1. In some embodiments, the at least nine genes are TMPRSS2-ERG, PCA3, PCAT14, ERG, TRGV9, OR51E2, TFF3, NKAIN1 and KLK4. In some embodiments, the at least nine genes awe TMPRSS2-ERG, PCA3, PCAT14, ERG, TRGV9, OR51E2, TFF3, NKAIN1 and B3GNT6. In some embodiments, the at least nine genes are TMPRSS2-ERG, PCA3, PCAT14, ERG, TRGV9, OR51E2, TFF3, NKAIN1 and SPON2. In some embodiments, the at least nine genes are TMPRSS2-ERG, PCA3, PCAT14, ERG, TRGV9, OR51E2, TFF3, NKAIN1 and TMSB15A. In some embodiments, the at least nine genes are TMPRSS2-ERG, PCA3, PCAT14, ERG, TRGV9, OR51E2, TFF3, CAMKK2 and PCGEM1. In some embodiments, the at least nine genes are TMPRSS2-ERG, PCA3, PCAT14, ERG, TRGV9, OR51E2, TFF3, CAMKK2 and B3GNT6. In some embodiments, the at least nine genes awe TMPRSS2-ERG, PCA3, PCAT14, ERG, TRGV9, OR51E2, TFF3, CAMKK2 and APOC1. In some embodiments, the at least nine genes awe TMPRSS2-ERG, PCA3, PCAT14, ERG, TRGV9, OR51E2, TFF3, CAMKK2 and HOXC6. In some embodiments, the at least nine genes awe TMPRSS2-ERG, PCA3, PCAT14, ERG, TRGV9, OR51E2, TFF3, CAMKK2 and SPON2. In some embodiments, the at least nine genes awe TMPRSS2-ERG, PCA3, PCAT14, ERG, TRGV9, OR51E2, TFF3, HOXC6 and PCGEM1. In some embodiments, the at least nine genes awe TMPRSS2-ERG, PCA3, PCAT14, ERG, TRGV9, OR51E2, TFF3, HOXC6 and TMSB15A. In some embodiments, the at least nine genes are TMPRSS2-ERG, PCA3, PCAT14, ERG, TRGV9, OR51E2, TFF3, HOXC6 and APOC1. In some embodiments, the at least nine genes are TMPRSS2-ERG, PCA3, PCAT14, ERG, TRGV9, OR51E2, TFF3, HOXC6 and KLK4. In some embodiments, the at least nine genes are TMPRSS2-ERG, PCA3, PCAT14, ERG, TRGV9, OR51E2, TFF3, SPON2 and APOC1. In some embodiments, the at least nine genes are TMPRSS2-ERG, PCA3, PCAT14, ERG, TRGV9, OR51E2, TFF3, SPON2 and PCGEM1. In some embodiments, the at least nine genes are TMPRSS2-ERG, PCA3, PCAT14, ERG, TRGV9, OR51E2, TFF3, SPON2 and B3GNT6. In some embodiments, the at least nine genes are TMPRSS2-ERG, PCA3, PCAT14, ERG, TRGV9, OR51E2, TFF3, TMSB15A and B3GNT6. In some embodiments, the at least nine genes are TMPRSS2-ERG, PCA3, PCAT14, ERG, TRGV9, OR51E2, TFF3, TMSB15A and KLK4. In some embodiments, the at least nine genes are TMPRSS2-ERG, PCA3, PCAT14, ERG, TRGV9, OR51E2, TFF3, TMSB15A and PCGEM1. In some embodiments, the at least nine genes are TMPRSS2-ERG, PCA3, PCAT14, ERG, TRGV9, OR51E2, TFF3, TMSB15A and APOC1. In some embodiments, the at least nine genes are TMPRSS2-ERG, PCA3, PCAT14, ERG, TRGV9, OR51E2, TFF3, APOC1 and KLK4. In some embodiments, the at least nine genes are TMPRSS2-ERG, PCA3, PCAT14, ERG, TRGV9, OR51E2, TFF3, APOC1 and PCGEM1. In some embodiments, the at least nine genes are TMPRSS2-ERG, PCA3, PCAT14, ERG, TRGV9, OR51E2, TFF3, KLK4 and PCGEM1. In some embodiments, the at least nine genes are TMPRSS2-ERG, PCA3, PCAT14, ERG, TRGV9, OR51E2, TFF3, B3GNT6 and PCGEM1.

In some embodiments, the methods described herein comprise detecting an amount of expression of at least ten genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least ten genes are TMPRSS2-ERG, PCA3, PCAT14, ERG, TRGV9, OR51E2, TFF3, SCHLAP1, HOXC6 and SPON2. In some embodiments, the at least ten genes are TMPRSS2-ERG, PCA3, PCAT14, ERG, TRGV9, OR51E2, TFF3, SCHLAP1, HOXC6 and PCGEM1. In some embodiments, the at least ten genes are TMPRSS2-ERG, PCA3, PCAT14, ERG, TRGV9, OR51E2, TFF3, SCHLAP1, NKAIN1 and PCGEM1. In some embodiments, the at least ten genes are TMPRSS2-ERG, PCA3, PCAT14, ERG, TRGV9, OR51E2, TFF3, SCHLAP1, CAMKK2 and PCGEM1. In some embodiments, the at least ten genes are TMPRSS2-ERG, PCA3, PCAT14, ERG, TRGV9, OR51E2, TFF3, SCHLAP1, HOXC6 and NKAIN1. In some embodiments, the at least ten genes are TMPRSS2-ERG, PCA3, PCAT14, ERG, TRGV9, OR51E2, TFF3, SCHLAP1, SPON2 and TMSB15A. In some embodiments, the at least ten genes are TMPRSS2-ERG, PCA3, PCAT14, ERG, TRGV9, OR51E2, TFF3, SCHLAP1, KLK4 and PCGEM1. In some embodiments, the at least ten genes are TMPRSS2-ERG, PCA3, PCAT14, ERG, TRGV9, OR51E2, TFF3, SCHLAP1, HOXC6 and CAMKK2. In some embodiments, the at least ten genes are TMPRSS2-ERG, PCA3, PCAT14, ERG, TRGV9, OR51E2, TFF3, SCHLAP1, TMBS15A and APOC1. In some embodiments, the at least ten genes awe TMPRSS2-ERG, PCA3, PCAT14, ERG, TRGV9, OR51E2, TFF3, SCHLAP1, B3GNT6 and PCGEM1. In some embodiments, the at least ten genes are TMPRSS2-ERG, PCA3, PCAT14, ERG, TRGV9, OR51E2, TFF3, SCHLAP1, HOXC6 and KLK4. In some embodiments, the at least ten genes are TMPRSS2-ERG, PCA3, PCAT14, ERG, TRGV9, OR51E2, TFF3, SCHLAP1, APOC1 and B3GNT6. In some embodiments, the at least ten genes are TMPRSS2-ERG, PCA3, PCAT14, ERG, TRGV9, OR51E2, TFF3, CAMKK2, NKAIN1 and PCGEM1. In some embodiments, the at least ten genes are TMPRSS2-ERG, PCA3, PCAT14, ERG, TRGV9, OR51E2, TFF3, CAMKK2, TMSB15A and KLK4. In some embodiments, the at least ten genes are TMPRSS2-ERG, PCA3, PCAT14, ERG, TRGV9, OR51E2, TFF3, CAMKK2, APOC1 and NKAIN1. In some embodiments, the at least ten genes awe TMPRSS2-ERG, PCA3, PCAT14, ERG, TRGV9, OR51E2, TFF3, CAMKK2, HOXC6 and SPON2. In some embodiments, the at least ten genes are TMPRSS2-ERG, PCA3, PCAT14, ERG, TRGV9, OR51E2, TFF3, KLK4, NKAIN1 and PCGEM1. In some embodiments, the at least ten genes are TMPRSS2-ERG, PCA3, PCAT14, ERG, TRGV9, OR51E2, TFF3, KLK4, SPON2 and B3GNT6. In some embodiments, the at least ten genes awe TMPRSS2-ERG, PCA3, PCAT14, ERG, TRGV9, OR51E2, TFF3, HOXC6, SPON2 and PCGEM1. In some embodiments, the at least ten genes are TMPRSS2-ERG, PCA3, PCAT14, ERG, TRGV9, OR51E2, TFF3, HOXC6, TMSB15A and APCOC1. In some embodiments, the at least ten genes are TMPRSS2-ERG, PCA3, PCAT14, ERG, TRGV9, OR51E2, TFF3, HOXC6, SPON2 and NKAIN1. In some embodiments, the at least ten genes awe TMPRSS2-ERG, PCA3, PCAT14, ERG, TRGV9, OR51E2, TFF3, HOXC6, APOC1 and B3GNT6. In some embodiments, the at least ten genes are TMPRSS2-ERG, PCA3, PCAT14, ERG, TRGV9, OR51E2, TFF3, SPON2, APOC1 and B3GNT6. In some embodiments, the at least ten genes awe TMPRSS2-ERG, PCA3, PCAT14, ERG, TRGV9, OR51E2, TFF3, SPON2, TMSB15A and PCGEM1. In some embodiments, the at least ten genes are TMPRSS2-ERG, PCA3, PCAT14, ERG, TRGV9, OR51E2, TFF3, SPON2, TMSB15A and NKAIN1. In some embodiments, the at least ten genes awe TMPRSS2-ERG, PCA3, PCAT14, ERG, TRGV9, OR51E2, TFF3, B3GNT6, NKAIN1 and PCGEM1. In some embodiments, the at least ten genes are TMPRSS2-ERG, PCA3, PCAT14, ERG, TRGV9, OR51E2, TFF3, B3GNT6, APOC1 and TMSB15A. In some embodiments, the at least ten genes are TMPRSS2-ERG, PCA3, PCAT14, ERG, TRGV9, OR51E2, TFF3, APOC1, NKAIN1 and PCGEM1. In some embodiments, the at least ten genes are TMPRSS2-ERG, PCA3, PCAT14, ERG, TRGV9, OR51E2, TFF3, APOC1, TMSB15A and PCGEM1. In some embodiments, the at least ten genes are TMPRSS2-ERG, PCA3, PCAT14, ERG, TRGV9, OR51E2, TFF3, TMSB15A, NKAIN1 and PCGEM1.

In some embodiments, the methods described herein comprise detecting an amount of expression of at least eleven genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least eleven genes awe TMPRSS2-ERG, PCA3, PCAT14, ERG, TRGV9, OR51E2, TFF3, SCHLAP1, HOXC6, SPON2, and TMSB15A. In some embodiments, the at least eleven genes are TMPRSS2-ERG, PCA3, PCAT14, ERG, TRGV9, OR51E2, TFF3, SCHLAP1, HOXC6, SPON2 and PCGEM1. In some embodiments, the at least eleven genes are TMPRSS2-ERG, PCA3, PCAT14, ERG, TRGV9, OR51E2, TFF3, SCHLAP1, HOXC6, NKAIN1 and PCGEM1. In some embodiments, the at least eleven genes are TMPRSS2-ERG, PCA3, PCAT14, ERG, TRGV9, OR51E2, TFF3, SCHLAP1, HOXC6, B3GNT6 and PCGEM1. In some embodiments, the at least eleven genes are TMPRSS2-ERG, PCA3, PCAT14, ERG, TRGV9, OR51E2, TFF3, SCHLAP1, HOXC6, SPON2 and KLK4. In some embodiments, the at least eleven genes are TMPRSS2-ERG, PCA3, PCAT14, ERG, TRGV9, OR51E2, TFF3, SCHLAP1, CAMKK2, NKAIN1 and PCGEM1. In some embodiments, the at least eleven genes awe TMPRSS2-ERG, PCA3, PCAT14, ERG, TRGV9, OR51E2, TFF3, SCHLAP1, CAMKK2, KLK4 and NKAIN1. In some embodiments, the at least eleven genes are TMPRSS2-ERG, PCA3, PCAT14, ERG, TRGV9, OR51E2, TFF3, SCHLAP1, CAMKK2, B3GNT6 and KLK4. In some embodiments, the at least eleven genes are TMPRSS2-ERG, PCA3, PCAT14, ERG, TRGV9, OR51E2, TFF3, SCHLAP1, CAMKK2, SPON2 and TMSB15A. In some embodiments, the at least eleven genes are TMPRSS2-ERG, PCA3, PCAT14, ERG, TRGV9, OR51E2, TFF3, SCHLAP1, APOC1, B3GNT6 and KLK4. In some embodiments, the at least eleven genes are TMPRSS2-ERG, PCA3, PCAT14, ERG, TRGV9, OR51E2, TFF3, SCHLAP1, APOC1, NKAIN1 and PCGEM1. In some embodiments, the at least eleven genes are TMPRSS2-ERG, PCA3, PCAT14, ERG, TRGV9, OR51E2, TFF3, SCHLAP1, APOC1, TMSB15A and B3GNT6. In some embodiments, the at least eleven genes are TMPRSS2-ERG, PCA3, PCAT14, ERG, TRGV9, OR51E2, TFF3, SCHLAP1, APOC1, SPON2 and TMSB15A. In some embodiments, the at least eleven genes are TMPRSS2-ERG, PCA3, PCAT14, ERG, TRGV9, OR51E2, TFF3, SCHLAP1, APOC1, TMSB15A and NKAIN1. In some embodiments, the at least eleven genes are TMPRSS2-ERG, PCA3, PCAT14, ERG, TRGV9, OR51E2, TFF3, SCHLAP1, APOC1, B3GNT6 and PCGEM1. In some embodiments, the at least eleven genes are TMPRSS2-ERG, PCA3, PCAT14, ERG, TRGV9, OR51E2, TFF3, SCHLAP1, TMSB15A, KLK4 and PCGEM1.

In some embodiments, the methods described herein comprise detecting an amount of expression of at least twelve genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least twelve genes are TMPRSS2-ERG, PCA3, PCAT14, ERG, TRGV9, OR51E2, TFF3, SCHLAP1, HOXC6, CAMKK2, NKAIN1 and PCGEM1. In some embodiments, the at least twelve genes are TMPRSS2-ERG, PCA3, PCAT14, ERG, TRGV9, OR51E2, TFF3, SCHLAP1, HOXC6, CAMKK2, B3GNT6 and KLK4. In some embodiments, the at least twelve genes are TMPRSS2-ERG, PCA3, PCAT14, ERG, TRGV9, OR51E2, TFF3, SCHLAP1, HOXC6, CAMKK2, KLK$ and NKAIN1. In some embodiments, the at least twelve genes are TMPRSS2-ERG, PCA3, PCAT14, ERG, TRGV9, OR51E2, TFF3, SCHLAP1, HOXC6, CAMKK2, SPON2 and PCGEM1. In some embodiments, the at least twelve genes are TMPRSS2-ERG, PCA3, PCAT14, ERG, TRGV9, OR51E2, TFF3, SCHLAP1, HOXC6, CAMKK2, SPON2 and TMSB15A. In some embodiments, the at least twelve genes are TMPRSS2-ERG, PCA3, PCAT14, ERG, TRGV9, OR51E2, TFF3, SCHLAP1, HOXC6, SPON2, NKAIN1 and PCGEM1. In some embodiments, the at least twelve genes are TMPRSS2-ERG, PCA3, PCAT14, ERG, TRGV9, OR51E2, TFF3, SCHLAP1, HOXC6, SPON2, TMSB15A and PCGEM1. In some embodiments, the at least twelve genes are TMPRSS2-ERG, PCA3, PCAT14, ERG, TRGV9, OR51E2, TFF3, SCHLAP1, HOXC6, SPON2, TMSB15A and APOC1. In some embodiments, the at least twelve genes are TMPRSS2-ERG, PCA3, PCAT14, ERG, TRGV9, OR51E2, TFF3, SCHLAP1, HOXC6, SPON2, TMSB15A and NKAIN1. In some embodiments, the at least twelve genes are TMPRSS2-ERG, PCA3, PCAT14, ERG, TRGV9, OR51E2, TFF3, SCHLAP1, HOXC6, SPON2, APOC1 and B3GNT6. In some embodiments, the at least twelve genes are TMPRSS2-ERG, PCA3, PCAT14, ERG, TRGV9, OR51E2, TFF3, SCHLAP1, HOXC6, SPON2, KLK4 and PCGEM1. In some embodiments, the at least twelve genes are TMPRSS2-ERG, PCA3, PCAT14, ERG, TRGV9, OR51E2, TFF3, SCHLAP1, HOXC6, TMSB15A, APOC1 and B3GNT6. In some embodiments, the at least twelve genes are TMPRSS2-ERG, PCA3, PCAT14, ERG, TRGV9, OR51E2, TFF3, SCHLAP1, HOXC6, TMSB15A, APOC1 and PCGEM1. In some embodiments, the at least twelve genes are TMPRSS2-ERG, PCA3, PCAT14, ERG, TRGV9, OR51E2, TFF3, SCHLAP1, HOXC6, TMSB15A, B3GNT6 and KLK4. In some embodiments, the at least twelve genes are TMPRSS2-ERG, PCA3, PCAT14, ERG, TRGV9, OR51E2, TFF3, SCHLAP1, HOXC6, TMSB15A, APOC1 and NKAIN1. In some embodiments, the at least twelve genes are TMPRSS2-ERG, PCA3, PCAT14, ERG, TRGV9, OR51E2, TFF3, SCHLAP1, HOXC6, APOC1, B3GNT6 and KLK4. In some embodiments, the at least twelve genes are TMPRSS2-ERG, PCA3, PCAT14, ERG, TRGV9, OR51E2, TFF3, SCHLAP1, HOXC6, KLK4, NKAIN1 and PCGEM1. In some embodiments, the at least twelve genes are TMPRSS2-ERG, PCA3, PCAT14, ERG, TRGV9, OR51E2, TFF3, SCHLAP1, HOXC6, B3GNT6, NKAIN1 and PCGEM1. In some embodiments, the at least twelve genes are TMPRSS2-ERG, PCA3, PCAT14, ERG, TRGV9, OR51E2, TFF3, SCHLAP1, HOXC6, APOC1, B3GNT6 and PCGEM1.

In some embodiments, the methods described herein comprise detecting an amount of expression of at least thirteen genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least thirteen genes are TMPRSS2-ERG, PCA3, PCAT14, ERG, TRGV9, OR51E2, TFF3, SCHLAP1, HOXC6, SPON2, TMSB15A, APOC1 and B3GNT6. In some embodiments, the at least thirteen genes awe TMPRSS2-ERG, PCA3, PCAT14, ERG, TRGV9, OR51E2, TFF3, SCHLAP1, HOXC6, SPON2, TMSB15A, NKAIN1 and PCGEM1. In some embodiments, the at least thirteen genes awe TMPRSS2-ERG, PCA3, PCAT14, ERG, TRGV9, OR51E2, TFF3, SCHLAP1, HOXC6, SPON2, TMSB15A, KLK4 and PCGEM1. In some embodiments, the at least thirteen genes are TMPRSS2-ERG, PCA3, PCAT14, ERG, TRGV9, OR51E2, TFF3, SCHLAP1, HOXC6, SPON2, TMSB15A, APOC1 and CAMKK2. In some embodiments, the at least thirteen genes are TMPRSS2-ERG, PCA3, PCAT14, ERG, TRGV9, OR51E2, TFF3, SCHLAP1, HOXC6, SPON2, TMSB15A, KLK4 and CAMKK2. In some embodiments, the at least thirteen genes are TMPRSS2-ERG, PCA3, PCAT14, ERG, TRGV9, OR51E2, TFF3, SCHLAP1, HOXC6, TMSB15A, APOC1, B3GNT6 and KLK4. In some embodiments, the at least thirteen genes are TMPRSS2-ERG, PCA3, PCAT14, ERG, TRGV9, OR51E2, TFF3, SCHLAP1, HOXC6, TMSB15A, APOC1, B3GNT6 and NKAIN1. In some embodiments, the at least thirteen genes are TMPRSS2-ERG, PCA3, PCAT14, ERG, TRGV9, OR51E2, TFF3, SCHLAP1, HOXC6, TMSB15A, APOC1, CAMKK2 and NKAIN1. In some embodiments, the at least thirteen genes are TMPRSS2-ERG, PCA3, PCAT14, ERG, TRGV9, OR51E2, TFF3, SCHLAP1, HOXC6, APOC1, B3GNT6, KLK4 and CAMKK2. In some embodiments, the at least thirteen genes are TMPRSS2-ERG, PCA3, PCAT14, ERG, TRGV9, OR51E2, TFF3, SCHLAP1, HOXC6, APOC1, B3GNT6, KLK4 and PGEM1. In some embodiments, the at least thirteen genes awe TMPRSS2-ERG, PCA3, PCAT14, ERG, TRGV9, OR51E2, TFF3, SCHLAP1, HOXC6, APOC1, B3GNT6, NKAIN1 and PCGEM1. In some embodiments, the at least thirteen genes awe TMPRSS2-ERG, PCA3, PCAT14, ERG, TRGV9, OR51E2, TFF3, SCHLAP1, HOXC6, B3GNT6, KLK4, NKAIN1 and CAMKK2. In some embodiments, the at least thirteen genes awe TMPRSS2-ERG, PCA3, PCAT14, ERG, TRGV9, OR51E2, TFF3, SCHLAP1, HOXC6, KLK4, CAMKK2, NKAIN1 and PCGEM1. In some embodiments, the at least thirteen genes are TMPRSS2-ERG, PCA3, PCAT14, ERG, TRGV9, OR51E2, TFF3, SCHLAP1, HOXC6, SPON2, CAMKK2, NKAIN1 and PCGEM1. In some embodiments, the at least thirteen genes are TMPRSS2-ERG, PCA3, PCAT14, ERG, TRGV9, OR51E2, TFF3, SCHLAP1, HOXC6, APOC1, CAMKK2, NKAIN1, and PCGEM1. In some embodiments, the at least thirteen genes are TMPRSS2-ERG, PCA3, PCAT14, ERG, TRGV9, OR51E2, TFF3, SCHLAP1, HOXC6, SPON2, B3GNT6, NKAIN1, and PCGEM1. In some embodiments, the at least thirteen genes are TMPRSS2-ERG, PCA3, PCAT14, ERG, TRGV9, OR51E2, TFF3, SCHLAP1, HOXC6, SPON2, B3GNT6, KLK4 and CAMKK2. In some embodiments, the at least thirteen genes are TMPRSS2-ERG, PCA3, PCAT14, ERG, TRGV9, OR51E2, TFF3, SCHLAP1, HOXC6, SPON2, B3GNT6, KLK$ and PCGEM1. In some embodiments, the at least thirteen genes are TMPRSS2-ERG, PCA3, PCAT14, ERG, TRGV9, OR51E2, TFF3, SCHLAP1, HOXC6, SPON2, APOC1, KLK4 and NKAIN1. In some embodiments, the at least thirteen genes are TMPRSS2-ERG, PCA3, PCAT14, ERG, TRGV9, OR51E2, TFF3, SCHLAP1, HOXC6, TMSB15A, B3GNT6, CAMKK2 and PCGEM1.

In some embodiments, the methods described herein comprise detecting an amount of expression of at least fourteen genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least fourteen genes are TMPRSS2-ERG, PCA3, PCAT14, ERG, TRGV9, OR51E2, TFF3, SCHLAP1, HOXC6, PCGEM1, B3GNT6, KLK4, CAMKK2, and NKAIN1. In some embodiments, the at least fourteen genes are TMPRSS2-ERG, PCA3, PCAT14, ERG, TRGV9, OR51E2, TFF3, SCHLAP1, HOXC6, PCGEM1, SPON2, KLK4, CAMKK2 and NKAIN1. In some embodiments, the at least fourteen genes are TMPRSS2-ERG, PCA3, PCAT14, ERG, TRGV9, OR51E2, TFF3, SCHLAP1, HOXC6, PCGEM1, SPON2, TMSB15A, CAMKK2 and NKAIN1. In some embodiments, the at least fourteen genes are TMPRSS2-ERG, PCA3, PCAT14, ERG, TRGV9, OR51E2, TFF3, SCHLAP1, HOXC6, PCGEM1, SPON2, TMSB15A, APOC1 and NKAIN1. In some embodiments, the at least fourteen genes are TMPRSS2-ERG, PCA3, PCAT14, ERG, TRGV9, OR51E2, TFF3, SCHLAP1, HOXC6, PCGEM1, SPON2, TMSB15A, APOC1, and B3GNT6. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, KLK4 and APOC1. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, B3GNT6 and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, KLK4, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, B3GNT6 and APOC1. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, KLK4, B3GNT6 and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, KLK4, TMSB15A and SPON1. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, B3GNT6, APOC1 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, KLK4, B3GNT6, APOC1 and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, KLK4, B3GNT6 and APOC1. In some embodiments, the at least fourteen genes awe TMPRSS2-ERG, PCA3, PCAT14, ERG, TRGV9, OR51E2, TFF3, SCHLAP1, HOXC6, PCGEM1, CAMKK2, KLK4, B3GNT6, and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, APOC1 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, B3GNT6, APOC1 and SPON2. In some embodiments, the at least fourteen genes are TMPRSS2-ERG, PCA3, PCAT14, ERG, TRGV9, OR51E2, TFF3, SCHLAP1, HOXC6, PCGEM1, KLK4, B3GNT6, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, KLK4, B3GNT6 and APOC). In some embodiments, the at least fourteen genes awe TMPRSS2-ERG, PCA3, PCAT14, ERG, TRGV9, OR51E2, TFF3, SCHLAP1, HOXC6, PCGEM1, NKAIN1, CAMKK2, KLK4 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, APOC1 and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, B3GNT6, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, B3GNT6, and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, KLK4, APOC1 and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, B3GNT6, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, B3GNT6, APOC1 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, KLK4, B3GNT6 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, KLK4, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, NKAIN1, B3GNT6, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, NKAIN1, CAMKK2, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, NKAIN1, CAMKK2, KLK4, TMSB15A and SPON2. In some embodiments, the at least fourteen genes are TMPRSS2-ERG, PCA3, PCAT14, ERG, TRGV9, OR51E2, TFF3, SCHLAP1, HOXC6, NKAIN1, CAMKK2, KLK4, B3GNT6 and SPON2. In some embodiments, the at least fourteen genes are TMPRSS2-ERG, PCA3, PCAT14, ERG, TRGV9, OR51E2, TFF3, SCHLAP1, HOXC6, NKAIN1, CAMKK2, KLK4, APOC1 and SPON2. In some embodiments, the at least fourteen genes are TMPRSS2-ERG, PCA3, PCAT14, ERG, TRGV9, OR51E2, TFF3, SCHLAP1, HOXC6, NKAIN1, KLK4, B3GNT6, APOC1 and SPON2. In some embodiments, the at least fourteen genes are TMPRSS2-ERG, PCA3, PCAT14, ERG, TRGV9, OR51E2, TFF3, SCHLAP1, HOXC6, NKAIN1, KLK4, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, KLK4, B3GNT6, APOC1, TMSB15A and SPON2.

In some embodiments, the methods described herein comprise detecting an amount of expression of at least fifteen genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least fifteen genes are TMPRSS2-ERG, PCA3, PCAT14, ERG, TRGV9, OR51E2, TFF3, SCHLAP1, HOXC6, SPON2, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4 and CAMKK2. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4, and NKAIN1. In some embodiments, the at least fifteen genes are TMPRSS2-ERG, PCA3, PCAT14, ERG, TRGV9, OR51E2, TFF3, SCHLAP1, HOXC6, SPON2, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, CAMKK2 and NKAIN1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, KLK4, CAMKK2 and NKAIN1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, CAMKK2 and NKAIN1. In some embodiments, the at least fifteen genes are TMPRSS2-ERG, PCA3, PCAT14, ERG, TRGV9, OR51E2, TFF3, SCHLAP1, TMSB15A, APOC1, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, APOC1, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, APOC1, B3GNT6, KLK4, CAMKK2, NKAIN1 an PCGEM1. In some embodiments, the at least fifteen genes are TMPRSS2-ERG, PCA3, PCAT14, ERG, TRGV9, OR51E2, TFF3, SCHLAP1, HOXC6, TMSB15A, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3GNT6, KLK4, CAMKK2 and NKAIN1. In some embodiments, the at least fifteen genes are TMPRSS2-ERG, PCA3, PCAT14, ERG, TRGV9, OR51E2, TFF3, SCHLAP1, SPON2, TMSB15A, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, CAMKK2, NKAIN1 and PCGEM1. In some embodiments, the at least fifteen genes awe TMPRSS2-ERG, PCA3, PCAT14, ERG, TRGV9, OR51E2, TFF3, SCHLAP1, HOXC6, SPON2, APOC1, B3GNT6, KLK4, CAMKK2 and NKAIN1. In some embodiments, the at least fifteen genes awe TMPRSS2-ERG, PCA3, PCAT14, ERG, TRGV9, OR51E2, TFF3, SCHLAP1, HOXC6, SPON2, APOC1, B3GNT6, KLK4, NKAIN1 and PCGEM1. In some embodiments, the at least fifteen genes awe TMPRSS2-ERG, PCA3, PCAT14, ERG, TRGV9, OR51E2, TFF3, SCHLAP1, HOXC6, SPON2, APOC1, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, KLK4, NKAIN1 and PCGEM1. In some embodiments, the at least fifteen genes awe TMPRSS2-ERG, PCA3, PCAT14, ERG, TRGV9, OR51E2, TFF3, SCHLAP1, HOXC6, TMSB15A, APOC1, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, KLK$, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, KLK4, CAMKK2, NKAIN1 and PCGEM1. In some embodiments, the at least fifteen genes awe TMPRSS2-ERG, PCA3, PCAT14, ERG, TRGV9, OR51E2, TFF3, SCHLAP1, HOXC6, TMSB15A, APOC1, B3GNT6, KLK4, CAMKK2 and NKAIN1. In some embodiments, the at least fifteen genes awe TMPRSS2-ERG, PCA3, PCAT14, ERG, TRGV9, OR51E2, TFF3, SCHLAP1, HOXC6, SPON2, APOC1, B3GNT6, KLK4, CAMKK2 and PCGEM1. In some embodiments, the at least fifteen genes awe TMPRSS2-ERG, PCA3, PCAT14, ERG, TRGV9, OR51E2, TFF3, SCHLAP1, HOXC6, SPON2, APOC1, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, CAMKK2 and NKAIN1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3NGTZ6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, B3GNT6, KLK4, CAMKK2, NKAIN1 and PCGEM1.

In some embodiments, the methods described herein comprise detecting an amount of expression of at least sixteen genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4, CAMKK2 and NKAIN1. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4, CAMKK2 and PCGEM1. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, B3GNT6, CAMKK2, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, KLK4, CAMKK2, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, SPON2, TMSB15A, B3GNT6, KLK4, CAMKK2, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, SPON2, APOC1, B3GNT6, KLK4, CAMKK2, NKAIN1 and PCGEM1. In some embodiments, the at least sixteen genes awe TMPRSS2-ERG, PCA3, PCAT14, ERG, TRGV9, OR51E2, TFF3, SCHLAP1, HOXC6, TMSB15A, APOC1, B3GNT6, KLK4, CAMKK2, NKAIN1 and PCGEM1. In some embodiments, the at least sixteen genes awe TMPRSS2-ERG, PCA3, PCAT14, ERG, TRGV9, OR51E2, TFF3, SCHLAP1, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, TRGV9, OR51E2, TFF3, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, TRGV9, OR51E2, SCHLAP1, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, TRGV9, TFF3, SCHLAP1, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, OR51E2, TFF3, SCHLAP1, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, TRGV9, OR51E2, TFF3, SCHLAP1, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, ERG, TRGV9, OR51E2, TFF3, SCHLAP1, HOXC6, SPON2, TMSB15A, APOC1, B3GNT6, KLK4, CAMKK2, NKAIN1 and PCGEM1.

In some embodiments, the methods described herein comprise detecting an amount of expression of each of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

### Detecting Expression of Genes

In some embodiments, detecting an amount of expression of each of one or more of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 comprises detecting a nucleic acid. In some embodiments, detecting an amount of expression of each of one or more of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 comprises detecting an mRNA.

In some embodiments, detecting an amount of expression of each of one or more of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 comprises detecting a protein.

The amount of expression of each of the one or more genes can be detected using any of a variety of nucleic acid techniques, including but not limited to: nucleic acid sequencing; nucleic acid hybridization; and nucleic acid amplification.

The amount of gene expression can be detected using a Second Generation (i.e., Next Generation or Next-Gen), Third Generation (i.e., Next-Next-Gen), or Fourth Generation (i.e., N3-Gen) sequencing technology including, but not limited to, pyrosequencing, sequencing-by-ligation, single molecule sequencing, sequence-by-synthesis (SBS), sequencing by expansion (SBX), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008). Those of skill in the art will recognize that because RNA is less stable in the cell and more prone to nuclease attack experimentally RNA can be reverse transcribed to DNA before sequencing.

A number of DNA sequencing techniques are suitable for gene expression detection, including fluorescence-based sequencing methodologies (See, e.g., Birren et al., Genome Analysis: Analyzing DNA, 1, Cold Spring Harbor, N.Y. In some embodiments, the sequencing is automated sequencing techniques understood in the art. In some embodiments, the sequencing is parallel sequencing of partitioned amplicons (PCT Publication No: WO2006084132 to Kevin McKernan et al. In some embodiments, the sequencing is DNA sequencing by parallel oligonucleotide extension (See, e.g., U.S. Pat. No. 5,750,341 to Macevicz et al., and U.S. Pat. No. 6,306,597 to Macevicz et al. Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803, the 454 picotiter pyrosequencing technology (Margulies et al., 2005 Nature 437, 376-380; US 20050130173, the Solexa single base addition technology (Bennett et al., 2005, Pharmacogenomics, 6, 373-382; U.S. Pat. Nos. 6,787,308; 6,833,246, the Lynx massively parallel signature sequencing technology (Brenner et al. (2000). Nat. Biotechnol. 18:630-634; U.S. Pat. Nos. 5,695,934; 5,714,330, and the Adessi PCR colony technology (Adessi et al. (2000). Nucleic Acid Res. 28, E87; WO 00018957.

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH can be used to measure and localize mRNAs and other transcripts (e.g., cancer markers) within tissue sections or whole mounts. Sample cells and tissues can be treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Expression of each of the one or more genes of the present methods can be detected by conducting one or more hybridization reactions. The one or more hybridization reactions can comprise one or more hybridization arrays, hybridization reactions, hybridization chain reactions, isothermal hybridization reactions, nucleic acid hybridization reactions, or a combination thereof. The one or more hybridization arrays can comprise hybridization array genotyping, hybridization array proportional sensing, DNA hybridization arrays, macroarrays, microarrays, high-density oligonucleotide arrays, genomic hybridization arrays, comparative hybridization arrays, or a combination thereof.

Microarrays including, but not limited to, DNA microarrays (e.g., cDNA microarrays and oligonucleotide microarrays); protein microarrays; tissue microarrays; transfection or cell microarrays; chemical compound microarrays; and antibody microarrays, can optionally be employed. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., cancer markers) by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink-jet printing; or, electrochemistry on microelectrode arrays.

Detection of an amount of expression of the one or more genes of the present methods can comprise conducting one or more amplification reactions. Nucleic acids (e.g., cancer markers) can be amplified prior to or simultaneous with detection. Conducting one or more amplification reactions can comprise one or more PCR-based amplifications, non-PCR based amplifications, or a combination thereof. Illustrative non-limiting examples of nucleic acid amplification techniques include, but are not limited to, polymerase chain reaction (PCR), quantitative polymerase chain reaction (qPCR), digital polymerase chain reaction (dPCR), reverse transcription polymerase chain reaction (RT-PCR), nested PCR, linear amplification, multiple displacement amplification (MDA), real-time SDA, rolling circle amplification, circle-to-circle amplification transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and Murakawa et al., DNA 7: 287 (1988).

Transcription mediated amplification (U.S. Pat. Nos. 5,480,784 and 5,399,491), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518. In a variation described in U.S. Publ. No. 20060046265, TMA optionally incorporates the use of blocking moieties, terminating moieties, and other modifying moieties to improve TMA process sensitivity and accuracy.

The ligase chain reaction (Weiss, R., Science 254: 1292 (1991), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad Sci. USA 89: 392-396 (1992); U.S. Pat. Nos. 5,270,184 and 5,455,166), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence-based amplification (U.S. Pat. No. 5,130,238), commonly referred to as NASBA; one that uses an RNA replicase to amplify the probe molecule itself (Lizardi et al., BioTechnol. 6: 1197 (1988)), commonly referred to as Qβ replicase; a transcription-based amplification method (Kwoh et al., Proc. Natl. Acad. Sci. USA 86:1173 (1989)); and, self-sustained sequence replication (Guatelli et al., Proc. Natl. Acad Sci. USA 87: 1874 (1990)). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, DC (1993)).

In some embodiments, amplification methods are real time quantitative PCR methods (QPCR). A real-time polymerase chain reaction (real-time PCR, or qPCR) is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively (quantitative real-time PCR) and semi-quantitatively (i.e., above/below a certain amount of DNA molecules) (semi-quantitative real-time PCR). Two common methods for the detection of PCR products in real-time PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

In some embodiments, detection of an amount of gene expression comprises detecting a protein. Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldifluoride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Immuno-polymerase chain reaction (IPCR) utilizes nucleic acid amplification techniques to increase signal generation in antibody-based immunoassays. In some embodiments, signal amplification is used in antibody-based immunoassays to increase detection sensitivity. The target proteins are bound to antibodies which are directly or indirectly conjugated to oligonucleotides. Unbound antibodies are washed away, and the remaining bound antibodies have their oligonucleotides amplified. Protein detection occurs via detection of amplified oligonucleotides using standard nucleic acid detection methods, including real-time methods.

In some embodiments, detecting an amount of gene expression comprises detecting mRNA. In some embodiments, the amount of mRNA is detected using RT-qPCR analysis which provides Ct (cycle threshold values) for each mRNA detected. In a real-time PCR assay a positive reaction is detected by accumulation of a fluorescent signal. The Ct value is defined as the number of cycles required for the fluorescent signal to cross the threshold (i.e., exceeds the background level). Ct levels are inversely proportional to the amount of target nucleic acid in the sample (i.e., the lower the Ct value the greater the amount of mRNA in the sample).

In some embodiments, the amount of expression of any one of the genes described herein is normalized to an amount of expression of a reference gene. In some embodiments, the amount of expression of mRNA is normalized to an amount of expression of mRNA of a reference gene. Reference genes suitable for normalization are known to those of skill in the art and include, but are not limited to, KLK3, CYPB561A3, EEF1A2, GAPDH, HPN, KLK2, LBH, NUDT8, SPDEF, or TRGV. In some embodiments, the reference gene is KLK3.

Compositions that are useful for detecting an amount of gene expression can comprise one or more antibodies, probes, amplification oligonucleotides or reagents.

The compositions can comprise 1 or more, 2 or more, 3 or more, or 4 or more antibodies, probes, pairs of probes, pairs of amplification oligonucleotide, or sequencing primers.

The probes or primers can hybridize to 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, or 21 or more target molecules. The target molecules may be RNA, DNA, cDNA, mRNA, a portion or fragment thereof or a combination thereof. In some instances, at least a portion of the target molecules are cancer markers. The probes may hybridize to 1 or more, or 2 or more cancer markers disclosed herein.

Typically, the probes or primers comprise a target specific sequence. The target specific sequence may be complementary to at least a portion of the target molecule. The target specific sequence may be at least about 50% or more, 55% or more, 60% or more, 65% or more, 70% or more, 75% or more, 80% or more, 85% or more, 90% or more, 95% or more, 97% or more, 98% or more, or 100% complementary to at least a portion of the target molecule.

The target specific sequence can be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more nucleotides in length. In some instances, the target specific sequence is between about 8 to about 20 nucleotides, 10 to about 18 nucleotides, or 12 to about 16 nucleotides in length.

These compositions can comprise a plurality of probes or primers, wherein the two or more probes of the plurality of probes comprise identical target specific sequences. The compositions may comprise a plurality of probes, wherein the two or more probes of the plurality of probes comprise different target specific sequences.

The probes can further comprise a unique sequence. The unique sequence is noncomplementary to the cancer marker. The unique sequence may comprise a label, barcode, or unique identifier. The unique sequence may comprise a random sequence, nonrandom sequence, or a combination thereof. The unique sequence may be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 22 or more, 24 or more, 26 or more, 28 or more, 30 or more nucleotides in length. In some instances, the unique sequence is between about 8 to about 20 nucleotides, 10 to about 18 nucleotides, or 12 to about 16 nucleotides in length.

The probes can further comprise a universal sequence. The universal sequence may comprise a primer binding site. The universal sequence may enable detection of the target sequence. The universal sequence may enable amplification of the target sequence. The universal sequence may enable transcription or reverse transcription of the target sequence. The universal sequence may enable sequencing of the target sequence.

Compositions comprising a probe or primer can be provided on a solid support. The solid support can comprise one or more beads, plates, solid surfaces, wells, chips, or a combination thereof. The beads can be magnetic, antibody coated, protein A crosslinked, protein G crosslinked, streptavidin coated, oligonucleotide conjugated, silica coated, or a combination thereof. Examples of beads include, but are not limited to, Ampure beads, AMPure XP beads, streptavidin beads, agarose beads, magnetic beads, Dynabeads®, MACS® microbeads, antibody conjugated beads (e.g., anti-immunoglobulin microbead), protein A conjugated beads, protein G conjugated beads, protein A/G conjugated beads, protein L conjugated beads, oligo-dT conjugated beads, silica beads, silica-like beads, anti-biotin microbead, anti-fluorochrome microbead, and BcMag™ Carboxy-Terminated Magnetic Beads.

The compositions can comprise one or more primers or primer pairs capable of amplifying target molecules, or fragments or subsequences or complements thereof. The nucleotide sequences of the target molecules may be provided in computer-readable media for in silico applications and as a basis for the design of appropriate primers for amplification of one or more target molecules.

Primers based on the nucleotide sequences of target molecules can be designed for use in amplification of the target molecules. For use in amplification reactions such as PCR, a pair of primers can be used. The exact composition of the primer sequences is not critical to the disclosure, but for most applications the primers may hybridize to specific sequences of the target molecules or the universal sequence of the probe under stringent conditions, particularly under conditions of high stringency, as known in the art. The pairs of primers are usually chosen so as to generate an amplification product of at least about 15 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 450 or more, 500 or more, 600 or more, 700 or more, 800 or more, 900 or more, or 1000 or more nucleotides. Algorithms for the selection of primer sequences are generally known and are commercially available. These primers may be used in standard quantitative or qualitative PCR-based assays to assess transcript expression levels of target molecules. Alternatively, these primers may be used in combination with probes, such as molecular beacons in amplifications using real-time PCR.

The nucleotide sequence of the entire length of the primer does not need to be derived from the target sequence. Thus, for example, the primer may comprise nucleotide sequences at the 5′ and/or 3′ termini that are not derived from the target molecule. Nucleotide sequences which are not derived from the nucleotide sequence of the target molecule may provide additional functionality to the primer. For example, they may provide a restriction enzyme recognition sequence or a “tag” that facilitates detection, isolation, purification or immobilization onto a solid support. Alternatively, the additional nucleotides may provide a self-complementary sequence that allows the primer to adopt a hairpin configuration. Such configurations may be necessary for certain primers, for example, molecular beacon and Scorpion primers, which can be used in solution hybridization techniques.

The probes or primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target molecule is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer to allow detection and/or quantitation of a target polynucleotide representing the target molecule of interest. The target polynucleotide may be the expressed target molecule RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different target molecules may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g., biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the target molecule. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a target polynucleotide may comprise a biotin-binding species, and an optically detectable label may be conjugated to biotin and then bound to the labeled target polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Coding schemes may optionally be used, comprising encoded particles and/or encoded tags associated with different polynucleotides of the disclosure. A variety of different coding schemes are known in the art, including fluorophores, including SCNCs, deposited metals, and RF tags.

### Subjects and Samples

In some aspects, the present methods comprise detecting an amount of expression of each of one or more of the genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample from a subject, e.g., a urine sample. In some embodiments, a subject from whom a sample is obtained can be selected by the skilled practitioner. In some embodiments, selection of the subject is based upon consideration or analysis of one or more factors. Such factors for consideration include, but are not limited to, family history of a specific disease, genetic predisposition for the disease, increased risk for the disease, physical symptoms which indicate the disease, or environmental reasons. Environmental reasons can include, but are not limited to, lifestyle or exposure to agents which cause or contribute to the specific disease. In some embodiments, selection of a subject is based on the subject's previous history with the disease, positive diagnosis prior to therapy or after therapy, treatment for the disease, or remission or recovery from the disease.

In some embodiments, samples for use in the present methods comprise a nucleic acid, and the detecting comprises detecting an amount of the nucleic acid. In some embodiments, samples for use in the present methods comprise a protein, and the detecting comprises detecting an amount of the protein. In some embodiments, samples for use in the present methods comprise mRNA, and the detecting comprises detecting an amount of the mRNA.

In some embodiments, the sample is any source of biological material, including cells, tissue, secretions, or fluid, e.g., bodily fluids. Non-limiting examples of the source of the sample include an aspirate, a needle biopsy, a cytology pellet, a bulk tissue preparation or a section thereof (e.g., obtained by surgery, biopsy, or autopsy), lymph fluid, blood, plasma, serum, tumors, and organs. In some embodiments, the sample is urine, semen, bile, excrement, sweat, sputum, tears, spinal fluid, or stool. In some embodiments, the source of the sample are secretions. In some embodiments, the secretions are exosomes.

In some embodiments, the sample is a urine sample. In some embodiments, the urine sample is obtained after performing a digital rectal examination (DRE) of the subject. In some embodiments, the urine sample is obtained within 30 minutes after performing a DRE of the subject. In some embodiments, the urine sample is obtained from 30 minutes to 60 minutes after performing a DRE of the subject. In some embodiments, the urine sample is obtained from 30 minutes to 180 minutes after performing a DRE of the subject. In some embodiments, the urine sample is obtained within a day (e.g., within 24 hours) after performing a DRE of the subject. In some embodiments, the urine sample is obtained within 60 minutes after performing a DRE of the subject. In some embodiments, the urine sample is obtained within two hours after performing a DRE of the subject. In some embodiments, the urine sample is obtained within three hours, or about 180 minutes, after performing a DRE of the subject. In some embodiments, a urine sample is obtained from a subject who has not had a DRE. In some embodiments, the subject has not had a DRE within about 180 minutes before the subject's urine sample is obtained.

Without wishing to be bound by theory, it is believed that the DRE increases the urine sample's amount of expression of, e.g., concentration of the mRNA or protein expressed by, one or more of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. This increased concentration facilitates detection of the amount of gene expression, e.g., mRNA or protein expressed by, the one or more genes.

In some embodiments, a sample is combined with a buffer, e.g., for processing. In some embodiments, an amount of expression of each of the one or more genes described herein is determined from a composition, e.g., a solution or suspension, comprising the sample and a buffer. Buffers suitable for samples are known to those of skill in the art and can be determined based on the type of sample being collected. In some embodiments, the composition further comprises a preservative for adequate stability of the sample. In some embodiments, the buffer to sample ratio is 2:5. In some embodiments, the buffer to sample ratio is 1:5, 2:5, 3:5 or 4:5.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

In some embodiments, a subject is prostate biopsy-naïve. In some embodiments, a subject is prostate biopsy-prior negative. In some embodiments, a subject is prostate biopsy-prior negative for Grade Group ≥2 prostate cancer. In some embodiments, a subject has had a prior positive prostate biopsy result. In some embodiments, the prior positive prostate biopsy result is for GG1 prostate cancer. In some embodiments, one or more additional clinical variables are associated with the subject.

Assigning a Likelihood that a Prostate Biopsy of a Subject would Detect Grade Group ≥0.2 Prostate Cancer in the Subject

In some embodiments, the likelihood that a prostate biopsy of a subject would detect GG≥2 prostate cancer in the subject is presented as a risk score. In some embodiments, the risk score ranges from 0% (lowest risk) to 100% (highest risk). In some embodiments, the risk score ranges from 0.00 (lowest risk) to 1.00 (highest risk).

In some embodiments, the likelihood that a prostate biopsy of a subject would detect GG≥3 prostate cancer in the subject is presented as a risk score. In some embodiments, the risk score ranges from 0% (lowest risk) to 100% (highest risk). In some embodiments, the risk score ranges from 0.00 (lowest risk) to 1.00 (highest risk).

In some embodiments, the likelihood that a prostate biopsy of a subject would detect GG≥4 prostate cancer in the subject is presented as a risk score. In some embodiments, the risk score ranges from 0% (lowest risk) to 100% (highest risk). In some embodiments, the risk score ranges from 0.00 (lowest risk) to 1.00 (highest risk).

In some embodiments, the likelihood that a prostate biopsy of a subject would detect GG5 prostate cancer in the subject is presented as a risk score. In some embodiments, the risk score ranges from 0% (lowest risk) to 100% (highest risk). In some embodiments, the risk score ranges from 0.00 (lowest risk) to 1.00 (highest risk).

A computer-based analysis program can be used to translate the raw data generated by a detection assay (e.g., the presence, absence, or amount of a given marker or markers) into data of predictive value for a clinician, subject or subject's healthcare provider. The clinician, subject or subject's healthcare provider can access the raw data using any suitable means. The computer-based analysis program can provide the further benefit that the clinician, subject or subject's healthcare provider, who might not be trained in genetics or molecular biology, need not understand the raw data. The data can be presented directly to the clinician subject or subject's healthcare provider in its most useful form. This enables the clinician or healthcare provider to immediately utilize the information to optimize the care of the subject.

The information can be received, processed or transmitted to or from one or more laboratories conducting the assays, information providers, medical personnel, or subjects using any suitable method. For example, in some embodiments, a sample (e.g., a tissue sample, e.g., a biopsy, a whole blood, a plasma, a serum, a urine sample, a semen sample, a stool sample, a sputum sample, or a combination thereof) is obtained from a subject and submitted to a processing service (e.g., clinical lab at a medical facility, genomic profiling business, etc.), located in any part of the world (e.g., in a country different than the country where the subject resides or where the information is ultimately used) for processing, including to generate raw data. Where the sample comprises a tissue or other biological sample, the subject can visit a medical center to have the sample obtained and sent to the profiling center. Where the sample is a urine sample, the subject himself can collect the urine sample and send it to a processing center. Where the sample comprises previously determined biological information, the information can be directly sent to the processing service by the subject (e.g., an information card containing the information may be scanned by a computer and the data transmitted to a computer of the profiling center using an electronic communication systems). Once received by the processing service, the sample can be processed or analyzed (e.g., by a human, by a compute device, or a combination thereof).

The data can then be expressed in a format suitable for interpretation by the subject or a heath care provider of the subject, e.g., one or more medical personnel (e.g., a treating clinician, urologist, internist, physician assistant, nurse, or pharmacist). For example, rather than providing raw expression data, the expressed format can include a diagnosis or risk assessment (e.g., levels of the cancer markers described herein) for the subject, along with one or more recommendations for particular treatment options. The data can be output to the subject or subject's healthcare provider by any suitable means or method or displayed, e.g., visibly or audibly, to the medical personnel by any suitable method. In some embodiments, the data or their expressed format can be included in a report that can be printed, e.g., for the subject or a healthcare provider of the subject (e.g., at the point of care), or displayed on a computer monitor.

The raw data or additional information can be analyzed at the point of care or at a regional facility. The raw data can then be sent to a central processing facility for further analysis and/or to convert the raw data to information useful for the subject or healthcare provider of the subject. The central processing facility provides the advantage of privacy (all data is stored in a central facility with uniform security protocols), speed, and uniformity of data analysis. The central processing facility can then store or otherwise control the fate of the data. For example, using an electronic communication system, the central facility can provide data to the subject or healthcare provider of the subject, e.g., via a compute device of the subject or a compute device of the healthcare provider of the subject.

In some embodiments, the subject or the subject's healthcare provider is able to directly access the data using the electronic communication system. The subject may choose (e.g., via their compute device) further intervention or counseling based on the results.

In some embodiments, the data (e.g., to be sent to the central processing facility and/or received at the central processing facility) is used for research use. For example, the data may be used to further optimize the inclusion or elimination of markers as useful indicators of a particular condition or stage of disease or as a companion diagnostic to determine a treatment course of action.

The present methods comprise obtaining a risk score comprising performing the equation

$$\frac{{e}^{calibrated logit value}}{1 + {e}^{calibrated logit value}}$$

wherein the risk score indicates the likelihood that a prostate biopsy of the subject would detect GG≥2 prostate cancer in the subject. In some embodiments, the risk score indicates the likelihood that a prostate biopsy of the subject would detect Grade Group ≥3 prostate cancer. In some embodiments, the risk score indicates the likelihood that a prostate biopsy of the subject would detect Grade Group ≥4 prostate cancer. In some embodiments, the risk score indicates the likelihood that a prostate biopsy of the subject would detect Grade Group 5 prostate cancer.

As used herein, “Crt” refers to the cycle threshold value identified by a method described herein for detecting the amount of expression of a gene, and “e” is Euler's number.

Risk scores are calculated using a logistic regression algorithm generated using an elastic-net regularization path. Generation of regularization paths is described, for example and without limitation, in Friedman et a., 2010, Journal of Statistical Software, 33(1), 1-22 and Tay et a., 2023, Journal of Statistical Software, 106(1), 1-31. The algorithm included the 17 most informative genes, including 13 from the discovery analysis: four GG≥2-specific (APOC1, B3GNT6, NKAIN1, and SCHLAP1); nine cancer-specific (PCGEM1, SPON2, TRGV9, PCA3, OR51E2, CAMKK2, TFF3, PCAT14, and TMSB15A); four curated markers (HOXC6, ERG, TMPRSS2:ERG, and KLK4); and the reference gene KLK3. Two additional algorithms (i.e., “biomarker+clinical factor” and “biomarker+clinical factor+prostate volume”) were generated to include clinical variables, which progressively improve algorithm performance. Algorithms were calibrated to account for differences in outcome prevalence between cohorts. Illustrative, non-limiting methods for algorithm calibration are described in Vergouwe et a, 2017, Stat Med. 36(28):4529-4539 and Moons et al., 2015, Ann Intern Med. 2015 Jan. 6; 162(1):W1-73.

In some embodiments, one or more gene algorithm coefficients or algorithm intercept values provided herein are those of Table B, Table C, Table D or Table E. In some embodiments, one or more gene algorithm coefficients provided in Table C or Table E vary by ±10%, ±5%, ±2% or ±1%. Unless expressly stated otherwise, the one or more gene algorithm coefficients of Table C or Table E do not vary. Although the gene algorithm coefficients of Table B and Table C were trained on prostate biopsy-naïve and prostate biopsy-prior negative patients, the gene algorithm coefficients are useful in subjects other than prostate biopsy-naïve and prostate biopsy-prior negative patients. Although the gene algorithm coefficients of Table D and Table E were trained on only prostate biopsy-naïve patients, the gene algorithm coefficients of Table D and Table E me useful in subjects other than prostate biopsy-naïve patients.

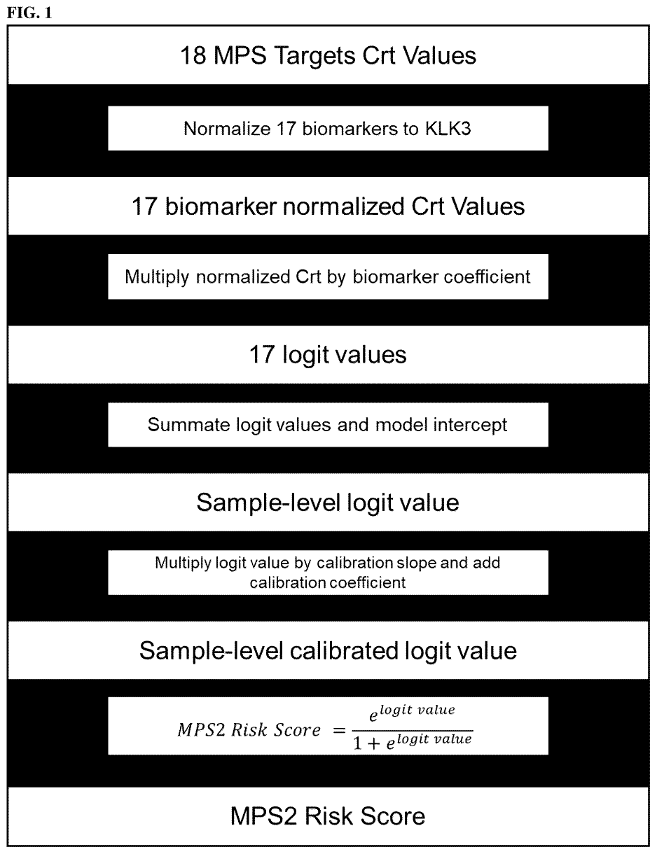

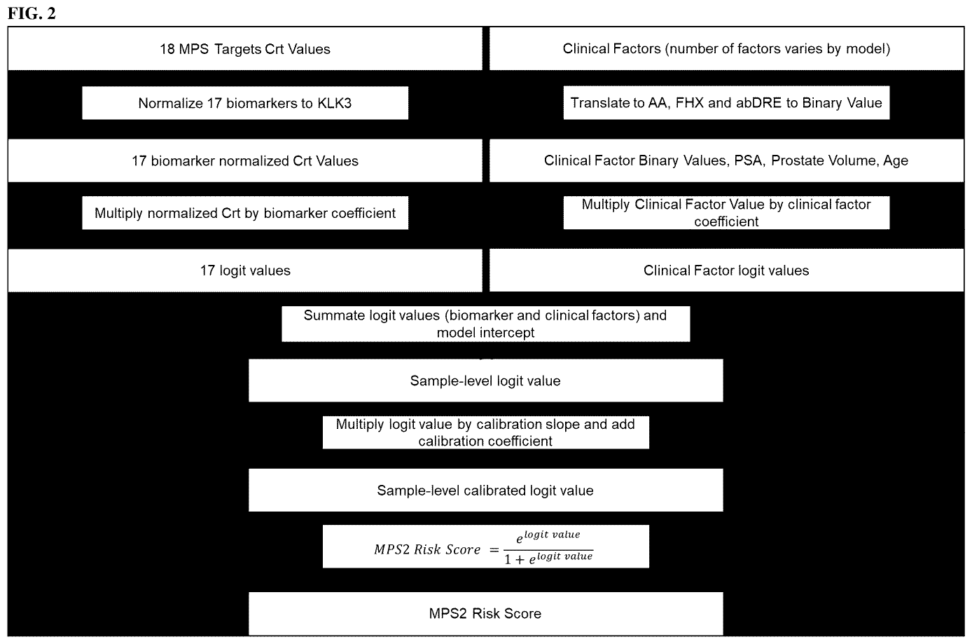

The Risk score calculation for biomarker only algorithms and biomarker+clinical factor-containing algorithms is illustrated in five steps (see FIG. 1 and FIG. 2). This process (e.g., implementable or implemented by a compute device, such as compute device 801 of FIG. 13) is summarized below:
1. The qPCR-based protocol results in Target Gene cycle threshold values (Crt), which are used as raw data input into the algorithm.
2. Crt values for the 17 informative genes are normalized to the Crt value of KLK3 using the following equation:

$$Target Gene Normalized Crt = Target Gene Crt - KLK 3 Crt$$

a. If a clinical factor-containing algorithm is being utilized, for binary clinical factors (yes/no), the status is converted to a binary value (I/O, respectively). The binary clinical factors include African Ancestry, Family History of Prostate Cancer, Abnormal DRE (e.g., a hard mass or nodule, induration, or asymmetry), and prior negative prostate biopsy result.
3. Each Target Gene Normalized Crt is multiplied by their respective algorithm coefficient (Table C and Table E) to generate log it values.
   a. If a clinical factor-containing algorithm is being utilized, each clinical factor value is also multiplied by their respective algorithm coefficient to generate log it values. If all available clinical factors are included, and the subject's prostate volume is unavailable, algorithm coefficients are as set forth in the Biomarker+Clinical Factor (without prostate volume) column of Table C or Table E. If all available clinical factors included, and the subject's prostate volume is available, algorithm coefficients are as set forth in the Biomarker+Clinical Factor+Prostate Volume Coefficient column of Table C or Table E.
      i. If a subject's age is being used, the age is multiplied by the corresponding algorithm coefficient to provide a log it value.
      ii. If a subject's PSA concentration (e.g., in ng/mL) is being used, the age is multiplied by the corresponding algorithm coefficient to provide a log it value.
      iii. If African Ancestry, Family History of Prostate Cancer, Abnormal DRE, or prior negative prostate biopsy result are being used, the binary value (1 being yes, 0 being no) is multiplied by the corresponding algorithm coefficient to provide a log it value.
      iv. If the subject's prostate volume (e.g., in cm³) is being used, the prostate volume is multiplied by the corresponding algorithm coefficient to provide a log it value.
4. Log it values are summated along with the algorithm intercept value (Table C and Table E) producing the sample-level log it value.
5. The sample-level log it value is multiplied by the calibration slope value followed by addition of the calibration intercept value to produce the sample-level calibrated log it value (Table G).
6. The sample-level calibrated log it value is input in the logistic regression equation below to calculate the Risk score:

$$Risk Score \frac{{e}^{calibrated logit value}}{1 + {e}^{calibrated logit value}}$$

TABLE B
Gene algorithm coefficients. Algorithm trained on prostate
biopsy-naïve and prostate biopsy-prior negative patients.
The biomarker-only algorithm intercept value is 6.80 ± 0.15,
the biomarker + clinical factor algorithm intercept value is 5.90 ± 0.84,
and the biomarker + clinical factor + prostate volume
algorithm intercept value is 6.68 ± 0.63.

| | | Biomarker + | Biomarker + |
|---|---|---|---|
| Gene or | | Clinical Factor | Clinical Factor + |
| Clinical | Biomarker-only | (without prostate | Prostate Volume |
| Factor | Coefficient | volume) Coefficient | Coefficient |
| T2ERG | 0.14 ± 0.15 | 0.11 ± 0.84 | 0.15 ± 0.63 |
| SCHLAP1 | 0.22 ± 0.15 | 0.17 ± 0.84 | 0.21 ± 0.63 |
| OR51E2 | 0.25 ± 0.15 | 0.20 ± 0.84 | 0.23 ± 0.63 |
| APOC1 | −0.06 ± 0.15 | −0.08 ± 0.84 | −0.09 ± 0.63 |
| PCAT14 | 0.13 ± 0.15 | 0.14 ± 0.84 | 0.16 ± 0.63 |
| CAMKK2 | −0.35 ± 0.15 | −0.26 ± 0.84 | −0.28 ± 0.63 |
| PCA3.1 | 0.13 ± 0.15 | 0.08 ± 0.84 | 0.07 ± 0.63 |
| NKAIN1 | −0.08 ± 0.15 | −0.07 ± 0.84 | −0.09 ± 0.63 |
| B3GNT6 | 0.05 ± 0.15 | 0.05 ± 0.84 | 0.07 ± 0.63 |
| TFF3 | 0.17 ± 0.15 | 0.19 ± 0.84 | 0.21 ± 0.63 |
| SPON2 | 0.11 ± 0.15 | 0.16 ± 0.84 | 0.17 ± 0.63 |
| PCGEM1 | −0.14 ± 0.15 | −0.17 ± 0.84 | −0.15 ± 0.63 |
| TRGV9 | 0.13 ± 0.15 | 0.10 ± 0.84 | 0.18 ± 0.63 |
| TMSB15A | 0.17 ± 0.15 | 0.15 ± 0.84 | 0.21 ± 0.63 |
| ERG | 0.02 ± 0.15 | 0.02 ± 0.84 | 0.03 ± 0.63 |
| KLK4 | 0.00 ± 0.15 | 0.15 ± 0.84 | 0.21 ± 0.63 |
| HOXC6 | 0.01 ± 0.15 | 0.06 ± 0.84 | 0.00 ± 0.63 |
| Age | | 0.00 ± 0.84 | 0.02 ± 0.63 |
| African | | 0.83 ± 0.84 | 1.23 ± 0.63 |
| Ancestry | | | |
| Family | | 0.15 ± 0.84 | 0.29 ± 0.63 |
| History | | | |
| Abnormal | | 0.89 ± 0.84 | 1.09 ± 0.63 |
| DRE | | | |
| Prostate | | −0.85 ± 0.84 | −0.62 ± 0.63 |
| Biopsy-Prior | | | |
| Negative | | | |
| PSA | | 0.07 ± 0.84 | 0.09 ± 0.63 |
| Prostate | | | −0.02 ± 0.63 |
| Volume | | | |

TABLE C
Gene algorithm coefficients. Algorithm trained on prostate
biopsy-naïve and prostate biopsy-prior negative patients.
The biomarker-only algorithm intercept value is 6.80,
the biomarker + clinical factor algorithm intercept value is 5.90,
and the biomarker + clinical factor + prostate
volume algorithm intercept value is 6.68.

| | | Biomarker + | Biomarker + |
|---|---|---|---|
| Gene or | | Clinical Factor | Clinical Factor + |
| Clinical | Biomarker only- | (without prostate | Prostate Volume |
| Factor | Coefficient | volume) Coefficient | Coefficient |
| T2ERG | 0.14 | 0.11 | 0.15 |
| SCHLAP1 | 0.22 | 0.17 | 0.21 |
| OR51E2 | 0.25 | 0.20 | 0.23 |
| APOC1 | −0.06 | −0.08 | −0.09 |
| PCAT14 | 0.13 | 0.14 | 0.16 |
| CAMKK2 | −0.35 | −0.26 | −0.28 |
| PCA3.1 | 0.13 | 0.08 | 0.07 |
| NKAIN1 | −0.08 | −0.07 | −0.09 |
| B3GNT6 | 0.05 | 0.05 | 0.07 |
| TFF3 | 0.17 | 0.19 | 0.21 |
| SPON2 | 0.11 | 0.16 | 0.17 |
| PCGEM1 | −0.14 | −0.17 | −0.15 |
| TRGV9 | 0.13 | 0.10 | 0.18 |
| TMSB15A | 0.17 | 0.15 | 0.21 |
| ERG | 0.02 | 0.02 | 0.03 |
| KLK4 | 0.00 | 0.15 | 0.21 |
| HOXC6 | 0.01 | 0.06 | 0.00 |
| Age | | 0.00 | 0.02 |
| African | | 0.83 | 1.23 |
| Ancestry | | | |
| Family | | 0.15 | 0.29 |
| History | | | |
| Abnormal | | 0.89 | 1.09 |
| DRE | | | |
| Prostate | | −0.85 | −0.62 |
| Biopsy-Prior | | | |
| Negative | | | |
| PSA | | 0.07 | 0.09 |
| Prostate | | | −0.02 |
| Volume | | | |

TABLE D
Gene algorithm coefficients. Algorithm trained on only prostate
biopsy-naïve patients. The biomarker-only algorithm
intercept value is 8.39 ± 0.15, the biomarker + clinical factor algorithm
intercept value is 7.52 ± 0.84, and the biomarker + clinical
factor + prostate volume algorithm value is 6.25 ± 0.63.

| | | Biomarker + | Biomarker + |
|---|---|---|---|
| Gene or | | Clinical Factor | Clinical Factor + |
| Clinical | Biomarker only- | (without prostate | Prostate Volume |
| Factor | Coefficient | volume) Coefficient | Coefficient |
| T2ERG | 0.18 ± 0.15 | 0.15 ± 0.84 | 0.14 ± 0.63 |
| SCHLAP1 | 0.19 ± 0.15 | 0.16 ± 0.84 | 0.13 ± 0.63 |
| OR51E2 | 0.30 ± 0.15 | 0.23 ± 0.84 | 0.15 ± 0.63 |
| APOC1 | −0.07 ± 0.15 | −0.08 ± 0.84 | −0.07 ± 0.63 |
| PCAT14 | 0.14 ± 0.15 | 0.14 ± 0.84 | 0.13 ± 0.63 |
| CAMKK2 | −0.40 ± 0.15 | −0.24 ± 0.84 | −0.25 ± 0.63 |
| PCA3.1 | 0.10 ± 0.15 | 0.09 ± 0.84 | 0.09 ± 0.63 |
| NKAIN1 | −0.12 ± 0.15 | −0.10 ± 0.84 | −0.09 ± 0.63 |
| B3GNT6 | 0.08 ± 0.15 | 0.04 ± 0.84 | 0.06 ± 0.63 |
| TFF3 | 0.17 ± 0.15 | 0.16 ± 0.84 | 0.11 ± 0.63 |
| SPON2 | 0.27 ± 0.15 | 0.17 ± 0.84 | 0.14± 0.63 |
| PCGEM1 | −0.27 ± 0.15 | −0.11 ± 0.84 | −0.11 ± 0.63 |
| TRGV9 | 0.20 ± 0.15 | 0.12 ± 0.84 | 0.15 ± 0.63 |
| TMSB15A | 0.28 ± 0.15 | 0.21 ± 0.84 | 0.20 ± 0.63 |
| ERG | 0.02 ± 0.15 | 0.03 ± 0.84 | 0.02 ± 0.63 |
| KLK4 | 0.12± 0.15 | 0.10 ± 0.84 | 0.13 ± 0.63 |
| HOXC6 | 0.08 ± 0.15 | 0.10 ± 0.84 | 0.04 ± 0.63 |
| Age | | 0.00 ± 0.84 | 0.00 ± 0.63 |
| African | | 0.66 ± 0.84 | 0.59 ± 0.63 |
| Ancestry | | | |
| Family | | 0.05 ± 0.84 | 0.10 ± 0.63 |
| History | | | |
| Abnormal | | 0.98 ± 0.84 | 1.06 ± 0.63 |
| DRE | | | |
| PSA | | 0.00 ± 0.84 | 0.10 ± 0.63 |
| Prostate | | | −0.02 ± 0.63 |
| Volume | | | |

TABLE E
Gene algorithm coefficients. Algorithm trained on only prostate
biopsy-naïve patients. The biomarker-only algorithm
intercept value is 8.39, the biomarker + clinical factor
algorithm intercept value is 7.52, and the biomarker + clinical
factor + prostate volume algorithm intercept value is 6.25.

| | | Biomarker + | Biomarker + |
|---|---|---|---|
| Gene or | | Clinical Factor | Clinical Factor + |
| Clinical | Biomarker only- | (without prostate | Prostate Volume |
| Factor | Coefficient | volume) Coefficient | Coefficient |
| T2ERG | 0.18 | 0.15 | 0.14 |
| SCHLAP1 | 0.19 | 0.16 | 0.13 |
| OR51E2 | 0.30 | 0.23 | 0.15 |
| APOC1 | −0.07 | −0.08 | −0.07 |
| PCAT14 | 0.14 | 0.14 | 0.13 |
| CAMKK2 | −0.40 | −0.24 | −0.25 |
| PCA3.1 | 0.10 | 0.09 | 0.09 |
| NKAIN1 | −0.12 | −0.10 | −0.09 |
| B3GNT6 | 0.08 | 0.04 | 0.06 |
| TFF3 | 0.17 | 0.16 | 0.11 |
| SPON2 | 0.27 | 0.17 | 0.14 |
| PCGEM1 | −0.27 | −0.11 | −0.11 |
| TRGV9 | 0.20 | 0.12 | 0.15 |
| TMSB15A | 0.28 | 0.21 | 0.20 |
| ERG | 0.02 | 0.03 | 0.02 |
| KLK4 | 0.12 | 0.10 | 0.13 |
| HOXC6 | 0.08 | 0.10 | 0.04 |
| Age | | 0.00 | 0.00 |
| African | | 0.66 | 0.59 |
| Ancestry | | | |
| Family | | 0.05 | 0.10 |
| History | | | |
| Abnormal | | 0.98 | 1.06 |
| DRE | | | |
| PSA | | 0.00 | 0.10 |
| Prostate | | | −0.02 |
| Volume | | | |

TABLE F
Algorithm calibration intercept and calibration slope values

| | | Calibration | Calibration |
|---|---|---|---|
| | | Intercept | Slope |
| Algorithm | Algorithm Type | Value | Value |
| Algorithm trained | Biomarkers Only | −1.41 +/− 0.38 | 1.13 +/− 0.07 |
| on prostate | Biomarkers + | −1.45 +/− 0.44 | 1.30 +/− 0.07 |
| biopsy- naïve and | Clinical Factors | | |
| prostate biopsy- | Biomarkers + | −1.41 +/− 0.38 | 1.08 +/− 0.26 |
| prior negative | Clinical Factors + | | |
| patients (Table B | Prostate Volume | | |
| or Table C) | | | |
| Algorithm trained | Biomarkers Only | −1.02 +/− 0.38 | 1.05 +/− 0.07 |
| on only prostate | Biomarkers + | −1.00 +/− 0.44 | 1.22 +/− 0.07 |
| biopsy- naïve | Clinical Factors | | |
| patients (Table D | Biomarkers + | −1.02 +/− 0.38 | 1.35 +/− 0.26 |
| or Table E) | Clinical Factors + | | |
| | Prostate Volume | | |

TABLE G
Algorithm calibration intercept and calibration slope values

| | | Calibration | Calibration |
|---|---|---|---|
| | Algorithm | Intercept | Slope |
| Algorithm | Type | Value | Value |
| Algorithm | Biomarkers Only | −1.41 | 1.13 |
| trained on | Biomarkers + | −1.45 | 1.30 |
| prostate | Clinical Factors | | |
| biopsy | Biomarkers + | −1.41 | 1.08 |
| naïve and | Clinical Factors + | | |
| prostate | Prostate Volume | | |
| biopsy | | | |
| prior | | | |
| negative | | | |
| patients | | | |
| (Table B | | | |
| or Table C) | | | |
| Algorithm | Biomarkers Only | −1.02 | 1.05 |
| trained on | Biomarkers + | −1.00 | 1.22 |
| only | Clinical Factors | | |
| prostate | Biomarkers + | −1.02 | 1.35 |
| biopsy | Clinical Factors + | | |
| naïve | Prostate Volume | | |
| (Table D | | | |
| or Table E) | | | |

The present methods comprise normalizing the amount of expression of each of the one or more genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 to an amount of expression of a reference gene to provide a normalized target gene value for each of the genes. In some embodiments, the reference gene is KLK3, CYPB561A3, EEF1A2, GAPDH, HPN, KLK2, KLK4, LBH, NUDT8, SPDEF, or TRGV. In some embodiments, the reference gene is KLK3.

The present methods further comprise multiplying each normalized target gene value by a corresponding gene algorithm coefficient to provide a log it value for each of the genes (a “gene log it value”). In some embodiments, the corresponding gene algorithm coefficient is a corresponding gene algorithm coefficient set forth in Table B, e.g., a gene algorithm coefficient set forth in Table C. In some embodiments, the corresponding gene algorithm coefficient is a corresponding gene algorithm coefficient set forth in Table D, e.g., a gene algorithm coefficient set forth in Table E. The skilled person will readily appreciate that the useful gene algorithm coefficient can depend on whether the subject has biomarker information available only (i.e., only an amount of expression of each of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6), biomarker information and any one or more clinical factor values (i.e., of age, African ancestry, family history of prostate cancer, an abnormal DRE, PSA levels, or a combination thereof), or biomarker information, any one or more clinical factors, and prostate volume. As a non-limiting example, where the normalized target gene is TMPRSS2-ERG (also known as 72ERG), and the subject has biomarker information only, the corresponding gene algorithm coefficient set forth in Table C is 0.14. In another non-limiting example, where the normalized target gene is TFF3, the subject has biomarker information, clinical factor, and prostate volume information available, and the subject is prostate biopsy-naïve, the corresponding gene algorithm coefficient set forth in Table E is 0.11.

In some embodiments, the present methods utilize one or more clinical factor values (i.e., of age, African ancestry, family history of prostate cancer, an abnormal DRE, PSA levels, prostate volume, or a combination thereof) and further comprise multiplying each clinical factor value by a corresponding clinical factor coefficient to provide a log it value for each of the clinical factor values.

The present methods further comprise summing 1) the log it values and 2) an algorithm intercept value to provide a sample log it value. In some embodiments, the algorithm coefficients are set forth in Table C, and the algorithm intercept value for the biomarker information only is 6.80, for the biomarker information and any one or more clinical factors is 5.90, and for the biomarker information, any one or more clinical factors, and prostate volume is 6.68. In some embodiments, the algorithm coefficients are set forth in Table E, and the algorithm intercept value for the biomarker information only is 8.39, for the biomarker information and any one or more clinical factors is 7.52, and for the biomarker information, any one or more clinical factors, and prostate volume is 6.25.

The present methods further comprise multiplying the sample log it value by a calibration slope value to provide a product, and adding to the product a calibration intercept value to provide a calibrated log it value. In some embodiments, the calibration slope value and calibration intercept value are set forth in Table F or Table G. Although the calibration slope values and calibration intercept values of Table F and Table G were trained on prostate biopsy-naïve and prostate biopsy-prior negative patients, or on only prostate biopsy-naïve patients, the calibration slope values and calibration intercept values of Table F and Table G are useful in subjects other than prostate biopsy-naïve and prostate biopsy-prior negative patients, and other than only prostate biopsy-naïve patients.

The present methods further comprise obtaining a risk score indicating the likelihood that a prostate biopsy of a subject would detect GG≥2 prostate cancer in the subject, wherein obtaining the risk score comprises performing the equation

$$\frac{{e}^{calibrated logit value}}{1 + {e}^{calibrated logit value}}$$

In some embodiments, the methods disclosed herein comprise transmitting the data/information (e.g., between compute devices). For example, data/information derived from the detection and/or quantification of the target may be transmitted to another device and/or instrument. In some instances, the information obtained from an algorithm may also be transmitted to another device and/or instrument. Transmission of the data/information may comprise the transfer of data/information from a first source to a second source. The first and second sources may be in the same approximate location (e.g., within the same room, building, block, campus). Alternatively, first and second sources may be in multiple locations (e.g., multiple cities, states, countries, continents, etc.).

Transmission of the data/information can comprise digital transmission or analog transmission. Digital transmission may comprise the physical transfer of data (a digital bit stream) over a point-to-point or point-to-multipoint communication channel. Examples of such channels are copper wires, optical fibers, wireless communication channels, and storage media. The data may be represented as an electromagnetic signal, such as an electrical voltage, radiowave, microwave, or infrared signal.

Analog transmission may comprise the transfer of a continuously varying analog signal. The messages can either be represented by a sequence of pulses by means of a line code (baseband transmission), or by a limited set of continuously varying wave forms (passband transmission), using a digital modulation method. The passband modulation and corresponding demodulation (also known as detection) can be carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion.

In some embodiments, the present methods further comprise outputting, e.g., to the subject or the subject's healthcare provider, data relating to an amount of expression of one or more genes disclosed herein, a risk score, a risk threshold value or a risk category. In some embodiments, the methods further comprise outputting, e.g., to the subject or the subject's healthcare provider, additional information, e.g., a recommendation that the subject undergo a prostrate biopsy, a recommendation that the subject not undergo a prostate biopsy, a statement that a prostate biopsy of the subject is unnecessary or a recommendation for a therapeutic intervention or treatment option. In some embodiments, the present methods further comprise generating (e.g., via a compute device, such as compute device 801 of FIG. 13) a report comprising a risk score, risk threshold value or a risk category (e.g., low risk or increasing risk). In some embodiments, the report comprises a risk score. The risk score indicates a likelihood that GG≥2 prostate cancer would be detected from a prostate biopsy of the subject. In some embodiments, the report comprises a prostate cancer risk category (e.g., low risk or increasing risk). In some embodiments, the present methods further comprise informing the subject or a healthcare provider of the subject of the risk score, risk threshold value, or risk category. In some embodiments, the methods further comprise generating a report comprising a risk score, risk threshold value or a prostate cancer risk category (i.e., low risk or increasing risk). In some embodiments, the report comprises a risk score. The risk score indicates a likelihood that GG≥2 prostate cancer would be detected from a prostate biopsy of the subject. In some embodiments, the report comprises a prostate cancer risk category (i.e., low risk or increasing risk). In some embodiments, the report is accessible by or provided to the subject or the subject's healthcare provider. In some embodiments, the report is accessible or provided as a digital or paper copy. In some embodiments, the report is delivered to the subject or subject's healthcare provider by a digital format as described herein (e.g., via electronic mail), or via a courier if the report is in paper copy.

In some embodiments, the subject's healthcare provider does not recommend that the subject undergo a prostate biopsy where the subject's risk category is low-risk. In some embodiments, where the subject's risk category is low-risk (ii) the subject does not undergo a prostate biopsy or (ii) the subject's healthcare provider does not recommend that the subject undergo a prostate biopsy.

In some embodiments, the subject's healthcare provider recommends that the subject undergo a prostate biopsy where the subject's risk category is increasing-risk. In some embodiments, where the subject's risk category is increasing-risk (ii) the subject undergoes a prostate biopsy or (ii) the subject's healthcare provider recommends that the subject undergo a prostate biopsy.

In some embodiments, the report comprises recommendation for a prostate biopsy. In some embodiments, the report comprises a treatment option. In some embodiments, the report comprises a treatment option for GG≥2 prostate cancer.

### Algorithms and Algorithm Coefficients

The algorithms and algorithm coefficients provided herein are novel, nonobvious and unconventional; enable assignment of a likelihood that a prostate biopsy of a subject would detect GG≥2 prostate cancer in the subject; and allow those assigned as having a low risk of having GG≥22 prostate cancer avoid a biopsy or unnecessary treatment and, accordingly, its associated side effects. The algorithms and algorithm coefficients are useful in the methods as provided herein and are useful to reduce the number of avoidable prostate biopsies, sparing healthy subjects or subjects having GG<2 prostate cancer from a costly, invasive procedure.

### Algorithm Performance

Algorithm performance for the methods described herein can be determined by analyzing the Area Under the Curve (AUC) derived from Receiver Operator Characteristic (ROC) curves. ROC curves are graphical plots that illustrate the ability of a binary classifier system as its discrimination threshold is varied. ROC curves are plotted with specificity against the sensitivity, with sensitivity on the y-axis and 1-sensitivity on the x-axis. “Sensitivity” is calculated by dividing the number of true positives by the sum of true positives and false negatives. The “specificity” is calculated by dividing the number of true negatives by the sum of true negatives and false positives. In some embodiments, ROC curves are generated based on individual amounts of expression of each gene. In some embodiments, ROC curves are generated based on a combination of amounts of expression of each gene.

In some embodiments, the AUC value of the methods described herein is greater than 0.50. In some embodiments, the AUC value of the methods described herein is at least 0.60. In some embodiments, the AUC value of the methods described herein is at least 0.70. In some embodiments, the AUC value of the methods described herein is at least 0.71. In some embodiments, the AUC value of the methods described herein is at least 0.72. In some embodiments, the AUC value of the methods described herein is at least 0.73. In some embodiments, the AUC value the methods described herein is at least 0.74. In some embodiments, the AUC value of the methods described herein is at least 0.75. In some embodiments, the AUC value of the methods described herein is at least 0.76. In some embodiments, the AUC value of the methods described herein is at least 0.77. In some embodiments, the AUC value of the methods described herein is at least 0.78. In some embodiments, the AUC value of the methods described herein is at least 0.79. In some embodiments, the AUC value of the methods described herein is at least 0.80. In some embodiments, the AUC value of the methods described herein is at least 0.81. In some embodiments, the AUC value of the methods described herein is at least 0.82. In some embodiments, the AUC value of the methods described herein is at least 0.83. In some embodiments, the AUC value of the methods described herein is at least 0.84. In some embodiments, the AUC value of the methods described herein is at least 0.85. In some embodiments, the AUC value of the methods described herein is at least 0.86. In some embodiments, the AUC value of the methods described herein is at least 0.87. In some embodiments, the AUC value of the methods described herein is at least 0.88. In some embodiments, the AUC value of the methods described herein is at least 0.89. In some embodiments, the AUC value of the methods described herein is at least 0.90.

In some embodiments, the specificity of the methods described herein is greater than 0.20. In some embodiments, the specificity of the methods described herein is greater than 0.30. In some embodiments, the specificity of the methods described herein is greater than 0.40. In some embodiments, the specificity of the methods described herein is greater than 0.50. In some embodiments, the specificity of the methods described herein is at least 0.60. In some embodiments, the specificity of the methods described herein is at least 0.70. In some embodiments, the specificity of the methods described herein is at least 0.71. In some embodiments, the specificity of the methods described herein is at least 0.72. In some embodiments, the specificity of the methods described herein is at least 0.73. In some embodiments, the specificity the methods described herein is at least 0.74. In some embodiments, the specificity of the methods described herein is at least 0.75. In some embodiments, the specificity of the methods described herein is at least 0.76. In some embodiments, the specificity of the methods described herein is at least 0.77. In some embodiments, the specificity of the methods described herein is at least 0.78. In some embodiments, the specificity of the methods described herein is at least 0.79. In some embodiments, the specificity of the methods described herein is at least 0.80. In some embodiments, the specificity of the methods described herein is at least 0.81. In some embodiments, the specificity of the methods described herein is at least 0.82. In some embodiments, the specificity of the methods described herein is at least 0.83. In some embodiments, the specificity of the methods described herein is at least 0.84. In some embodiments, the specificity of the methods described herein is at least 0.85. In some embodiments, the specificity of the methods described herein is at least 0.86. In some embodiments, the specificity of the methods described herein is at least 0.87. In some embodiments, the specificity of the methods described herein is at least 0.88. In some embodiments, the specificity of the methods described herein is at least 0.89. In some embodiments, the specificity of the methods described herein is at least 0.90. In some embodiments, the specificity of the methods described herein is at least 0.91. In some embodiments, the specificity of the methods described herein is at least 0.92. In some embodiments, the specificity of the methods described herein is at least 0.93. In some embodiments, the specificity of the methods described herein is at least 0.94. In some embodiments, the specificity of the methods described herein is at least 0.95. In some embodiments, the specificity of the methods described herein is at least 0.96. In some embodiments, the specificity of the methods described herein is at least 0.97. In some embodiments, the specificity of the methods described herein is at least 0.98. In some embodiments, the specificity of the methods described herein is at least 0.99.

In some embodiments, the sensitivity of the methods described herein is greater than 0.50. In some embodiments, the sensitivity of the methods described herein is at least 0.60. In some embodiments, the sensitivity of the methods described herein is at least 0.70. In some embodiments, the sensitivity of the methods described herein is at least 0.71. In some embodiments, the sensitivity of the methods described herein is at least 0.72. In some embodiments, the sensitivity of the methods described herein is at least 0.73. In some embodiments, the sensitivity the methods described herein is at least 0.74. In some embodiments, the sensitivity of the methods described herein is at least 0.75. In some embodiments, the sensitivity of the methods described herein is at least 0.76. In some embodiments, the sensitivity of the methods described herein is at least 0.77. In some embodiments, the sensitivity of the methods described herein is at least 0.78. In some embodiments, the sensitivity of the methods described herein is at least 0.79. In some embodiments, the sensitivity of the methods described herein is at least 0.80. In some embodiments, the sensitivity of the methods described herein is at least 0.81. In some embodiments, the sensitivity of the methods described herein is at least 0.82. In some embodiments, the sensitivity of the methods described herein is at least 0.83. In some embodiments, the sensitivity of the methods described herein is at least 0.84. In some embodiments, the sensitivity of the methods described herein is at least 0.85. In some embodiments, the sensitivity of the methods described herein is at least 0.86. In some embodiments, the sensitivity of the methods described herein is at least 0.87. In some embodiments, the sensitivity of the methods described herein is at least 0.88. In some embodiments, the sensitivity of the methods described herein is at least 0.89. In some embodiments, the sensitivity of the methods described herein is at least 0.90. In some embodiments, the sensitivity of the methods described herein is at least 0.91. In some embodiments, the sensitivity of the methods described herein is at least 0.92. In some embodiments, the sensitivity of the methods described herein is at least 0.93. In some embodiments, the sensitivity of the methods described herein is at least 0.94. In some embodiments, the sensitivity of the methods described herein is at least 0.95. In some embodiments, the sensitivity of the methods described herein is at least 0.96. In some embodiments, the sensitivity of the methods described herein is at least 0.97. In some embodiments, the sensitivity of the methods described herein is at least 0.98. In some embodiments, the sensitivity of the methods described herein is at least 0.99.

The risk threshold value is a cut-off analysis that takes into account the sensitivity, specificity, negative predictive value (NPV) and positive predictive value (PPV) useful for clinical utility. In some embodiments of the present methods, a risk threshold value is predetermined. Risk scores that are equal to or below the risk threshold value are deemed as being “low risk” for GG≥2 prostate cancer. Risk scores that are above the risk threshold value are deemed as being “increasing risk” for GG≥2 prostate cancer. A risk threshold value may be determined, for example and without limitation, based on accepted standards in healthcare or in the healthcare market, including governmental regulations.

In some embodiments, the method further comprises comparing the risk score to a predetermined risk threshold value, and assigning a risk category based on whether the risk score is a) less than or equal to the risk threshold value, or b) higher than the threshold value. In some embodiments, the risk category is a low-risk category where the risk score is a) less than or equal to the risk threshold value, and an increasing-risk category where the risk score is b) higher than the risk threshold value.

In some embodiments, a risk threshold value is about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, or more than 0.20. In some embodiments, a risk threshold is between 0.04 and 0.20, or any range therebetween.

Results of amounts of expression may be analyzed in any of a variety of ways. In some embodiments, the results are analyzed using a univariate, or single-variable analysis (SV). In some embodiments, the results are analyzed using multivariate analysis (MV).

The generation of ROC curves and analysis of a population of samples can be used to establish the risk threshold used to distinguish between different subject sub-groups. For example, the risk threshold can be used to distinguish between a high likelihood of detecting Grade Group ≥2 prostate cancer from a subject's prostate biopsy and a low likelihood of detecting Grade Group ≥2 prostate cancer from a subject's prostate biopsy. The risk threshold can be used to distinguish between a high likelihood of detecting Grade Group ≥3 prostate cancer from a subject's prostate biopsy and a low likelihood of detecting Grade Group ≥3 prostate cancer from a subject's prostate biopsy. The risk threshold can be used to distinguish between a high likelihood of detecting Grade Group 4 prostate cancer from a subject's prostate biopsy and a low likelihood of detecting Grade Group 4 prostate cancer from a subject's prostate biopsy. In some embodiments, the risk threshold can distinguish between subjects at low risk and subjects at increasing risk of Grade Group ≥2 prostate cancer (e.g., GG2, GG3, GG4, or GG5). In some embodiments, the risk threshold may distinguish between subjects with a non-aggressive cancer and an aggressive cancer.

In some embodiments, each referenced specificity and/or sensitivity is achievable where the urine sample is obtained within one hour after a subject's digital rectal examination (DRE). In some embodiments, each referenced specificity and/or sensitivity is achievable where the urine sample is obtained from 30 minutes to 60 minutes after a subject's DRE. In some embodiments, the urine sample is obtained from 30 minutes to 180 minutes after a subject's DRE. In some embodiments, the urine sample is obtained within one hour after a subject's DRE. In some embodiments, the urine sample is obtained within two hours after a subject's DRE. In some embodiments, the urine sample is obtained within three hours after a subject's DRE. In some embodiments, the urine sample is obtained on the same day (e.g., within 24 hours) of a subject's DRE.

### II. Prognosis, Diagnosis or Treatment

After assigning a likelihood that a prostate biopsy of a subject would detect Grade Group (GG)≥2 prostate cancer in the subject, a report may be forwarded to a healthcare provider of the subject (e.g., from a first compute device to a second compute device). In some embodiments, the healthcare provider does not recommend a prostate biopsy where the subject's risk category is low-risk. In some embodiments, where the subject's risk category is increasing-risk, the subject undergoes a prostate biopsy or the subject's healthcare provider recommends that the subject undergo a prostate biopsy. In some embodiments, the healthcare provider recommends a prostate biopsy where the subject's risk category is increasing-risk. The healthcare provider may then provide a prostate cancer therapy to the subject identified as having prostate cancer following the prostate biopsy.

Accordingly, in some embodiments, further provided are methods useful for the prognosis, diagnosis, predication, monitoring and/or treatment of GG≥22 prostate cancer in a subject. In some embodiments, the predicting, and/or monitoring the status or outcome of GG≥2 prostate cancer includes assessing the presence of GG≥2 prostate cancer from a prostate biopsy of the subject. In some embodiments, predicting, and/or monitoring the status or outcome of GG≥2 prostate cancer comprises determining the efficacy of treatment. In some embodiments, methods disclosed herein are useful for assigning the likelihood that Grade Group ≥2 (e.g., GG≥2, GG≥3, GG≥4, or GG5) prostate cancer would be detected from a subject's prostate biopsy.

In some embodiments, the methods comprise determining, recommending to the subject or a healthcare provider of the subject or administering to the subject a therapeutic regimen. In some embodiments, the therapeutic regimen is an anti-cancer therapy. In some embodiments, the methods comprise modifying a therapeutic regimen. Modifying a therapeutic regimen can comprise increasing a therapeutic dosage, decreasing a therapeutic dosage, or terminating a therapeutic regimen.

For example, in some embodiments, the methods described herein are useful to identify a subject having GG≥22 prostate cancer. In some embodiments, the methods described herein are useful to identify subjects with an increasing risk of having GG≥2 prostate cancer detectable from a prostate biopsy. Such subjects can be administered prostate cancer therapy (e.g., one or more of surgery, radiation therapy, hormonal therapy, targeted therapy, chemotherapy, immunotherapy, radiopharmaceuticals, or bone-modifying drugs).

Conversely, in some embodiments, subjects identified as having GG<2 prostate cancer, or having a low risk of having GG≥22 prostate cancer can be given an option to forgo a biopsy or treatment and opt for watchful waiting or a minimal treatment.

In some embodiments, the GG≥2 prostate cancer therapy comprises administering a chemotherapeutic agent. Examples of chemotherapeutic agents include, without limitation, alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

In some embodiments, the biological therapy is an immune checkpoint therapy. Immune checkpoint inhibitors can target CTIA-4, PD-1, or PD-Li. Examples include, but are not limited to, ipilimumab, nivolumab, cemiplimab, avelumab, durvalumab, tremelimumab, dostarlimab, pembrolizumab, spartalizumab, and atezolizumab.

In some embodiments, the prostate cancer therapy is FDA-approved for treating prostate cancer. In some embodiments, the prostate cancer therapy is: abiraterone acetate, apalutamide, bicalutamide, cabazitaxel, casodex, darolutamide, degarelix, docetaxel, eligard, enzalutamide, erleada, firmagon, flutamide, goserelin acetate, jevtana, leuprolide acetate, Lupron depot, lutetium lu 177 vipivotide tetraxetan, Lynparza, mitoxantrone hydrochloride, nilandron, nilutamide, nubeqa, Olaparib, orgovyx, pluvicto, provenge, radium 223 dichloride, relugolix, rubraca, rucaparib camsylate, sipuleucel-t, taxotere, xofigo, xtandi, yonsa, zoladex, xytiga, or any combination thereof.

In some implementations, the methods, steps, techniques, and/or examples described herein can be performed by and/or using one or more compute devices. For example, methods described herein can be performed by a single compute device. As another example, methods described herein can be performed by multiple compute devices (e.g., where the compute devices communicate via a network).

FIG. 13 is a schematic block diagram of an example system 800 that includes a compute device 801 that can be used to implement methods described herein, according to an embodiment. The compute device 801 can be a hardware-based computing device and/or a multimedia device, such as, for example, a device, a desktop compute device, a smartphone, a tablet, a wearable device, a laptop, a server, and/or the like. The compute device 801 includes a processor 811, a memory 812 (e.g., including data storage), and a communicator 813 (e.g., operatively coupled to one another via a system bus). Where a method includes multiple compute devices, each of those multiple compute devices can be similar or identical in structure and/or function to compute device 801.

The memory 812 of the compute device 801 can be, for example, a random-access memory (RAM), a memory buffer, a hard drive, a read-only memory (ROM), an erasable programmable read-only memory (EPROM), and/or the like. The memory 812 can be configured to store, for example, data. In some instances, the memory 812 can store, for example, one or more software programs and/or code that can include instructions to cause the processor 811 to perform one or more processes, functions, and/or the like (e.g., the processes and/or functions described herein). In some embodiments, the memory 812 can include extendable storage units that can be added and used incrementally. In some implementations, the memory 812 can be a portable memory (for example, a flash drive, a portable hard disk, and/or the like) that can be operatively coupled to the processor 811. In some instances, the memory can be remotely operatively coupled with the compute device. For example, a remote database device can serve as a memory and be operatively coupled to the compute device.

The communicator 813 can be a hardware device operatively coupled to the processor 811 and memory 812 and/or software stored in the memory 812 executed by the processor 811. The communicator 813 can be, for example, a network interface card (NIC), a Wi-Fi™ module, a Bluetooth® module and/or any other suitable wired and/or wireless communication device. Furthermore, the communicator 813 can include a switch, a router, a hub and/or any other network device. The communicator 813 can be configured to connect the compute device 801 to a communication network (not shown in FIG. 13). In some instances, the communicator 813 can be configured to connect to a communication network such as, for example, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof.

In some instances, the communicator 813 can facilitate receiving and/or transmitting data or files through a communication network. In some instances, received data and/or a received file can be processed by the processor 811 and/or stored in the memory 812.

The processor 811 can be, for example, a hardware based integrated circuit (IC) or any other suitable processing device configured to run and/or execute a set of instructions or code. For example, the processor 811 can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 811 can be operatively coupled to the memory 812 through a system bus (for example, address bus, data bus and/or control bus).

In use, in some implementations the processor 811 of the compute device can receive data used in the processes, methods and/or algorithms described herein. The data can be received from a user of the compute device 801, from the memory 812, via another database and/or device (e.g., via communicator 813 and a network) and/or from any other suitable data source. The processor 811 can execute and/or implement the processes, methods and/or algorithms described herein to generate a risk score. In some implementations, for example, the processor can execute code to receive an amount of expression of each of a set of genes in a patient; normalize the amount of expression of each of the genes to an amount of expression of a reference gene to provide a normalized target gene value for each of the genes; multiply each normalized target gene value by a corresponding gene algorithm coefficient to provide a gene log it value for each of the genes; sum 1) the gene log it values and 2) an algorithm intercept value to provide a sample log it value; and obtaining a risk score as described herein. In some implementations, the processor 811 can generate a report based on the risk score and store the report in the memory 812 and/or present the report to a user (e.g., via a display of compute device 801 or by sending the report to another compute device via the communicator 813 and a network).

### EXAMPLES

The following Examples demonstrate and further illustrate certain embodiments and aspects of the present disclosure and are not to be construed as limiting the scope thereof.

### Example 1: MPS2 Clinical Validation

### Algorithm Development

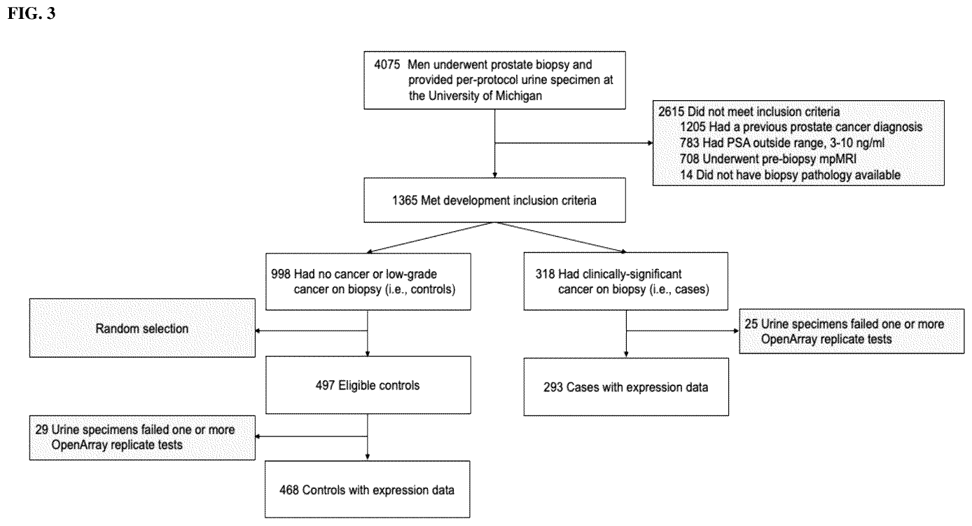

Among 815 participants, qPCR yielded valid results in 761 (93%) (FIG. 3). Power analysis demonstrated a 1:1 case to control (i.e., no cancer or grade group 1 cancer on biopsy) design with N=300 per group would provide >95% power to detect differential expression of up to 20 candidate biomarkers. Considering the incremental power gained from additional controls, 497 eligible controls were assessed with adequate urine were assessed for study inclusion.

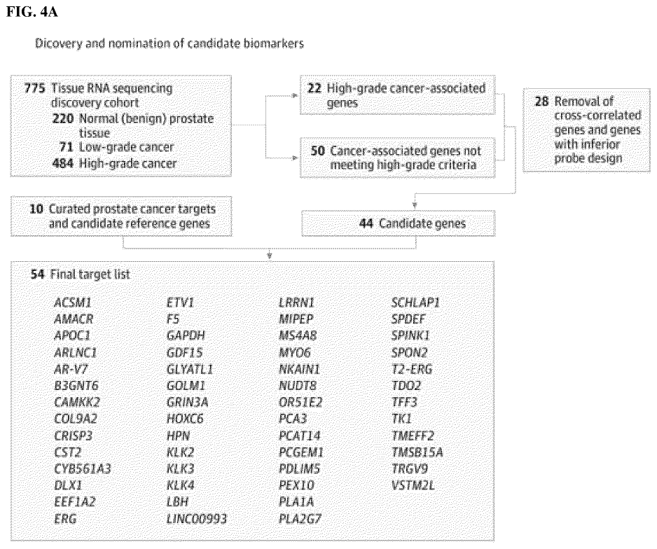

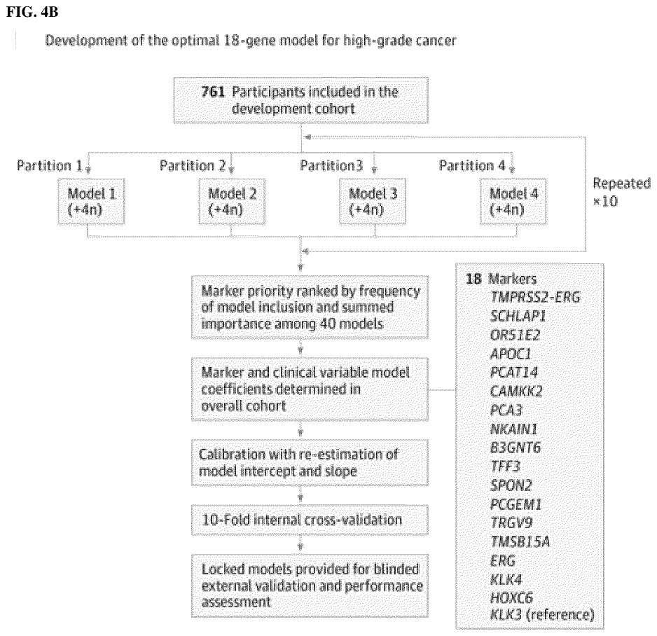

Median age was 63 years (IQR 58-68) and median PSA was 5.6 ng/mL (interquartile range (IQR) 4.6-7.2) (Table 1). On study biopsy, 293 men (39%) had GG≥2 cancer. The contribution of candidate genes to algorithm predictions was quantified across elastic net algorithms (FIGS. 4A-4B and Table 2). As shown in FIG. 4A, biomarker discovery was performed using RNA sequencing (RNAseq) data from 220 benign prostates, 71 GG1 prostate cancers, and 484 GG≥2 prostate cancers available through The Cancer Genome Atlas (TCGA), the Genotype-Tissue Expression (GTEx) portal, and the University of Michigan (U-M). Seventy-two markers met predefined criteria. Of these, qPCR probes could not be successfully designed for 19, and nine genes were highly cross correlated, resulting in exclusion from the final candidate panel. The remaining 44 transcripts meeting predefined nomination criteria were supplemented with 10 curated cancer-associated genes to yield a 54-gene candidate panel.

As demonstrated in FIG. 4B, candidate gene expression was measured by urine multiplex qPCR in the development cohort. To avoid multicollinearity in regression algorithms, highly correlated variables were identified and removed with a stepwise procedure. Specifically, variance inflation factor (VIF) was calculated for all variables (gene expression of 54 markers plus clinical factors), and the variable with the highest VIF was removed. VIF was re-calculated with remaining variables and this step was repeated until no variables remained with VIF>5. Nine probes were removed during this pre-filtering step. Three algorithm building approaches were assessed: i) logistic regression with stepwise feature selection, ii) logistic regression with recursive feature elimination, and iii) regularized logistic regression with elastic net. Performance of each algorithm-building approach was quantified as the area under the receiver-operating characteristic curve (AUC) on repeat cross validation (10-fold cross-validation repeated three times) with up-sampling of the minor class to yield balanced classes. Elastic net modeling yielded the highest median AUC and was used for development. Using an ensemble approach, which integrates data from multiple algorithms over re-samplings, the development set was randomly divided into four partitions, and the algorithm yielding the highest AUC was identified for each partition. This approach was repeated ten times with different random seeds, yielding 40 elastic net algorithms in total. The frequency of model inclusion and importance to GG≥2 prostate cancer detection was tabulated across algorithms. Based on analysis of optimal feature size and technical features of the OpenArray™ platform, the 17 biomarkers providing optimal discriminative accuracy for GG≥2 prostate cancer were included with standard clinical variables and the normalization gene KLK3 in the MPS2 and MPS2+ (plus prostate volume) algorithms. Algorithms were calibrated and internally cross-validated prior to external validation.

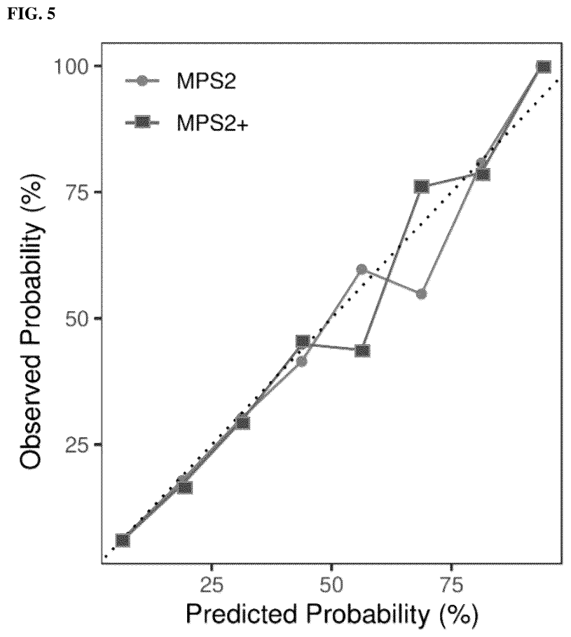

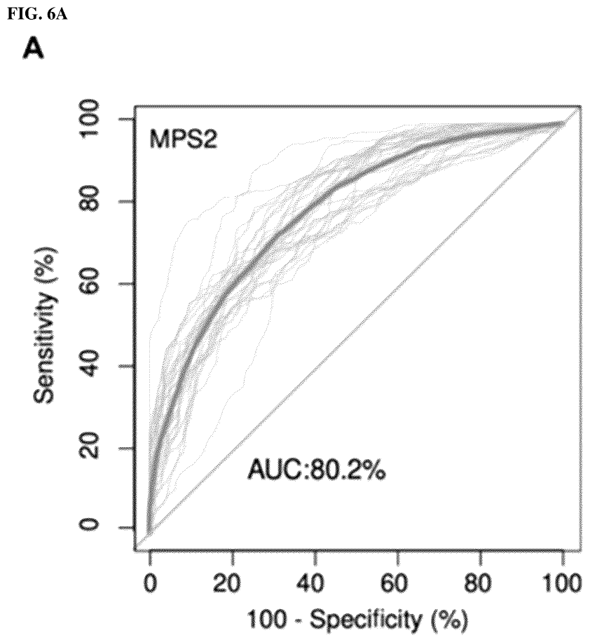

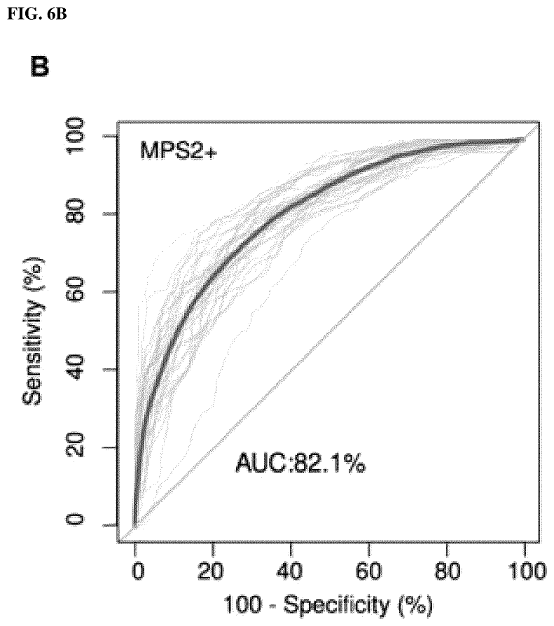

The final MPS2 algorithm included clinical variables and the 17 most informative markers, including 13 from the discovery analysis (four GG≥2-specific (APOC1, B3GNT5, NKAIN1, and SCHLAP1), nine cancer-specific (PCGEM1, SPON2, TRGV9, PCA3, OR51E2, CAMKK2, TFF3, PCAT14, and TMSB15A)), four curated markers (HOXC6, ERG, TMPRSS2:ERG, and KLK4), plus the reference gene KLK3. Algorithm coefficients were determined in the overall cohort using an elastic net regression. Calibration and internal cross validation were performed (FIG. 5 and FIGS. 6A-6B), and the MPS2 algorithms were locked for external validation. As shown in FIG. 5, the MPS2 algorithms were calibrated to account for differences in outcome prevalence between the development and validation cohorts. Two calibration methods (as described in Vergouwe et al., 2017, Statistics in medicine 36:28: 4529-4539 and Saerens et al., 2002, Neural computation 14:1:21-41, respectively) were applied to a re-sampled development set with outcome prevalence matched to the validation cohort: i) recalibration in the large, which includes re-estimation of the algorithm intercept value, and ii) logistic recalibration, which includes re-estimation of algorithm intercept value and calibration slope value. The latter method algorithm provided superior performance and was used for calibration. The calibrated algorithm was locked, and internal cross validation was performed using the train function from the R package caret (such as described in Kuhn, 2008, Journal of Statistical Software, 28(5), 1-26). Calibration was reported graphically as observed versus predicted risk of outcome. Shown are post calibration curves for MPS2 (green) and MPS2+ (blue) in the development set re-sampled to match GG≥2 prostate cancer prevalence in the validation cohort (FIG. 5). The observed prevalence of GG≥2 prostate cancer closely approximates the MPS2 and MPS2+ predicted probabilities, reflecting a good calibration.

TABLE 1
Characteristics of development and validation populations overall and stratified by pathologic findings on prostate biopsy

| | Median (IQR) | | | | | | | | | |
|---|---|---|---|---|---|---|---|---|---|---|
| | Development cohort | | | | | External validation cohort | | | | |
| | | Negative | | Negative or GGI | | | Negative | | Negative of GG1 | |
| | Total | | GGI | | GG ≥ 2 | Total | | GG1 | | GG ≥ 2 |
| Characteristic | (n = 761) | (n = 362) | (n = 106) | (n = 468) | (n = 293) | (n = 743) | (n = 452) | (n = 140) | (n = 592) | (n = 151) |
| Age, y | 63 (58-68) | 62 (57-67) | 64 (57-68) | 63 (57-68) | 64(58-69) | 62 (57-58) | 62 (57-67) | 63 (57-67) | 62 (57-68) | 64 (59-70) |
| Race, No. (%)^{a} | | | | | | | | | | |
| Black | 33 (4) | 12 (3) | 4 (4) | 16 (3) | 17 (6) | 95 (13) | 51 (11) | 19 (14) | 70 (12) | 25 (17) |
| Other race | 728 (96) | 150 (97) | 102 (96) | 452 (97) | 276 (94) | 648 (87) | 401 (89) | 123 (86) | 522 (88) | 126 (83) |
| Positive family | 206 (27) | 88 (24) | 28 (26) | 116 (25) | 90 (31) | 212 (29) | 118 (26) | 46 (33) | 164 (28) | 48 (32) |
| history, No. (%) | | | | | | | | | | |
| Previous negative | 163 (21) | 105 (29) | 22 (21) | 127 (27) | 36 (12) | 247 (33) | 196 (43) | 33 (24) | 229 (39) | 18 (12) |
| biopsy, No. (%) | | | | | | | | | | |
| Abnormal | 104 (14) | 34 (9) | 4 (4) | 38 (8) | 66 (23) | 139 (19) | 72 (16) | 16 (11) | 88 (15) | 51 (34) |
| DRE, No. (%) | | | | | | | | | | |
| Prostate | 48 (36-66) | 56 (42-76) | 47 (37-61) | 53 (40-72) | 41 (30-54) | 43 (32-60) | 48 (35-69) | 40 (28-52) | 46 (34-65) | 36 (28-47) |
| volume, mL^{b} | | | | | | | | | | |
| PSA, ng/mL | 5.6 | 5.6 | 5.535 | 5.6 | 5.6 | 5.6 | 5.5 | 5.3 | 5.4 | 6.2 |
| | (4.5-7.2) | (4.6-6.5) | (4.68-7.0) | (4.6-6.9) | (4.7-7.5) | (4.1-8.0) | (4.0-8.0) | (4.3-7.0) | (4.0-7.7) | (4.7-5.9) |
| PSA density, | 0.12 | 0.10 | 0.12 | 0.10 | 0.15 | 0.12 | 0.11 | 0.12 | 0.11 | 0.17 |
| ng/mL^{bc} | (0.08-0.16) | (0.07-0.14) | (0.09-0.16) | (0.07-0.14) | (0.10-0.20) | (0.08-0.19) | (0.07-0.17) | (0.09-0.18) | (0.08-0.17) | (0.12-0.31) |
| PHI | NA | NA | NA | NA | NA | 40.5 | 36.5 | 40.8 | 37.4 | 57.5 |
| | | | | | | (30.0-55.0) | (27.7-47.6) | (32.2-50.7) | (28.4-49.5) | (44.9-86.8) |
| Derived multiplex | NA | NA | NA | NA | NA | 0.46 | 0.40 | 0.45 | 0.47 | 0.70 |
| 2-gene model | | | | | | (0.33-0.67) | (0.28-0.59) | (0.36-0.64) | (0.31-0.60) | (0.49-0.90) |
| Derived multiplex | NA | NA | NA | NA | NA | 0.45 | 0.38 | 0.53 | 0.41 | 0.59 |
| 3-gene model | | | | | | (0.31-0.60) | (0.28-0.51) | (0.39-0.66) | (0.29-0.55) | (0.46-6.70) |
| MPS | 37 (20-58) | 26 (14-42) | 42 (24-63) | 29 (16-48) | 51 (33-72) | 35 (17-56) | 26 (12-44) | 42 (26-65) | 30 (15-49) | 35 (37-72) |
| MPS2^{d} | 0.16 | 0.07 | 0.15 | 0.07 | 0.40 | 0.13 | 0.08 | 0.20 | 0.10 | 0.44 |
| | (0.05-0.39) | (0.03-0.16) | (0.06-0.30) | (0.03-0.19) | (0.20-0.61) | (0.05-0.37) | (0.03-0.19) | (0.08-0.43) | (0.04-0.24) | (0.23-0.69) |
| MPS2^{+d} | 0.14 | 0.06 | 0.14 | 0.07 | 0.44 | 0.15 | 0.08 | 0.25 | 0.11 | 0.54 |
| | (0.05-0.42) | (0.02-0.14) | (0.06-0.32) | (0.03-0.17) | (0.22-0.68) | (0.05-0.43) | (0.03-0.21) | (0.09-0.48) | (0.04-0.30) | (0.27-0.79) |

Abbreviations: DRE, digital rectal examination; GG, grade group; MPS, MyProstateScore; MPS2, MyProstateScore 2.0, MPS2+, MyProstateScore 2.0 plus prostate volume; NA, not applicable; PHI, Prostata Health Index; PSA, prostate-specific antigen.
Si conversion factor: To convert PSA to μg/L, multiply by 1.
^{a}Race was self-reported by participants via a questionnaire. Black race was pertinent to the current study due to the well-established association of race with prostate cancer incidence, outcomes, and tumor molecular subtypes.
^{bc}The other race category includes American Indian or Alaska Native, Asian, Native Hawaiian or Other Pacific Islander, White, other, and unknown race.
^{b}Measured transrectal ultrasound.
^{c}PSA density equals serum PSA divided by prostate volume.
^{d}MPS2 and MPS2+ values are reported on a continuous scale as the likelihood of cancer of GG 2 or greater desection on biopsy.

TABLE 2
Frequency of Inclusion and Cumulative Importance of the 17 Most Informative
Markers Across Elastic Net Algorithms Assessed in Development.

| | | | | | Cumulative |
|---|---|---|---|---|---|
| No. | Gene Name | Chromosome | Gene ID | Frequency | Importance ^{a} |
| 1 | TMPRSS2-ERG | 21-21 | ENSG00000184012, | 40 | 1265 |
| | | | ENSG00000157554 | | |
| 2 | SCHLAP1 | 2 | ENSG00000281131 | 35 | 1582 |
| 3 | OR51E2 | 11 | ENSG00000167332 | 33 | 2006 |
| 4 | APOC1 | 19 | ENSG00000130208 | 31 | 456 |
| 5 | PCAT14 | 22 | ENSG00000280623 | 30 | 841 |
| 6 | CAMKK2 | 12 | ENSG00000110931 | 29 | 1604 |
| 7 | PCA3 | 9 | ENSG00000225937 | 28 | 1015 |
| 8 | NKAIN1 | 1 | ENSG00000084628 | 28 | 456 |
| 9 | B3GNT6 | 11 | ENSG00000198488 | 28 | 211 |
| 10 | TFF3 | 21 | ENSG00000160180 | 26 | 1329 |
| 11 | SPON2 | 4 | ENSG00000159674 | 26 | 1080 |
| 12 | PCGEM1 | 2 | ENSG00000227418 | 26 | 725 |
| 13 | TRGV9 | 7 | ENSG00000211695 | 24 | 955 |
| 14 | TMSB15A | X | ENSG00000158164 | 22 | 548 |
| 15 | ERG | 21 | ENSG00000157554 | 21 | 221 |
| 16 | KLK4 | 19 | ENSG00000167749 | 20 | 1094 |
| 17 | HOXC6 | 12 | ENSG00000197757 | 20 | 354 |

^{a} Cumulative importance indicates the relative weight of marker importance summed across repeat samplings as derived by elastic net modeling.

### Overall Study Population

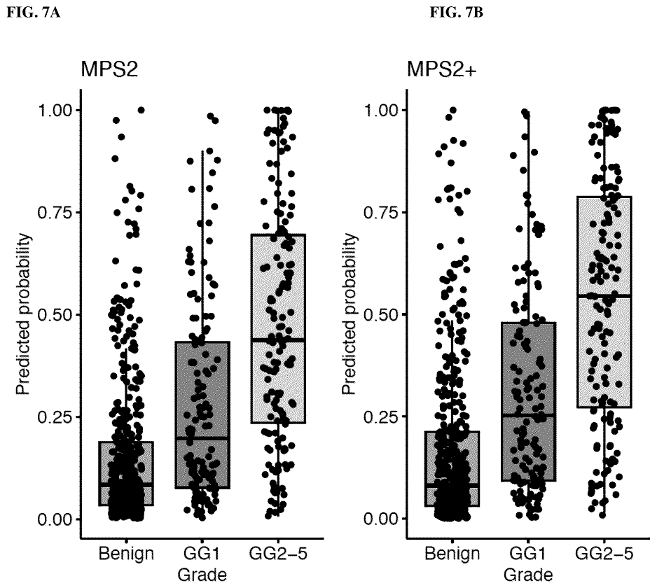

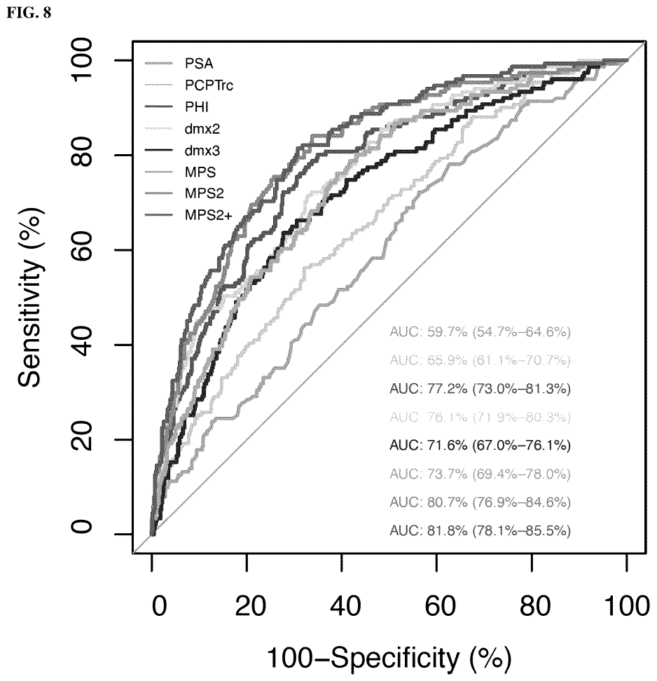

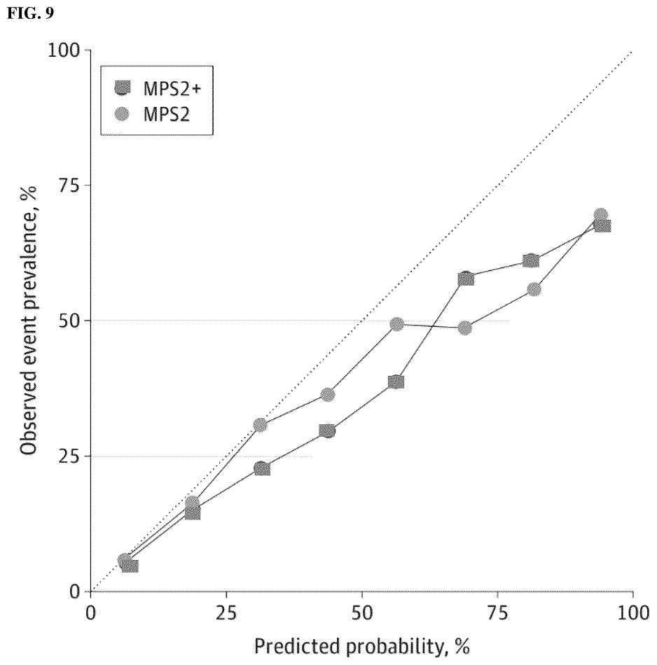

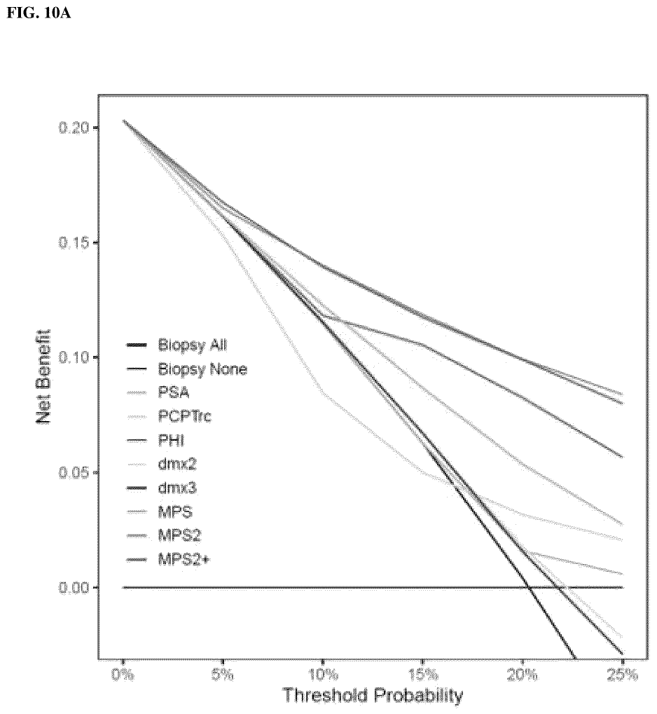

Of 813 patients, qPCR was successful in 743 (91%). Median PSA was 5.6 ng/mL (IQR 4.1-8.0), 95 men (13%) were of self-reported African-American race, and 247 men (33%) had a previous negative biopsy (Table 1). On study biopsy, 151 men (20%) had GG≥2 prostate cancer. Median MPS2 values were significantly higher in men with GG≥2 prostate cancer than in men with negative biopsies and GG01 prostate cancer (0.44 vs. 0.08 and 0.20, respectively; both p<0.001) (Table 1 and FIG. 7A). Similarly, median MPS2+ values were significantly higher in men with GG≥2 prostate cancer relative to negative biopsies or GG1 prostate cancer (0.54 vs. 0.08 and 0.25, respectively; p<0.001) (Table 1 and FIG. 7B). The AUC for GG≥2 prostate cancer was 0.60 for PSA, 0.66 for Prostate Cancer Prevention Trial Risk Calculator 2.0 (“PCPTrc”), 0.77 for Prostate Health Index (“PHI”), 0.76 for dmx2, 0.72 for dmx3, and 0.74 for MyProstateScore (“MPS”), as compared to 0.81 for MPS2 and 0.82 for MPS2+ (FIG. 8). The PSA algorithm generated prostate cancer risk scores based on a logistic regression of PSA concentration to prostate biopsy outcome. The derived multiplex 2-gene algorithm (“dmx2”) generates prostate cancer risk scores based on a logistic regression of HOXC6 and DLX 1 RNA abundance to prostate biopsy outcome. Similarly, the derived multiplex 3-gene algorithm (“dmx3”) generates prostate cancer risk scores based on a logistic regression of PCA3, ERG and SPDEF RNA abundance to prostate biopsy outcome. The observed prevalence of GG≥22 prostate cancer closely approximated MPS2 and MPS2+ risk scores (FIG. 9), reflecting good calibration. Critically, the algorithms were particularly well-calibrated for MPS2 and MPS2+ risk scores <30%. Clinical consequences of pre-biopsy biomarker testing were assessed. At 95% sensitivity, the percentages of unnecessary biopsies that were estimated to have been avoided using each test were 11% for PSA, 20% for PCPTrc, 26% for PHI, 27% for dmx2, 17% for dmx3, and 23% for MPS, as compared to 37% for MPS2 and 41% for MPS2+. Full performance measures and the estimated numbers of unnecessary biopsies avoided per 1000 patients are listed in Table 3. Critically, MPS2 and MPS2+ provided 99% sensitivity and 99% NPV for GG≥3 prostate cancer.

TABLE 3
Performance of PSA, PCPTrc, PHI, dmx2, dmx3, MPS, MPS2, and
MPS2+ in the NCI-Early Detection Research Network (“NCI-
EDRN”) Validation Cohort: Overall (N = 743), Initial
Biopsy (N = 496), and Repeat Biopsy (N = 247) Subpopulations

| | | | | | Estimated |
|---|---|---|---|---|---|
| | | | | | unnecessary |
| | | | | | biopsies |
| | | | | | avoided |
| | % | | | | per 1000 |
| Model | Sensitivity | Specificity | NPV | PPV | patients |

Overall (n = 743)

| | | | | | |
|---|---|---|---|---|---|
| PSA | 95 | 11 | 90 | 21 | 110 |
| Prostate Cancer | 95 | 20 | 94 | 23 | 198 |
| Prevention | | | | | |
| Trial risk calculator | | | | | |
| PHI | 95 | 26 | 96 | 25 | 260 |
| Derived multiplex | 95 | 27 | 96 | 25 | 271 |
| 2-gene model | | | | | |
| Derived multiplex | 95 | 17 | 94 | 23 | 171 |
| 3-gene model | | | | | |
| MPS | 95 | 23 | 94 | 24 | 231 |
| MPS2 | 95 | 37 | 97 | 28 | 375 |
| MPS2+ | 95 | 41 | 97 | 29 | 406 |

Initial biopsy (n = 496)

| | | | | | |
|---|---|---|---|---|---|
| PSA | 95 | 15 | 89 | 29 | 154 |
| Prostate Cancer | 95 | 27 | 94 | 32 | 267 |
| Prevention | | | | | |
| Trial risk calculator | | | | | |
| PHI | 95 | 30 | 95 | 33 | 300 |
| Derived multiplex | 95 | 30 | 95 | 33 | 300 |
| 2-gene model | | | | | |
| Derived multiplex | 95 | 17 | 91 | 30 | 169 |
| 3-gene model | | | | | |
| MPS | 95 | 27 | 93 | 32 | 272 |
| MPS2 | 95 | 35 | 95 | 35 | 347 |
| MPS2+ | 95 | 42 | 96 | 37 | 419 |

Repeat biopsy (n = 247)

| | | | | | |
|---|---|---|---|---|---|
| PSA | 94.4 | 15 | 97 | 8.0 | 148 |
| Prostate Cancer | 94.4 | 21 | 98 | 8.6 | 211 |
| Prevention | | | | | |
| Trial risk calculator | | | | | |
| PHI | 94.4 | 8.7 | 95 | 7.5 | 87 |
| Derived multiplex | 94.4 | 14 | 97 | 8.0 | 142 |
| 2-gene model | | | | | |
| Derived multiplex | 94.4 | 16 | 97 | 8.1 | 162 |
| 3-gene model | | | | | |
| MPS | 94.4 | 15 | 97 | 8.0 | 148 |
| MPS2 | 94.4 | 46 | 99 | 12 | 463 |
| MPS2+ | 94.4 | 51 | 99 | 13 | 511 |

### Repeat Biopsy Subpopulation

The repeat biopsy population included 247 men with median PSA 7.2 ng/mL (IQR 55-9.8), of which 18 (7.3%) were found to have GG≥2 prostate cancer (Table 4). At 95% sensitivity, the proportions of unnecessary biopsies estimated to have been avoided were 15% for PSA, 8.7% for PHI, 14% for dmx2, 16% for dmx3, and 15% for MPS, as compared to 46% for MPS2 and 51% for MPS2+ (Table 3). Accordingly, MPS2 testing was estimated to have avoided approximately one-half of unnecessary biopsies while maintaining detection of 95% of GG≥2 prostate cancers. The performance metrics of MPS2 biomarker only, MPS2 algorithm and MPS2+ algorithm on the repeat biopsy population are found in Table 5.

TABLE 4
Characteristics of the NCI-EDRN External Validation
Population Stratified by Previous Biopsy Status

| | Initial Biopsy | Repeat Biopsy |
|---|---|---|
| Characteristic | (N = 496) | (N = 247) |
| Median age (IQR) - years | 62 (56-67) | 63 (59-68) |
| African-American ^{a}- No. (%) | 70 (14%) | 25 (10%) |
| Positive family history - No. (%) | 134 (27%) | 78 (32%) |
| Previous negative biopsy - No. (%) | 0 (0%) | 247 (100%) |
| Abnormal DRE - No. (%) | 111 (22%) | 28 (11%) |
| Median prostate volume ^{b} - mL | 40 (29-51) | 56 (39-81) |
| Median PSA (IQR) - ng/mL | 5.0 (3.8-6.6) | 7.2 (5.5-9.8) |
| Median PSA density ^{c} | 0.12 (0.08-0.19) | 0.12 (0.09-0.20) |
| (IQR) - ng/mL² | | |
| Median PHI (IQR) | 40.8 (30.2-55.1) | 39.3 (29.6-54.7) |
| Median PCA3 (IQR) | 27.4 (12.4-61.1) | 21.2 (10.0-45.1) |
| Median MPS (IQR) | 34 (16-57) | 35 (17-55) |
| Median MPS2 ^{d} (IQR) | 0.20 (0.08-0.46) | 0.06 (0.02-0.14) |
| Median MPS2+ ^{d} (IQR) | 0.22 (0.08-0.53) | 0.06 (0.02, 0.21) |
| Biopsy GG ≥ 2 - No. (%) | 133 (27%) | 18 (7.3%) |

Abbreviations: DRE, digital rectal examination; GG, denotes grade group; IQR, interquartile range; MPS, MyProstateScore; MPS2, MyProstateScore 2.0; MPS2+, MyProstateScore 2.0 plus; PCA3, prostate cancer antigen 3; PHI, prostate health index; PSA, prostate-specific antigen
^{a} African-American race was self-reported by participants. Race was assessed due to the well-established association of race with prostate cancer incidence and outcomes.
^{b} Measured by transrectal ultrasound.
^{c} PSA density equals serum PSA divided by prostate volume.
^{d} MPS2 and MPS2+ values are reported on a continuous scale as the likelihood of detecting clinically significant prostate cancer on biopsy.

TABLE 5
Clinical Performance of High Sensitivity MPS2 Threshold
Values in the Repeat Biopsy Subpopulation of the
External Validation Cohort (N = 247).

| Threshold | Sensitivity | Specificity | NPV | PPV |
|---|---|---|---|---|
| MPS2+ | | | | |
| 0.04 | 100% | 40% | 100% | 12% |
| 0.05 | 94% | 47% | 99% | 12% |
| 0.058 | 94% | 51% | 99% | 13% |
| 0.06 | 88% | 52% | 98% | 13% |
| 0.07 | 83% | 57% | 98% | 13% |
| 0.08 | 78% | 59% | 97% | 13% |
| 0.09 | 67% | 62% | 96% | 12% |
| 0.10 | 67% | 62% | 96% | 12% |
| MPS2 | | | | |
| 0.04 | 94% | 42% | 99% | 11% |
| 0.044 | 94% | 46% | 99% | 12% |
| 0.05 | 89% | 48% | 98% | 12% |
| 0.06 | 89% | 52% | 98% | 13% |
| 0.07 | 72% | 57% | 96% | 12% |
| 0.08 | 67% | 62% | 96% | 12% |
| 0.09 | 67% | 66% | 96% | 13% |
| 0.10 | 67% | 68% | 96% | 14% |
| Biomarkers | | | | |
| Only | | | | |
| 0.04 | 100% | 22% | 100% | 9.1% |
| 0.05 | 100% | 26% | 100% | 9.6% |
| 0.06 | 100% | 32% | 100% | 10% |
| 0.07 | 100% | 35% | 100% | 11% |
| 0.08 | 94% | 42% | 99% | 11% |
| 0.09 | 83% | 45% | 97% | 11% |
| 0.10 | 78% | 49% | 97% | 11% |

### Initial Biopsy Subpopulation

The initial biopsy population included 496 patients with median PSA 5.0 ng/mL (IQR 3.8-6.6) (Table 4). On study biopsy, 133 (27%) had GG≥2 cancer. Evaluating each algorithm at 95% sensitivity, the proportions of unnecessary biopsies avoided were 15% for PSA, 27% for PCPT, 30% for PHI, 30% for dmx2, 17% for dmx3, and 27% for MPS, as compared to 35% for MPS2 (Table 3). Although prostate volume data were not always available for initial biopsy patients, use of MPS2+ would have avoided 42% of unnecessary biopsies. Performance of MPS2 algorithms with and without clinical factors are provided by subpopulation (Table 6).

TABLE 6
Clinical Performance of High Sensitivity MPS2 Threshold
Values in the Initial Biopsy Subpopulation of
the External Validation Cohort (N = 496).

| Threshold | Sensitivity | Specificity | NPV | PPV |
|---|---|---|---|---|
| MPS2+ | | | | |
| 0.05 | 97% | 21% | 95% | 31% |
| 0.06 | 97% | 25% | 96% | 32% |
| 0.07 | 96% | 29% | 95% | 33% |
| 0.08 | 96% | 31% | 96% | 34% |
| 0.08 | 95% | 32% | 95% | 34% |
| 0.09 | 95% | 35% | 95% | 35% |
| 0.10 | 95% | 38% | 95% | 36% |
| 0.11 | 95% | 42% | 96% | 37% |
| 0.12 | 92% | 42% | 94% | 38% |
| 0.13 | 92% | 48% | 95% | 39% |
| 0.14 | 92% | 51% | 94% | 41% |
| 0.15 | 90% | 53% | 94% | 41% |
| 0.18 | 88% | 57% | 93% | 43% |
| 0.20 | 86% | 60% | 92% | 44% |
| MPS2 | | | | |
| 0.05 | 96% | 21% | 94% | 31% |
| 0.06 | 96% | 25% | 95% | 32% |
| 0.075 | 96% | 28% | 95% | 33% |
| 0.075 | 96% | 31% | 96% | 34% |
| 0.08 | 95% | 33% | 94% | 34% |
| 0.087 | 95% | 35% | 95% | 35% |
| 0.09 | 94% | 36% | 94% | 35% |
| 0.10 | 94% | 39% | 95% | 36% |
| 0.11 | 93% | 41% | 94% | 37% |
| 0.12 | 92% | 46% | 94% | 38% |
| 0.13 | 91% | 49% | 94% | 39% |
| 0.14 | 89% | 52% | 93% | 41% |
| 0.15 | 89% | 54% | 93% | 41% |
| 0.175 | 86% | 59% | 92% | 44% |
| 0.20 | 83% | 63% | 91% | 45% |
| Biomarkers | | | | |
| Only | | | | |
| 0.05 | 95% | 20% | 92% | 30% |
| 0.06 | 95% | 28% | 94% | 33% |
| 0.07 | 95% | 33% | 94% | 34% |
| 0.075 | 95% | 35% | 95% | 35% |
| 0.077 | 95% | 35% | 95% | 35% |
| 0.08 | 94% | 37% | 94% | 35% |
| 0.09 | 92% | 41% | 93% | 36% |
| 0.10 | 90% | 44% | 92% | 37% |
| 0.11 | 88% | 46% | 92% | 38% |
| 0.12 | 85% | 50% | 90% | 38% |
| 0.15 | 75% | 59% | 87% | 40% |
| 0.175 | 72% | 66% | 86% | 44% |
| 0.20 | 68% | 71% | 86% | 46% |

### Decision Curve Analysis

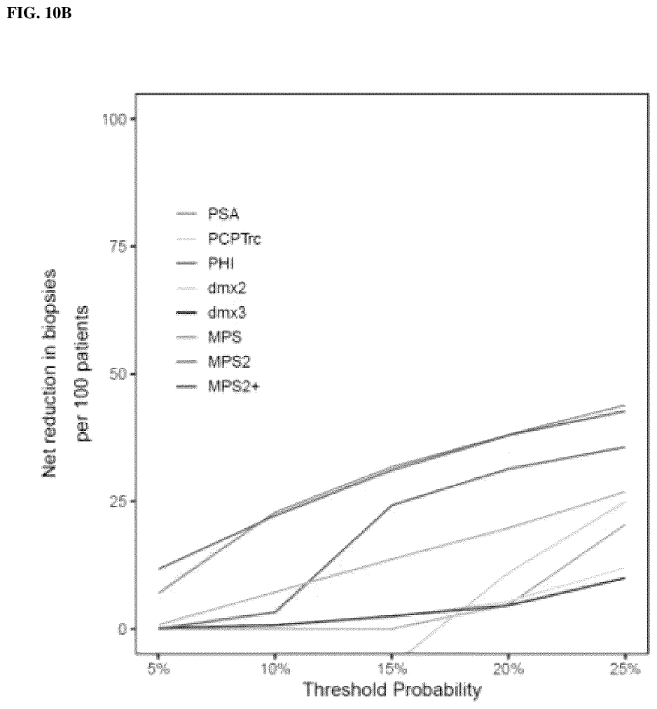

Decision curve analysis (DCA) was used to evaluate the net benefit of biomarker testing relative to “biopsy all” and “biopsy none” approaches. As shown in FIG. 10A, MPS2 and MPS2+ algorithms provided the highest net benefit across the range of clinically pertinent risk threshold values (5-20%). the risk threshold value (indicated as “threshold probability”) (x-axis) reflects how the patient and clinician value potential clinical outcomes. For example, a risk threshold value of 5% applies to patients that would choose to pursue a prostate biopsy if their risk of GG≥2 prostate cancer is 5% or higher. For GG≥2 prostate cancer, a 5% risk threshold value represents a risk-averse population, such as younger men with a long life-expectancy. At a practice level, this implies that the clinician would be willing to perform as many as 20 biopsies to detect an additional GG≥2 prostate cancer. At the other end of the spectrum, a risk threshold value of 20% applies to patients that who would choose to pursue a prostate biopsy only if their risk of GG≥2 prostate cancer was 20%. Such a population strongly values avoiding biopsy and is willing to accept a higher risk of delayed detection of GG≥2 prostate cancer. The unit of net benefit (y-axis) is number of true positives. A net benefit of 0.15 is equivalent to an approach in which 15 patients per 100 are directed to biopsy based on use of the test, and all 15 patients are found to have GG≥2 cancer.

Across the clinically pertinent risk threshold values spanning 5% to 20%, MPS2 algorithms were estimated to provide the highest net clinical benefit across all tests (FIG. 10A). Expressing benefit as net reduction in unnecessary prostate biopsies, MPS2 was estimated to have the greatest net reduction in unnecessary prostate biopsies without failing to biopsy a single patient with GG≥2 prostate cancer (FIG. 10B).

### Example 2: iMPS2Clinical Validation

The initial prostate biopsy population included 496 patients with median PSA 5.0 ng/mL (IQR 3.8-6.6) (Table 7). On study biopsy, 133 (27%) had GG≥2prostate cancer. An alternative initial biopsy algorithm (7iMPS2) was developed in a similar manner as MPS2 except it was developed exclusively from the initial biopsy population (Table 8). The same 17 biomarkers in MPS2 were retained in iMPS2. iMPS2 was developed with the same strategy as MPS2 to have three forms of the algorithm. 1) iMPS2biomarkers only algorithm; 2) iMPS2biomarkers algorithm+clinical factors algorithm (iMPS2); and 3) iMPS2 biomarkers+clinical factors+prostate volume algorithm (iMPS2+).

TABLE 7
Characteristics of the NCI-EDRN External Validation Population
Stratified by Previous Prostate Biopsy Status

| | Initial Blopsy | Repeat Biopsy |
|---|---|---|
| Characteristic | (N = 496) | (N = 247) |
| Median age (IQR) - years | 62 (56-67) | 63 (59-68) |
| African-American ^{a}- No. (%) | 70 (14%) | 25 (10%) |
| Positive family history - No. (%) | 134 (27%) | 78 (32%) |
| Previous negative biopsy - No. (%) | 0 (0%) | 247 (100%) |
| Abnormal DRE - No. (%) | 111 (22%) | 28 (11%) |
| Median prostate volume ^{b} - mL | 40 (29-51) | 56 (39-81) |
| Median PSA (IQR) - ng/mL | 5.0 (3.8-6.6) | 7.2 (5.5-9.8) |
| Median PSA density ^{c} | 0.12 (0.08-0.19) | 0.12 (0.09-0.20) |
| (IQR) - ng/mL² | | |
| Median PHI (IQR) | 40.8 (30.2-55.1) | 39.3 (29.6-54.7) |
| Median PCA3 (IQR) | 27.4 (12.4-61.1) | 21.2 (10.0-45.1) |
| Median MPS (IQR) | 34 (16-57) | 35 (17-55) |
| Median MPS2 ^{d} (IQR) | 0.20 (0.08-0.46) | 0.06 (0.02-0.14) |
| Median MPS2+ ^{d} (IQR) | 0.22 (0.08-0.53) | 0.06 (0.02, 0.21) |
| Biopsy GG ≥ 2 - No. (%) | 133 (27%) | 18 (7.3%) |

Abbreviations: DRE, digital rectal examination; GG, denotes grade group; IQR, interquartile range; MPS, MyProstateScore; MPS2, MyProstateScore 2.0; MPS2+, MyProstateScore 2.0 plus; PCA3, prostate cancer antigen 3; PHI, prostate health index; PSA, prostate-specific antigen
^{a} African-American race was self-reported by participants. Race was assessed due to the well-established association of race with prostate cancer incidence and outcomes.
^{b} Measured by transrectal ultrasound.
^{c} PSA density equals serum PSA divided by prostate volume.
^{d} MPS2 and MPS2+ values are reported on a continuous scale as the likelihood of detecting clinically significant prostate cancer on biopsy.

TABLE 8
Characteristics of the Development Cohort for Patients Undergoing
Initial Prostate Biopsy used to Develop iMPS2

| | Initial Biopsy |
|---|---|
| Characteristic | (N = 598) |
| Median age (IQR) - years | 63 (57-58) |
| African-American ^{a}- No. (%) | 24 (4.0%) |
| Positive family history - No. (%) | 157 (26%) |
| Previous negative biopsy - No. (%) | 0 (0%) |
| Abnormal DRE - No. (%) | 87 (15%) |
| Median prostate volume ^{b} - mL | 36 (35-61) |
| Median PSA (IQR) - ng/mL | 5.4 (4.5-6.8) |
| Median PSA density ^{c} (IQR) - ng/mL² | 0.12 (0.08-0.16) |
| Median MPS2 ^{d} (IQR) | 0.20 (0.08-0.46) |
| Median MPS2+ ^{d} (IQR) | 0.22 (0.08-0.53) |
| Biopsy GG ≥ 2 - No. (%) | 133 (27%) |

^{a} African-American race was self-reported by participants. Race was assessed due to the well-established association of race with prostate cancer incidence and outcomes.
^{b} Measured by transrectal ultrasound.
^{c} PSA density equals serum PSA divided by prostate volume.
^{d} MPS2 and MPS2+ values are reported on a continuous scale as the likelihood of detecting clinically significant prostate cancer on biopsy.

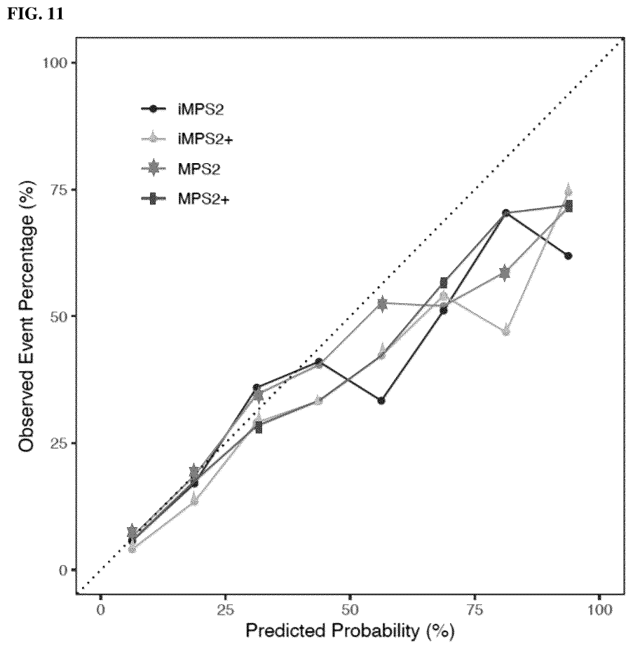

iMPS2 was similarly validated as MPS2 on the NCI-EDRN cohort. iMPS2 performance across risk thresholds 0.05-0.25 were evaluated and algorithm performance was measured for sensitivity, specificity, NPV and PPV (Table 9). The observed prevalence of GG≥2 cancer closely approximated iMPS2 and iMPS2+ as well as MPS2 and MPS2+ risk scores (FIG. 11), reflecting good calibration.

TABLE 9
Clinical Performance of Secondary Initial Prostate Biopsy
(iMPS2) Algorithms in the Initial Prostate Biopsy Subpopulation
of the External Validation Cohort (N = 496)

| Threshold | Sensitivity | Specificity | NPV | PPV |
|---|---|---|---|---|
| iMPS2+ | | | | |
| 0.05 | 99% | 14% | 96% | 30% |
| 0.095 | 96% | 31% | 95% | 34% |
| 0.115 | 96% | 36% | 96% | 35% |
| 0.135 | 95% | 43% | 96% | 38% |
| 0.145 | 93% | 46% | 95% | 38% |
| 0.16 | 92% | 48% | 94% | 40% |
| 0.19 | 92% | 53% | 95% | 42% |
| 0.20 | 90% | 55% | 94% | 42% |
| 0.25 | 86% | 63% | 92% | 46% |
| iMPS2 | | | | |
| 0.05 | 97% | 18% | 94% | 30% |
| 0.095 | 96% | 33% | 95% | 34% |
| 0.115 | 95% | 39% | 95% | 36% |
| 0.135 | 93% | 46% | 95% | 39% |
| 0.145 | 92% | 48% | 94% | 39% |
| 0.16 | 90% | 53% | 94% | 41% |
| 0.19 | 87% | 56% | 92% | 42% |
| 0.20 | 86% | 59% | 92% | 43% |
| 0.25 | 80% | 65% | 90% | 46% |
| Biomarkers | | | | |
| Only | | | | |
| 0.05 | 96% | 15% | 92% | 29% |
| 0.095 | 93% | 35% | 93% | 34% |
| 0.115 | 91% | 40% | 92% | 36% |
| 0.135 | 90% | 46% | 92% | 38% |
| 0.145 | 90% | 49% | 93% | 39% |
| 0.16 | 86% | 53% | 91% | 40% |
| 0.19 | 80% | 60% | 90% | 43% |
| 0.20 | 77% | 63% | 88% | 44% |
| 0.25 | 72% | 69% | 87% | 46% |

### Example 3: Determination of a 17-Gene Panel for Urine Test for GG≥2Prostate Cancer Biomarker Discovery

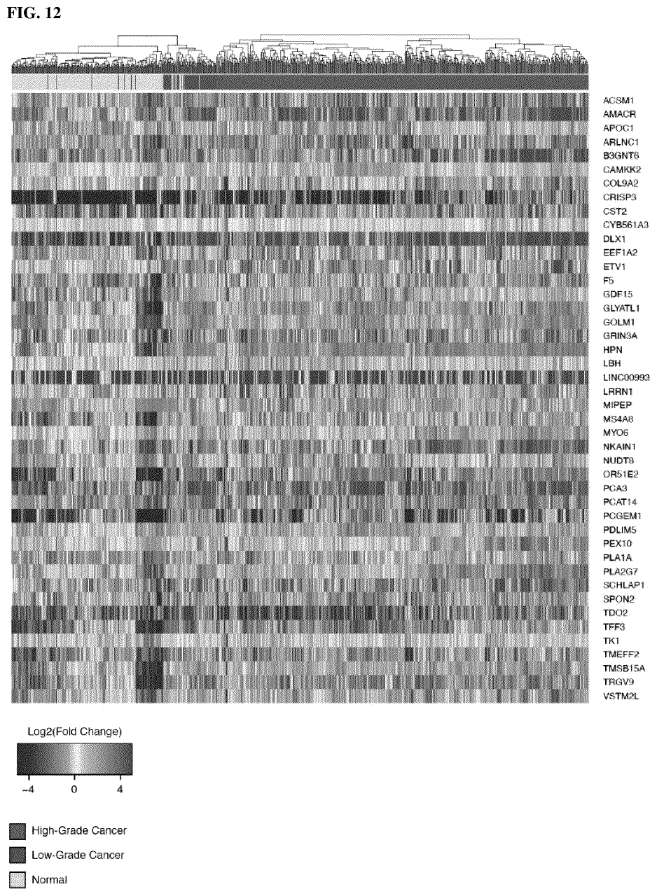

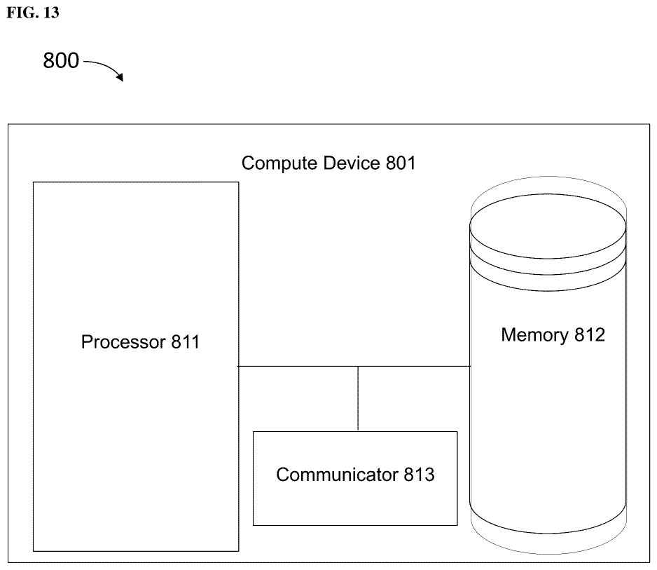

The MyProstateScore (“MPS”) test incorporates prostate cancer antigen 3 (PCA3) and TMPRSS2:ERG gene fusion expression with serum PSA level to estimate risk of GG≥2 prostate cancer. To derive agene panel for GG≥2 prostate cancer, a differential expression analysis of 58,724 genetic targets in multi-institutional RNA sequencing data was performed (FIGS. 4A-4B). A total of 72 genes met predefined nomination criteria for prostate cancer (n=50) or for particularly GG≥2 prostate cancer (n=22). Removal of collinear genes and those without PCR primers resulted in 4 candidate markers, which were supplemented with 10 previously described prostate cancer-associated or reference genes, yielding a 54-gene candidate panel (Table 10 and FIG. 12).

TABLE 10
Information for the 54 genes included in the OpenArray assay.

| gene_id | seqnames | start | end | strand | gene_name | gene_type | probes |
|---|---|---|---|---|---|---|---|
| ENSG00000157911 | chr1 | 2403964 | 2412571 | − | PEX10 | protein_coding | Hs00159991_m1 |
| ENSG00000084628 | chr1 | 31179745 | 31239554 | − | NKAIN1* | protein_coding | Hs01563334_m1 |
| ENSG00000049089 | chr1 | 40300487 | 40317294 | − | COL9A2 | protein_coding | Hs00895570_m1 |
| ENSG00000198734 | chr1 | 169514166 | 169586588 | − | F5 | protein_coding | Hs00914112_m1 |
| ENSG00000213626 | chr2 | 30231531 | 30323730 | + | LBH | protein_coding | Hs00368853_m1 |
| ENSG00000144355 | chr2 | 172085226 | 172089677 | + | DLX1 | protein_coding | Hs00698288_m1 |
| ENSG00000281131 | chr2 | 180692104 | 180916939 | + | SCHLAP1* | non_coding | Hs04968419_m1 |
| ENSG00000227418 | chr2 | 192749845 | 192776899 | + | PCGEM1* | non_coding | Hs01369007_m1 |
| ENSG00000144339 | chr2 | 191949043 | 192195709 | − | TMEFF2 | protein_coding | Hs01086905_m1 |
| ENSG00000175928 | chr3 | 3799437 | 3847703 | + | LRRN1 | protein_coding | Hs04972436_m1 |
| ENSG00000144837 | chr3 | 119597842 | 119629811 | + | PLA1A | protein_coding | Hs01056914_m1 |
| ENSG00000163110 | chr4 | 94451857 | 94668227 | + | PDLIM5 | protein_coding | Hs00935065_m1 |
| ENSG00000151790 | chr4 | 155903695 | 155920406 | + | TDO2 | protein_coding | Hs01045945_m1 |
| ENSG00000159674 | chr4 | 1166932 | 1208962 | − | SPON2* | protein_coding | Hs00202813_m1 |
| ENSG00000242110 | chr5 | 33986178 | 34008108 | − | AMACR | protein_coding | Hs01091294_m1 |
| ENSG00000164266 | chr5 | 147824568 | 147831786 | − | SPINK1 | protein_coding | Hs01004508_m1 |
| ENSG00000196586 | chr6 | 75749192 | 75919537 | + | MYO6 | protein_coding | Hs04984345_m1 |
| ENSG00000124664 | chr6 | 34537802 | 34556333 | − | SPDEF | protein_coding | Hs00171942_m1 |
| ENSG00000146070 | chr6 | 46704201 | 46735693 | − | PLA2G7 | protein_coding | Hs00965834_m1 |
| ENSG00000096006 | chr6 | 49727384 | 49744437 | − | CRISP3 | protein_coding | Hs00195988_m1 |
| ENSG00000006468 | chr7 | 13891228 | 13991425 | − | ETV1 | protein_coding | Hs00951941_m1 |
| ENSG00000211695 | chr7 | 38317017 | 38318861 | − | TRGV9* | non_coding | Hs00233330_m1 |
| | | | | | | | Hs01379483_g1 |
| ENSG00000225937 | chr9 | 76764436 | 76787569 | + | PCA3* | non_coding | Hs03309852_g1 |
| | | | | | | | Hs01371939_g1 |
| ENSG00000135052 | chr9 | 86026146 | 86099558 | − | GOLM1 | protein_coding | Hs00895846_m1 |
| ENSG00000198785 | chr9 | 101569353 | 101738580 | − | GRIN3A | protein_coding | Hs01077968_m1 |
| ENSG00000235687 | chr10 | 37309185 | 37347029 | + | LINC00993 | non_coding | Hs00418492_m1 |
| ENSG00000166840 | chr11 | 58927701 | 58957043 | + | GLYATL1 | protein_coding | Hs01014189_m1 |
| ENSG00000166959 | chr11 | 60699574 | 60715811 | + | MS4A8 | protein_coding | Hs00230227_m1 |
| ENSG00000198488 | chr11 | 77034398 | 77041973 | + | B3GNT6* | protein_coding | Hs00934529_s1 |
| ENSG00000167332 | chr11 | 4680171 | 4697842 | − | OR51E2* | protein_coding | Hs04231197_m1 |
| ENSG00000162144 | chr11 | 61348745 | 61362299 | − | CYB561A3 | protein_coding | Hs04401125_m1 |
| ENSG00000167799 | chr11 | 67627938 | 67629930 | − | NUDT8 | protein_coding | Hs00378190_m1 |
| ENSG00000111640 | chr12 | 6533927 | 6538374 | + | GAPDH | protein_coding | Hs99999905_m1 |
| ENSG00000197757 | chr12 | 54015897 | 54030823 | + | HOXC6* | protein_coding | Hs00171690_m1 |
| ENSG00000110931 | chr12 | 121237691 | 121298308 | − | CAMKK2* | protein_coding | Hs00902176_m1 |
| ENSG00000027001 | chr13 | 23730189 | 23889419 | − | MIPEP | protein_coding | Hs00969246_m1 |
| ENSG00000166743 | chr16 | 20623237 | 20691256 | − | ACSM1 | protein_coding | Hs01048213_m1 |
| ENSG00000260896 | chr16 | 80828735 | 80892595 | − | PRCAT47 | non_coding | Hs01596342_m1 |
| ENSG00000167900 | chr17 | 78174075 | 78187233 | − | TK1 | protein_coding | Hs00177406_m1 |
| ENSG00000130513 | chr19 | 18386158 | 18389176 | + | GDF15 | non_coding | Hs00171132_m1 |
| ENSG00000105707 | chr19 | 35040506 | 35066571 | + | HPN | protein_coding | Hs00170096_m1 |
| ENSG00000130208 | chr19 | 44914247 | 44919346 | + | APOC1* | protein_coding | Hs00155790_m1 |
| ENSG00000142515 | chr19 | 50854915 | 50860764 | + | KLK3* | protein_coding | Hs03063374_m1 |
| ENSG00000167751 | chr19 | 50873249 | 50880567 | + | KLK2 | protein_coding | Hs00428383_m1 |
| ENSG00000167749 | chr19 | 50906352 | 50910738 | − | KLK4* | protein_coding | Hs00191772_m1 |
| ENSG00000132821 | chr20 | 37903104 | 37945350 | + | VSTM2L | protein_coding | Hs01067452_m1 |
| ENSG00000170369 | chr20 | 23823769 | 23826731 | − | CST2 | protein_coding | Hs00606913_m1 |
| ENSG00000101210 | chr20 | 63488013 | 63499315 | − | EEF1A2 | protein_coding | Hs00951279_m1 |
| ENSG00000157554 | chr21 | 38380027 | 38661780 | − | ERG | protein_coding | Hs01554635_m1 |
| ENSG00000160180 | chr21 | 42311667 | 42315651 | − | TFF3* | protein_coding | Hs00902278_m1 |
| ENSG00000280623 | chr22 | 23536881 | 23547797 | + | PCAT14* | non_coding | Hs04941925_m1 |
| ENSG00000169083 | chrX | 67544032 | 67730619 | + | AR | protein_coding | Hs04260217_m1 |
| ENSG00000158164 | chrX | 102513676 | 102516784 | − | TMSB15A* | protein_coding | Hs00762927_s1 |
| ENSG00000184012 | | | | | TMPRSS2- | fusion | Hs03063375_ft |
| ENSG00000157554 | | | | | ERG* | | |

Information for the 54 genes included in the OpenArray assay.

| | High- vs Low- Grade | | High-Grade vs Benign | | | |
|---|---|---|---|---|---|---|
| gene_id | logFC | adj. P. Val | logFC | adj. P. Val | AUC | note |
| ENSG00000157911 | 0.52 | 1.11E−01 | 0.95 | 2.76E−06 | 0.567 | |
| ENSG00000084628 | 0.98 | 1.60E−01 | 1.77 | 8.79E−05 | 0.500 | |
| ENSG00000049089 | 0.35 | 5.12E−01 | 1.28 | 1.02E−05 | 0.546 | |
| ENSG00000198734 | 0.73 | 1.72E−01 | 1.64 | 1.20E−05 | 0.550 | |
| ENSG00000213626 | 0.65 | 1.28E−02 | 0.60 | 7.03E−04 | 0.560 | |
| ENSG00000144355 | 0.65 | 3.56E−01 | 5.72 | 3.26E−19 | 0.550 | |
| ENSG00000281131 | 2.00 | 3.13E−02 | 1.85 | 2.02E−03 | 0.638 | |
| ENSG00000227418 | −1.05 | 1.56E−01 | 0.71 | 2.39E−01 | 0.494 | |
| ENSG00000144339 | −0.36 | 5.96E−01 | 1.07 | 2.46E−03 | 0.541 | |
| ENSG00000175928 | 0.72 | 1.49E−01 | 0.74 | 1.42E−02 | 0.481 | |
| ENSG00000144837 | −0.18 | 7.22E−01 | 1.05 | 1.25E−04 | 0.501 | |
| ENSG00000163110 | 0.26 | 4.38E−01 | 1.69 | 4.73E−21 | 0.612 | |
| ENSG00000151790 | 0.72 | 4.25E−01 | 0.08 | 8.80E−01 | 0.498 | |
| ENSG00000159674 | 0.29 | 6.68E−01 | 2.17 | 1.20E−11 | 0.597 | |
| ENSG00000242110 | −0.07 | 9.10E−01 | 2.99 | 1.27E−30 | 0.577 | |
| ENSG00000164266 | −0.48 | 6.22E−01 | 1.46 | 9.96E−03 | 0.519 | |
| ENSG00000196586 | 0.32 | 2.58E−01 | 0.90 | 5.77E−08 | 0.560 | |
| ENSG00000124664 | −0.11 | 6.21E−01 | 1.88 | 1.74E−24 | 0.570 | |
| ENSG00000146070 | 0.34 | 4.03E−01 | 1.56 | 3.84E−10 | 0.560 | |
| ENSG00000096006 | 0.98 | 4.14E−01 | 1.58 | 2.89E−02 | 0.521 | |
| ENSG00000006468 | 0.85 | 9.96E−02 | −0.20 | 5.10E−01 | 0.565 | |
| ENSG00000211695 | −0.16 | 7.95E−01 | 2.96 | 6.05E−14 | 0.642 | |
| ENSG00000225937 | −0.76 | 3.69E−01 | 4.64 | 6.76E−12 | 0.685 | original |
| | | | | | | MPS gene |
| ENSG00000135052 | −0.16 | 6.35E−01 | 2.04 | 1.78E−33 | 0.570 | |
| ENSG00000198785 | 1.07 | 9.73E−02 | 1.14 | 4.59E−03 | 0.531 | |
| ENSG00000235687 | 0.77 | 3.69E−01 | 1.92 | 9.37E−05 | 0.526 | |
| ENSG00000166840 | −0.52 | 2.35E−01 | 2.37 | 8.36E−11 | 0.579 | |
| ENSG00000166959 | −0.02 | 9.78E−01 | 1.78 | 2.15E−07 | 0.623 | |
| ENSG00000198488 | 2.03 | 3.98E−02 | 2.93 | 1.28E−05 | 0.564 | |
| ENSG00000167332 | −0.84 | 1.61E−01 | 2.84 | 4.44E−15 | 0.642 | |
| ENSG00000162144 | 0.27 | 2.09E−01 | 0.66 | 8.83E−08 | 0.556 | |
| ENSG00000167799 | −0.28 | 5.25E−01 | 1.38 | 4.14E−07 | 0.615 | |
| ENSG00000111640 | 0.10 | 5.14E−01 | −0.02 | 8.22E−01 | 0.530 | alternative |
| | | | | | | reference |
| | | | | | | gene |
| ENSG00000197757 | 0.58 | 1.47E−01 | 4.02 | 3.69E−30 | 0.630 | |
| ENSG00000110931 | −0.08 | 8.06E−01 | 1.48 | 3.35E−24 | 0.501 | |
| ENSG00000027001 | −0.49 | 2.16E−01 | 0.88 | 2.86E−04 | 0.590 | |
| ENSG00000166743 | 0.20 | 7.86E−01 | 2.64 | 2.33E−09 | 0.570 | |
| ENSG00000260896 | −0.02 | 9.88E−01 | 3.33 | 3.67E−07 | 0.629 | |
| ENSG00000167900 | 0.53 | 3.18E−02 | 1.17 | 3.89E−11 | 0.495 | |
| ENSG00000130513 | −0.16 | 7.28E−01 | 1.85 | 1.44E−18 | 0.552 | |
| ENSG00000105707 | 0.30 | 3.53E−01 | 2.79 | 1.67E−46 | 0.560 | |
| ENSG00000130208 | 0.45 | 2.26E−01 | 2.03 | 1.79E−13 | 0.575 | |
| ENSG00000142515 | −0.31 | 2.40E−01 | 1.55 | 5.10E−13 | 0.512 | reference |
| | | | | | | gene |
| ENSG00000167751 | −0.35 | 1.34E−01 | 1.77 | 3.52E−17 | 0.607 | |
| ENSG00000167749 | −0.41 | 9.72E−02 | 1.42 | 6.67E−14 | 0.541 | |
| ENSG00000132821 | 0.39 | 3.49E−01 | 1.64 | 8.83E−11 | 0.606 | |
| ENSG00000170369 | 1.91 | 3.39E−03 | 2.81 | 1.86E−09 | 0.528 | |
| ENSG00000101210 | 0.46 | 3.99E−01 | 0.72 | 2.23E−02 | 0.557 | |
| ENSG00000157554 | 0.02 | 9.88E−01 | 1.52 | 3.79E−04 | 0.645 | |
| ENSG00000160180 | −1.00 | 9.84E−02 | 1.88 | 2.56E−06 | 0.640 | |
| ENSG00000280623 | −1.04 | 8.38E−02 | 3.00 | 6.86E−13 | 0.652 | |
| ENSG00000169083 | 0.21 | 1.64E−01 | 0.08 | 4.31E−01 | 0.513 | |
| ENSG00000158164 | 0.21 | 5.89E−01 | 1.91 | 8.30E−13 | 0.591 | |
| ENSG00000184012 | NA | NA | NA | NA | 0.692 | original |
| ENSG00000157554 | | | | | | MPS gene |

Multiplex qPCR OpenArray™ Profiling

OpenArray technology (Thermo Fisher Scientific™) was used for a high throughput real-time quantitative PCR (qPCR) method for rapid screening of multiple TaqMan assays. RNA isolation, extraction, and complementary DNA (cDNA) synthesis were performed (FIG. 3).

RNA isolation for the 54-gene OpenArray™ panel was performed using the MagMAX™ mirVana™ Total RNA Isolation Kit. Briefly, 500 microliters of a 1 to 1 mixture of urine and Hologic transport media were mixed 1 to 1 with Lysis Binding Mix™. Binding Beads Mix was added to enrich nucleic acids, followed by TURBO DNase™ digestion and RNA elution. For high-throughput RNA extraction, urine samples were processed using the semi-automatic KingFisher Flex System™ (Thermo Fisher Scientific™). All samples were run in triplicate.

After RNA extraction, 16 microliters of RNA were used to synthesize cDNA with SuperScript™ IV VILO™ Master Mix, followed by pre-amplification with TaqMan™ PreAmp Master Mix (Thermo Fisher Scientific™), according to the manufacturer's instructions. For each sample, 2.5 microliters of pro-amplified cDNA and 2.5 microliters of 2× TaqMan OpenArray™ Master Mix were loaded into 384-well plates per manufacturer instructions. The QuantStudio 12K Flex OpenArray™ AccuFill System transferred the mix to the TaqMan OpenArray™ plate. Amplification was performed using the QuantStudio™ 12K Flex Real Time PCR System, and the delta-delta cycle threshold method was used for analysis with the QuantStudio™ 12K Flex Software. In the development cohort, the OpenArray™ assay failed one or more replicates in 29 eligible controls and 25 eligible cases, yielding the final cohort for analysis. Normalized mean Ct values were used in determining the MPS2 algorithms as described in Examples 1 and 2 above.

### Specimen and Laboratory Analysis

Deidentified urine specimens were shipped to the University of Michigan for OpenArray profiling. A multiplex 2-gene algorithm (HOXC6 and DLX1) and a multiplex 3-gene algorithm (PCA, ERG, and SPDEF) were derived, and these genes were measured in SelectMDx and ExoDx Prostate Intelliscore (EPI) tests, respectively. Serum PSA, free PSA, and [−2]proPSA were measured using the Access 2 Immunoassay System (Beckman Coulter™).

### Results

RNA-sequencing analysis of 58,724 genes identified 54 markers prostate cancer, including 17 markers uniquely overexpressed by higher-grade cancers. Importantly, these 17 markers, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, are detectable in a subject's urine following a digital rectal examination (DRE), are uniquely overexpressed by GG≥2 prostate cancers, and are PSA-independent.

### Example 4

This Example describes development of an algorithm to assign a likelihood that a prostate biopsy of a subject would detect GG≥22 prostate cancer in men who are prostate biopsy-naïve or prostate biopsy-prior negative.

The RNA abundance of each of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 was measured in the urine of men directly prior (i.e., within about 60 minutes prior) to prostate biopsy. The urine was collected following a digital rectal exam. Additionally, the following clinical information was collected for each patient: age, PSA level, family history of prostate cancer (yes/no), African ancestry (yes/no) and DRE findings (normal/abnormal from any prior DRE). Following prostate biopsy, where applicable, the biopsy results were recorded and used for algorithm generation. Urinary RNA was measured following a protocol performed as described below. The protocol was followed for the RNA extraction, reverse transcription and pre-amplification PCR steps described below. RT-PCR was performed using a custom OpenArray® chip (ThermoFisher Scientific®) containing reaction wells for each of the genes, in triplicate. Generalized linear model with elastic net was used to build an algorithm to predict the risk of GG≥2 prostate cancer.

For qPCR, briefly, OpenArray technology (Thermo Fisher Scientific™) was used for a high throughput real-time quantitative PCR (qPCR) method for rapid screening of multiple TaqMan assays. RNA isolation, extraction, and complementary DNA (cDNA) synthesis were performed. KLK3 was used as the reference gene.

RNA isolation was performed using the MagMAX™ mirVana™ Total RNA Isolation Kit. Briefly, 500 microliters of a 1 to 1 mixture of urine and Hologic transport media were mixed 1 to 1 with Lysis Binding Mix™. Binding Beads Mix was added to enrich nucleic acids, followed by TURBO DNase™ digestion and RNA elution. For high-throughput RNA extraction, urine samples were processed using the semi-automatic KingFisher Flex System™ (Thermo Fisher Scientific™). All samples were run in triplicate.

After RNA extraction, 16 microliters of RNA were used to synthesize cDNA with SuperScript™ IV VILO™ Master Mix, followed by pre-amplification with TaqMan™ PreAmp Master Mix (Thermo Fisher Scientific™), according to the manufacturer's instructions. For each sample, 2.5 microliters of pre-amplified cDNA and 2.5 microliters of 2×TaqMan OpenArray™ Master Mix were loaded into 384-well plates per manufacturer instructions. The QuantStudio 12K Flex OpenArray™ AccuFill System transferred the mix to the TaqMan OpenArray™ plate. Amplification was performed using the QuantStudio™ 12K Flex Real Time PCR System, and the delta-delta cycle threshold method was used for analysis with the QuantStudio™ 12K Flex Software. Normalized mean Ct values were used.

Algorithms were built using the following inputs: Genes; Genes+Clinical Factor (without prostate volume); and Genes+Clinical Factor+Prostate Volume. The algorithm coefficients for each of the modeling approaches for prostate biopsy-prior negative and prostate biopsy-naïve men were derived using generalized linear modeling (Table C, Table E, and algorithm calibration intercept and slope values in Table G, above).

While various embodiments have been described herein, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as described herein.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

What is claimed is:

1. A method for assigning a likelihood that a prostate biopsy of a subject would detect Grade Group (GG)≥2 prostate cancer in the subject, the method comprising:
a) detecting an amount of expression of each of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample of the subject's urine;
b) normalizing the amount of expression of each of the genes to an amount of expression of a reference gene to provide a normalized target gene value for each of the genes;
c) multiplying each normalized target gene value by a corresponding gene algorithm coefficient to provide a gene log it value for each of the genes, wherein the corresponding gene algorithm coefficient is a corresponding gene algorithm coefficient of Table B or Table D;
d) summing 1) the gene log it values and 2) an algorithm intercept value to provide a sample log it value, wherein the algorithm intercept value is an algorithm intercept value of Table B or Table D;
e) multiplying the sample log it value by a calibration slope value to provide a product, and adding to the product a calibration intercept value to provide a calibrated log it value; and
f) obtaining a risk score comprising performing the equation
$$\frac{{e}^{calibrated logit value}}{1 + {e}^{calibrated logit value}}$$
wherein the risk score indicates the likelihood.

2. The method of claim 1, wherein the method further comprises comparing the risk score to a predetermined risk threshold value, and assigning a risk category based on whether the risk score is a) less than or equal to the risk threshold value, or b) higher than the risk threshold value.

3. The method of claim 2, wherein the risk category is a low-risk category where the risk score is a) less than or equal to the risk threshold value, and wherein the risk category is an increasing-risk category where the risk score is b) higher than the risk threshold value.

4. The method of any one of claims 1-3, wherein the subject is prostate biopsy-naïve.

5. The method of any one of claims 1-3, wherein the subject is prostate biopsy-prior negative.

6. The method of any one of claims 1-3, wherein the subject has had a prior positive prostate biopsy result for GG1 prostate cancer.

7. The method of any one of claims 1-6, wherein the sample is obtained within about 180 minutes after the subject has undergone a digital rectal exam (DRE).

8. The method of any one of claims 1-7, wherein the sample is obtained within about 60 minutes after the subject has undergone a DRE.

9. The method of any one of claims 1-6, wherein the sample is obtained from a subject who has not undergone a DRE.

10. The method of any one of claims 1-9, wherein the GG≥2 prostate cancer is GG≥3 prostate cancer.

11. The method of any one of claims 1-9, wherein the GG≥2 prostate cancer is GG≥4 prostate cancer.

12. The method of any one of claims 1-9, wherein the GG≥2 prostate cancer is GG5 prostate cancer.

13. The method of any one of claims 1-12, further comprising multiplying each of one or more clinical factor values by a corresponding clinical factor coefficient to provide a clinical factor log it value for each of the clinical factor values.

14. The method of claim 13, further comprising:
summing 1) the gene log it values, 2) an algorithm intercept value and 3) the clinical factor log it values to provide the sample log it value.

15. The method of claim 13 or 14, wherein the one or more clinical factor values are values of age, African ancestry, family history of prostate cancer, an abnormal DRE, PSA levels, prostate volume, or a combination thereof.

16. The method of any one of claims 1-15, wherein the gene algorithm coefficient is a gene algorithm coefficient of Table C or Table E, and wherein the algorithm intercept value is an algorithm intercept value of Table C or Table E.

17. The method of any one of claims 1-16, wherein the calibration slope value is a calibration slope value of Table F, and wherein the calibration intercept value is a calibration intercept value of Table F.

18. The method of any one of claims 1-17, wherein the calibration slope value is a calibration slope value of Table G, and wherein the calibration intercept value is a calibration intercept value of Table G.

19. The method of any one of claims 1-18, further comprising outputting to the subject or to a healthcare provider of the subject data relating to the risk score, risk threshold value, or risk category.

20. The method of any one of claims 1-19, further comprising generating a report comprising the risk score or risk category.

21. The method of claim 19, further comprising forwarding the report to the subject or to a healthcare provider of the subject.

22. The method of claim 20 or 21, wherein the subject's healthcare provider does not recommend a prostate biopsy of the subject where the subject's risk category is low-risk.

23. The method of claim 20 or 21, wherein where the subject's risk category is low-risk (ii) the subject does not undergo a prostate biopsy or (ii) the subject's healthcare provider does not recommend that the subject undergo a prostate biopsy.

24. The method of claim 20 or 21, wherein the subject's healthcare provider recommends a prostate biopsy where the subject's risk category is increasing-risk.

25. The method of claim 20 or 21, wherein where the subject's risk category is increasing-risk (i) the subject undergoes a prostate biopsy or (ii) the subject's healthcare provider recommends that the subject undergo a prostate biopsy.

26. The method of any one of claims 1-25, wherein the reference gene is KLK3, CYPB561A3, EEF1A2, GAPDH, HPN, KLK2, LBH, NUDT8, SPDEF, or TRGV.

27. The method of any one of claims 1-25, wherein the reference gene is KLK3.

28. The method of any one of claims 1-27, wherein the detecting the amount of expression is detecting an amount of mRNA expression.

29. The method of claim 28, wherein the detecting the amount of mRNA expression comprises synthesizing cDNA complementary to mRNA expressed by each of the genes, amplifying the cDNA, and detecting the cDNA for each gene.

30. A method for assigning a subject's Grade Group (GG)≥2 prostate cancer risk category, the method comprising:
a) detecting an amount of expression of each of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression of each of the genes is present in a sample of the subject's urine;
b) normalizing the amount of expression of each of the genes to an amount of expression of a reference gene to provide a normalized target gene value for each of the genes;
c) multiplying each normalized target gene value by a corresponding gene algorithm coefficient to provide a gene log it value for each of the genes, wherein the corresponding gene algorithm coefficient is a corresponding gene algorithm coefficient of Table B or Table D;
d) summing 1) the gene log it values and 2) an algorithm intercept value to provide a sample log it value, wherein the algorithm intercept value is an algorithm intercept value of Table B or Table D;
e) multiplying the sample log it value by a calibration slope value to provide a product, and adding to the product a calibration intercept value to provide a calibrated log it value;
f) obtaining a risk score comprising performing the equation
$$\frac{{e}^{calibrated logit value}}{1 + {e}^{calibrated logit value}}$$
g) comparing the risk score to a predetermined risk threshold value; and
h) assigning the risk category based on whether the risk score is 1) less than or equal to the risk threshold value, or 2) higher than the threshold value.

31. The method of claim 30, wherein the risk category is a low-risk category where the risk score is less than or equal to the risk threshold value, and wherein the risk category is an increasing-risk category where the risk score is higher than the risk threshold value.

32. The method of any one of claims 30-31, wherein the subject is prostate biopsy-naïve.

33. The method of any one of claims 30-32, wherein the subject is prostate biopsy-prior negative.

34. The method of any one of claims 30-33, wherein the subject has had a prior positive prostate biopsy result for GG1 prostate cancer.

35. The method of any one of claims 30-34, wherein the sample is obtained within about 180 minutes after the subject has undergone a digital rectal exam (DRE).

36. The method of any one of claims 30-35, wherein the sample is obtained within about 60 minutes after the subject has undergone a DRE.

37. The method of any one of claims 30-34, wherein the sample is obtained from a subject who has not undergone a DRE.

38. The method of any one of claims 30-36, wherein the GG≥22 prostate cancer is GG≥3 prostate cancer.

39. The method of any one of claims 30-36, wherein the GG≥2 prostate cancer is GG≥4 prostate cancer.

40. The method of any one of claims 30-38, wherein the GG≥2 prostate cancer is GG5 prostate cancer.

41. The method of any one of claims 30-40, further comprising multiplying each of one or more clinical factor values by a corresponding clinical factor coefficient to provide a clinical factor log it value for each of the clinical factor values.

42. The method of claim 41, further comprising:
summing 1) the gene log it values, 2) an algorithm intercept value and 3) the clinical factor log it values to provide the sample log it value.

43. The method of claim 41 or 42, wherein the one or more clinical factor values are values of age, African ancestry, family history of prostate cancer, an abnormal DRE, PSA levels, prostate volume, or a combination thereof.

44. The method of any one of claims 30-43, wherein the gene algorithm coefficient is a gene algorithm coefficient of Table C or Table E, and wherein the algorithm intercept value is an algorithm intercept value of Table C or Table E.

45. The method of any one of claims 30-43, wherein the calibration slope value is a calibration slope value of Table F, and wherein the calibration intercept value is a calibration intercept value of Table F.

46. The method of any one of claims 30-45, wherein the calibration slope value is a calibration slope value of Table G, and wherein the calibration intercept value is a calibration intercept value of Table G.

47. The method of any one of claims 30-46, further comprising outputting to the subject or to a healthcare provider of the subject data relating to the risk score, risk threshold value, or risk category.

48. The method of any one of claims 30-47, further comprising generating a report comprising the risk score, risk threshold value or risk category.

49. The method of claim 48, further comprising forwarding the report to the subject or to a healthcare provider of the subject.

50. The method of claim 48 or 49, wherein the subject's healthcare provider does not recommend a prostate biopsy where the subject's risk category is low-risk.

51. The method of claim 48 or 49, wherein where the subject's risk category is low-risk (i) the subject does not undergo a prostate biopsy or (ii) the subject's healthcare provider does not recommend that the subject undergo a prostate biopsy.

52. The method of claim 48 or 49, wherein the subject's healthcare provider recommends a prostate biopsy where the subject's risk category is increasing-risk.

53. The method of claim 48 or 49, wherein where the subject's risk category is increasing-risk (i) the subject undergoes a prostate biopsy or (ii) the subject's healthcare provider recommends that the subject undergo a prostate biopsy.

54. The method of any one of claims 30-53, wherein the reference gene is KLK3, CYPB561A3, EEF1A2, GAPDH, HPN, KLK2, LBH, NUDT8, SPDEF, or TRGV.

55. The method of any one of claims 30-53, wherein the reference gene is KLK3.

56. The method of any one of claims 30-55, wherein the detecting the amount of expression is detecting an amount of mRNA expression.

57. The method of claim 56, wherein the detecting the amount of mRNA expression comprises synthesizing cDNA complementary to mRNA expressed by each of the genes, amplifying the cDNA, and detecting the cDNA for each gene.


SEQUENCE LISTING (17 sequences)

SEQ ID NO: 1 - RNA, 293 nt, Homo sapiens
  source 1..293: mol_type = transcribed RNA
taggcgcgagctaagcaggaggcggaggcggaggcggagggcgaggggcggggagcgccgcctggagcgcggcaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaag

SEQ ID NO: 2 - RNA, 1574 nt, Homo sapiens
  source 1..1574: mol_type = transcribed RNA
gcttttatgagctgtaacactcaccgcgaaggtccgcagcttcactcctgaagccagcgagaccacgagcctactgggaggaacgaacaactcccgacgcgccgccttaagagctgtaacactcaccgcgaaggtctgcagcttcactcctgagccagcgagaccacgaacccaccagaaggaaaaaactccgaacacatctgaacatcagaagcaacaaactccggacacgccgcctttaagaactgtaacactcactgcgagggtccgcggcttcattcttgaagtgagtgagaccaagaacccaccagttctggacacaatttcaagtcctcaggtgccatcaatattctgaaaatggcagtgatttttattcaacctgtataaggcactttcaccatgtacctggaagcaacatctacatctttttcagtttcttctacgccaggtgtgtgcttagctccatgacaaaaggtgacagcttattctgcagcacacacacatcatcaaagtgggaggtggtgagactggcacactgacagtctgtcctagcagatttcagctcacactgcaatctagatgctggggacacaaggtccaccttccaggaatatggccatgacaccagaaatcacaaacatgatgagaatggaatgactggggaagaagtgccagatgcttcacttgtaaatgaagacccagcctctggggatgcagataccacctccctgaagaagctgaatatctgcagataagtggagttcaccaatgatgaggagcgggatggagaaaggaggtagggagagtcatccaaggaacatgagcaacatgttaaaagccaagtggtttaatttctggagatggtgaacccaagaggctctgctgggagacaacaaaaataatgaagaattgaaccagagtccggtgaatatcagcactgggaccagttagcagaggaaaaggaaagaataaaagcgaaaagaatgaagagtcatatgattaccaacttttcctttttcatataaattgagtgtatatgggtctggaacaacctgaatttccatcaagtcctggctaacctcattatgtcctatgaatatttttgactaatcccactttacattaatctgtattgtgaatgtggatattgaattatatttctttgtaatcccattatccaaaatccagttcagagactattagttaccaatgttcactgtgaaggaaaaaaaaaaaaaaaaagctcagaggataaacatgtgatatggtttggctgtgtccccacccaaatatcatcttgaattgtagctcccataattcccacgtgttgtgggagggacccggtgggagataattgtatcatgggggtggttcccccatactattctcatagtagtgaataagtctcacaaaatctgatggttttatgagggaaaacccctttcacctggttctcattctcttctctggtctgtcgtcatgtaagacatgcctttcaccttctccaccatgactgtgaggcctccccagccacgtggaactgtgagcccattaaacctctttcacttataaat

SEQ ID NO: 3 - RNA, 2792 nt, Homo sapiens
  source 1..2792: mol_type = transcribed RNA
cttctgggaatctccacaccctgaagacacagtgagttagcaccaccaccaggaattggcctttcagctctgtgcctgtctccagtcaggctggaataagtctcctcatatttgcaagctcggccctcccctggaatctaaagcctcctcagccttctgagtcagcctgaaaggaacaggccgaactgctgtatgggctctactgccagtgtgacctcaccctctccagtcacccctcctcagttccagctatgagttcctgcaacttcacacatgccacctttgtgcttattggtatcccaggattagagaaagcccatttctgggttggcttccccctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagtggtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaagtgacccttaacactacacttctccttatctttattggcttgataaacataattatttctaacactagcttatttccagttgcccataagcacatcagtacttttctctggctggaatagtaaactaaagtatggtacatctacctaaaggactattatgtggaataatacatactaatgaagtattacatgatttaaagactacaataaaaccaaacatgcttataacattaagaaaaacaataaagatacatgattgaaaccaagttgaaaaatagcatatgccttggaggaaatgtgctcaaattactaatgatttagtgttgtccctactttctctctcttttttctttcttttttttttattatggttagctgtcacatacaactttttttttttttgagatggggtctcgctctgtcaccaggctggagtgcagtggcgcgatctcggctcactgcaacctccacatcccatgttgaagtaattcttctgcctcagcctcccgagtagctgggactagaggaacgtgccaccatgactggctaattttctgtattttttagtagagacagagtttcaccatgttggccaggatggtctcgatctcctgaccttgtgatccacccgcctcagcctcccaaagtgttgggattacaggtgtgaaccactgtgcccggcctgtgtacaactttttaaatagggaatatgatagcttcgcatggtggtgtgcacctatagcccccactgcctggaaagctgaggtgggagaatcgcttgagtccaggagtttgaggttacagtgatccacgatcgtaccactacactccagcctgggcaacagagcaagaccctgtctcaaagcataaaatggaataacatatcaaatgaaacagggaaaatgaagctgacaatttatggaagccagggcttgtcacagtctctactgttattatgcattacctgggaatttatataagcccttaataataatgccaatgaacatctcatgtgtgctcacaatgttctggcactattataagtgcttcacaggttttatgtgttcttcgtaactttatggagtaggtaccatttgtgtctctttattataagtgagagaaatgaagtttatattatcaaggggactaaagtcacacggcttgtgggcactgtgccaagatttaaaattaaatttgatggttgaatacagttacttaatgaccatgttatattgcttcctgtgtaacatctgccatttatttcctcagctgtacaaatcctctgttttctctctgttacacactaacatcaatggctttgtacttgtgatgagagataaccttgccctagttgtgggcaacacatgcagaataatcctgttttacagctgcctttcgtgatcttattgcttgcttttttccagattcagggagaatgttgttgtctatttgtctcttacatctccttgatcatgtcttcattttttaatgtgctctgtacctgtcaaaaattttgaatgtacaccacatgctattgtctgaacttgagtataagataaaataaaattttattttaaatttt

SEQ ID NO: 4 - RNA, 514 nt, Homo sapiens
  source 1..514: mol_type = transcribed RNA
aggcggtcaggggaaggctcaggaggagggagatcaacatcaacctgccccgccccctccccagcctgataaaggtcctgcgggcaggacaggacctcccaaccaagccctccagcaaggattcagagtgcccctccggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaa

SEQ ID NO: 5 - RNA, 1201 nt, Homo sapiens
  source 1..1201: mol_type = transcribed RNA
gagatacggcctcgtgggaagggaaagacctgaccgtcccccagcccgacacccgtaaagggtctgtgctgaggaggattagtaaaaggggaaggcctcttgcagttgagataagaggaaggcctccgtctcctgcatgtccttgggaatggaatgtcttggtgtaaaacccgatagtacattccttctattctgagagaagaaaaccaccctgtggctggagggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaatt

SEQ ID NO: 6 - RNA, 4905 nt, Homo sapiens
  source 1..4905: mol_type = transcribed RNA
agagcaagctgagccgagccgagccgagctgggggcgcagagcgcgggaggcggcggcggcgcggagcccaggtggctccgctgccggatgggagtgccccagtgtgctggatgaagctggcgcatgcaccatgtcatcatgtgtctctagccagcccagcagcaaccgggccgccccccaggatgagctggggggcaggggcagcagcagcagcgaaagccagaagccctgtgaggccctgcggggcctctcatccttgagcatccacctgggcatggagtccttcattgtggtcaccgagtgtgagccgggctgtgctgtggacctcggcttggcgcgggaccggcccctggaggccgatggccaagaggtcccccttgacacctccgggtcccaggcccggccccacctctccggtcgcaagctgtctctgcaagagcggtcccagggtgggctggcagccggtggcagcctggacatgaacggacgctgcatctgcccgtccctgccctactcacccgtcagctccccgcagtcctcgcctcggctgccccggcggccgacagtggagtctcaccacgtctccatcacgggtatgcaggactgtgtgcagctgaatcagtataccctgaaggatgaaattggaaagggctcctatggtgtcgtcaagttggcctacaatgaaaatgacaatacctactatgcaatgaaggtgctgtccaaaaagaagctgatccggcaggccggctttccacgtcgccctccaccccgaggcacccggccagctcctggaggctgcatccagcccaggggccccattgagcaggtgtaccaggaaattgccatcctcaagaagctggaccaccccaatgtggtgaagctggtggaggtcctggatgaccccaatgaggaccatctgtacatggtgttcgaactggtcaaccaagggcccgtgatggaagtgcccaccctcaaaccactctctgaagaccaggcccgtttctacttccaggatctgatcaaaggcatcgagtacttacactaccagaagatcatccaccgtgacatcaaaccttccaacctcctggtcggagaagatgggcacatcaagatcgctgactttggtgtgagcaatgaattcaagggcagtgacgcgctcctctccaacaccgtgggcacgcccgccttcatggcacccgagtcgctctctgagacccgcaagatcttctctgggaaggccttggatgtttgggccatgggtgtgacactatactgctttgtctttggccagtgcccattcatggacgagcggatcatgtgtttacacagtaagatcaagagtcaggccctggaatttccagaccagcccgacatagctgaggacttgaaggacctgatcacccgtatgctggacaagaaccccgagtcgaggatcgtggtgccggaaatcaagctgcacccctgggtcacgaggcatggggcggagccgttgccgtcggaggatgagaactgcacgctggtcgaagtgactgaagaggaggtcgagaactcagtcaaacacattcccagcttggcaaccgtgatcctggtgaagaccatgatacgtaaacgctcctttgggaacccattcgagggcagccggcgggaggaacgctcactgtcagcgcctggaaacttgctcaccaaaaaaccaaccagggaatgtgagtccctgtctgagctcaaggaagcaaggcagcgaagacaacctccagggcaccgacccgccccccgtgggggaggaggaagtgctcttgtgagaggcagtccctgcgtggaaagttgctgggcccccgcccccggctcccccgcacgcatgcatccactgcggccggaggaggccatggagcccgagtagctgcctggatcgctcgacctcgcatgcgcgccgcgtcgcctctggggggctgctgcaccgcgtttccatagcagcatgtcctacggaaacccagcacgtgtgtagagcctcgatcgtcatctctggttatttgttttttcctttgttgttttaaaggggacaaaaaaaaaaaaaggacttgactccatgacgtcgaccgtggccgctggctggctggacaggcgggtgtgaggagttgcagacccaaacccacgtgcattttgggacaattgctttttaaaacgtttttatgccaaaaatccttcattgtgattttcagaaccacgtcagatataccaagtgactgtgtgtggggtttgacaactgtggaaaggcgagcagaaaactccggcggtctgaggccatggaggtggttgctgcatttgagagggagtagggggctagatgtggctcctagtgcaaaccggaaaccatggcaccttccagagccgtggtctcaaggagtcagagcagggctggccctcagtagctgcagggagctttgatgcaacttatttgtaagaaggatttttaaattttttatgggtagaattgtagtcaggaaaacagaaagggcttgaaatttaataagtgctgctggaaggggattttccaagcctggaagggtattcagcagctgtggtggggaaacatttctcctgaaagactgaacgtgtttcttcatgacagctgctcaaagcaggtttctgagatagctgaccgagctctggtaaatctctttgtcaaattacgaaaacttcagggtgaaatcctatgcttccatgtacattacatggcttaagattaaacaaaaacatttttcaagtctctaactagagtgaactctagagcacagtagttcagaaactatttagagcttccaggatatatttcacagcttcaggcatgtgatcagttagagccgatgaaacctatgcccgcctgtatatatattagcagcttagctagttcataacctgtatattctaaagactgctaaggttttgttttcattttaaatcctagctgattgttgtggtcaatgaaatacccagtttctggagggccaggtgggaaatgctttcactggaccaacacacaaatgatcatcctgaggatctgagcttccctagactccacacaataaccttggggcacccttttagagaagactgttgaaacccacagcactcgttggggtatgaggaaaccagggcttggcacaggaagttcccctttgtagctaaaagtccagaaagaaagggttcatctttttgacttccaactgatattgggaagtttggttgaggttcaagtgtgactccttccagagccacaggtaggggagtgtgaagttgagggggaggaaagctggaaggactctgccttgggagattcccagctctgctttccagcgcttggtggaatctgggctggggaaagacggcaccgggaaactctgcttccccattgtttccatctgatcagctgtggtgtgaggacttctcagacaaaggcaaggcctcgtgcccctgcccagcccattcatggagccctgggccttcttggcttccatagatcctaagctcttgactgtagtttagccagacttgttttgctatcttataagcagttcagaattagggaatgctggttttgaagagcaaaggacaggtagtctagagagggtcgtctggcctgcttgctgggtctttgtaacccagcacttcctcttgccctcctggctttatgtttatggggagaggactcaatagctccaccccttctggcaccagatggggcttggttagtttgcaataagcaccttgcagaggttaaagccagcgggtccctagtcttaggcccagcctgcttgtgtgggctctggcctggcctggtggctggcccagggggcagcagtgcttagagcttctgcagggcttctcttgtttacacagctgcatcagacaatgccatttctccccaccacggaaccttccatctaagatttcttccagggaatgccagcaatcaggcagcacccagctgtgggggcagtggggtgggggagacccacattgatgactttttttttttcttttaatgaagaaacaccaaagaaagctgtggaaaggacctgccccacatgaaaaggataagccaagatggctgtaaacacagagcatttgagctgccactcttggagcacattgatttttcaaaagccagctctgtcaggaaaggaggtgctgttatgagcagctcttccagtgggcaaagaggacgcccataatttcttccattgctagctcatctgtgggaccaatttggtgtaagcaacctgtggcctgcacttgtggcctcgaaggaagcacaaaccctccatccacttcccatttcctctgcccttttccacctcccccttccatcccaccagctgccagtggctcccagaaagccttattgagccccttgttgacacttggggctgcggaggcctctccctactggtctggcctttcctgagaggcaggtcttccgtcctcagagcctttctggaacaaggagaatgcctgtgcaggtggacacacaggcctggcctgtcgctctcacttgtcttccagcggggagcttcacgttgccgagtggaagaaccatgacctccacttgcttccaaggtgctagggaagtttcagggtacgctggttcccctctccagctggaggccgagtttctggggactgcagatttttctactctgtgatcgattcaatgcccgatgcttctgtttcattcccgaccctttctactatgcattttccttttatcaggtgtataaagttaaatactgtgtatttatcactaaaaagtacatgaacttaagagacaactaagcctttcgtgtttttccacaggtgtttaagcttctctgtacagttgaaataaacagacagcaaaatggtgccaa

SEQ ID NO: 7 - RNA, 3757 nt, Homo sapiens
  source 1..3757: mol_type = transcribed RNA
acagaagaaatagcaagtgccgagaagctggcatcagaaaaacagaggggagatttgtgtggctgcagccgagggagaccaggaagatctgcatggtgggaaggacctgatgatacagaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagccacagcctctccccatccctccagccttatctgtcatcaccatcaacccctcccatgcacctaaacaaaatctaacttgtaattccttgaacatgtcaggcatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgagtgtacatgccaaagtgtgcctctctctctttgacccattatttcagacttaaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 8 - RNA, 2922 nt, Homo sapiens
  source 1..2922: mol_type = transcribed RNA
agtgctgctctgcgctgcgccgcgctcggggctcgctctccttgctccgcgctccccgccagccgccccggggcaggaggcgcgcctgacggacggcccgctagacaaaggaggcgcggctcggcggggccagcgcgcggacggacggaccatggactcggagcgcgggcggccggccccagccttggggaccggacactcccgggcccggccctaggcgcccggccccgccgcccggcgcgcccagcggggaggacgtggagcccgcgcggcgcgagcaggcggcggccgcggagcaagaagggcgccgcggcgtgcggcccgcgcagcccccggagccatgggcaagtgcagcgggcgctgcacgctggtcgccttctgctgcctgcagctggtggctgcgctggagcggcagatctttgacttcctgggctaccagtgggctcccatcctagccaacttcctgcacatcatggcagtcatcctgggcatctttggcaccgtgcagtaccgctcccggtacctcatcctgtatgcagcctggctggtgctctgggttggctggaatgcatttatcatctgcttctacttggaggttggacagctgtcccaggaccgggacttcatcatgaccttcaacacatccctgcaccgctcctggtggatggagaatgggccaggctgcctggtgacacctgttctgaactcccgcctggctctggaggaccaccatgtcatctctgtcactggctgcctgcttgactacccctacattgaagccctcagcagcgccctgcagatcttcctggcactgttcggcttcgtgttcgcctgctacgtgagcaaagtgttcctggaggaggaggacagctttgacttcatcggcggctttgactcctacggataccaggcgccccagaagacgtcgcatttacagctgcagcctctgtacacgtcggggtagcctctgccccgcgcccaccccggcgcctcgccctgggctgaccgcagctgccgcgagctcgggccaaggcgcaggcgtgtccccctggtggcccgcgcgctcactgcagcctgtgcccaaccccgcgtctgcatctggagatgcggacttggacgtggacttggacttggacttggatttgagcttggctcttcgcagcccggacttcggaggagtggggcggggcgggggaggggcaccacgggttttttgttttttgtttgtttgtttttaatctcagccttggcgtgagctggggccttcctctcttctccagcctctccctttcactcttcacccagcatcctgcccccctgtccaaaaacagcaggacatcagacccatcccatcccaccacactcactcaccagctctggggaaagctactgtgaactaggagcaggattcctgggttctaatcgcaggtccatcactgactgtgacgtctagcaaagcccttgccctctctgagcctcggtttccgcacctcaagtaattaatcccttagcaaatggactcttttagacttctcatttaactcaattccctgagctagactgggattaaaattctcattttgcagtacattaaaactgaggcccagagatgtgatttgcttgaggccacacagctagatttttggtggaagtgggccttgaacacagtgtactttctgcagtttctgactgtaaaacccagtgtctgctctctgagttccatttccaagcccccctccatcttggacctatgtggtctccaccatattcacacaccaccaccaccacttgccaatgcctctcttaaagcaatatacccattcgttctcttattgggaactggatggatgaagccccaaattcagccccacccacagagaagccttcctacactcagcctctgtccacccttggcaaatctttcaagctctctcctccaggaaagtggggccccaactcagtcactccacccccttccaggtccctgaggctggttctactgtatccccatcacctccacaactccactcacccctgacggctccatccacctcaccagttggaaggcttgtggtttcagagaggagcaatgctggtcagcgctgcccagactccagtgtttacagatcaccagcatttacaaccaatccaatggccagaagcctcctctaacaagcccagaaggagttctgaaggggcagatgggggtgtgagtagtcggggagtcgggattgccagcaccctcacccttccttgggggcaagtagaggtgagaacactttccccacctccctccacagacactcctgaggacgctgcatcccacgcactgcctggtgcgtccatagagagaggatcaggtctcagcatttcatctgtgaaagaggcatggccctgggttagaaaggagggcaggagacatggaggaactggggggcacccagatggtgcagatggtttgcacacctgagcctgtctgtggtgaccattccgctcctctcccactaccctccaatctatcattccctactctctaaggccaaaatatcctgagcaaggctggcaaccccaccccaccatcccaaatgcaagcagccaggcccaggagttcctctggcccccacaggcatggagctcccagctggtgggtacagcttgagaggggggcagctccctcaggctaagctactgcccttcactgggccagccctgcctccagccctcacctctctcaccccaactctcccccaagcccctttctactcaacgggtgtagccactggtgctttgaagccttttgtttttataagatggtttttgcaaggggaccaggttctcttttcactgggaccttgcaaggaggggagtgctctcctggtttctgtgcaggcgggttgattaaagatggtgttttcttctcta

SEQ ID NO: 9 - RNA, 2503 nt, Homo sapiens
  source 1..2503: mol_type = transcribed RNA
agtgtgtgaagtaaagggattaaaggctagtctcaggctggggatggctcctgtctatttcttctctctcagagactgcagatggcttttccctgccgcaggtccctgactgccaagactctggcctgcctcctggtgggcgtgagtttcttagcactgcagcagtggttcctccaggcgccaaggtccccgcgggaggagaggtccccgcaggaggagacgccagagggtcccaccgacgctcccgcggctgacgagccgccctcggagctcgtccccgggcccccgtgcgtggcgaacgcctcggcgaacgccacggccgacttcgagcagctgcccgcgcgcatccaggacttcctgcggtaccgccactgccgccacttcccgctgctttgggacgcaccggccaagtgcgccggcggccgaggcgtgttcctgctcctggcggtgaagtcggcgcctgagcactacgagcgacgcgagctcatccggcgcacgtgggggcaagagcgcagctacggcgggcggccagtgcgccgcctctttctattgggcaccccgggccccgaggacgaggcgcgcgcggagcggctggcggagctggtggcgctggaggcgcgcgagcacggcgacgtgctgcagtgggccttcgcggacaccttcctcaacctcacgctcaagcacctgcacttgctcgactggctggctgcacgctgcccgcacgcgcgctttctgctcagcggcgacgacgacgtgttcgtgcacaccgccaacgtagtccgcttcctgcaggcgcagccacccggccgccacctgttctccggccagctcatggagggctccgtgcccatccgcgacagctggagcaagtacttcgtgccgccgcagctcttccccgggtccgcttacccggtgtactgcagcggcggcggcttcctcctgtccggccccacggcccgggccctgcgcgcggccgcccgccacaccccgctcttccccatcgacgacgcctacatgggcatgtgtctggagcgcgccggcctggcgcccagcggccacgagggcatccgacccttcggcgtgcagctgcctggcgcacagcagtcctccttcgacccctgcatgtaccgcgagttgctgctagtgcaccgcttcgcgccctacgagatgctgctcatgtggaaggcgctgcacagccccgcgctcagctgtgaccggggacaccgggtctcctgaggccagttgggcggcttcagccccgggcctccaaccatgtccatgctgagaaggcagctttcccgctctgggtaccttacgtcctgcccagctctgtgcacctgaaccccagctgcgcactgaaatcagctggggtggggggtgtggaaaatgcctacatcctggctccatctcccgaagtttcgatttgattagtctggggtggacccagacatgttaagtattttttaagttcctccagtgatgcgaatgtgcagctaggcctgaggaccactcggctagactatctcttcatcctcgcaaagccagctccaccgccctctctgcaagaattccgggcccctcgctcccacactcgggtcctcttgagcagtggagcaagggagacctgggagcgtgggagccaggatcagcgccccctgccatgtgcctacaaatgtcagttgtgatttccactgtttacaagtgagtggagctggagctgggctgacagtatcaggtggatcccgcttccccctcccccaagaagtcagccaacacgcagctgaggcgcatgtggtggccttcttcccaccactaccccagtacaccgtgaggtagaaatcttcaccgtgcaaagtggaaaccagaggcccggtcagacagtgactaatccagggccgtggcattcccagacagcacaccactgtggtcccctccacactcaccccaaccaaagctaatggcctagttgggtcctgcccgccaataatcacccccacgggtcagagacaggctccttgccggggtctgggcctcaggctcagtgggccttggacaacccagcagggagttccggggagtccgaagtggagaaaggctggtgggaacatggaggccagtgttggggagcctgtggaggcaggtgtgtagaattgtgttcgggaggtgggggatctgagaccgaagtggacagtggttaagattgtggggccgggcgaggtggctcacgcctgtaatcccagcactttgggaggctgaggaggtcggatcatgaggtcaagagttcgagaccagcctggccaatatggtgaaaccccgtctctattgggagtacaaaaattagccggccatagtggctcgtgcctgtaatctcagctatttgggaggctgaggcaggagaatcacttgaacctgggaggcggaggttgcagtgagccgagatcgtgccactgcactccagcctgggcgacagagcaagactgcatctcaaaaaaaaaaaaaaaaa

SEQ ID NO: 10 - RNA, 867 nt, Homo sapiens
  source 1..867: mol_type = transcribed RNA
gagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgagactttctgaggttgtgctttatttctgctgcgtcgtgggagagggcgggagggtgtcaggggagagtctgcccaggcctcaagggcaggaaaagactccctaaggagctgcagtgcatgcaaggatattttgaatccagactggcacccacgtcacaggaaagcctaggaacactgtaagtgccgcttcctcgggaaagcagaaaaaatacatttcaggtagaagttttcaaaaatcacaagtctttcttggtgaagacagcaagccaataaaactgtcttccaaagtggtcctttatttcacaaccactctcgctactgttcaatacttgtactattcctgggttttgtttctttgtacagtaaacattatgaacaaacaggca

SEQ ID NO: 11 - RNA, 1579 nt, Homo sapiens
  source 1..1579: mol_type = transcribed RNA
acccgaccgctgccggccgcgctcccgctgctcctgccgggtgatggaaaaccccagcccggccgccgccctgggcaaggccctctgcgctctcctcctggccactctcggcgccgccggccagcctcttgggggagagtccatctgttccgccagagccctggccaaatacagcatcaccttcacgggcaagtggagccagacggccttccccaagcagtaccccctgttccgcccccctgcgcagtggtcttcgctgctgggggccgcgcatagctccgactacagcatgtggaggaagaaccagtacgtcagtaacgggctgcgcgactttgcggagcgcggcgaggcctgggcgctgatgaaggagatcgaggcggcgggggaggcgctgcagagcgtgcacgcggtgttttcggcgcccgccgtccccagcggcaccgggcagacgtcggcggagctggaggtgcagcgcaggcactcgctggtctcgtttgtggtgcgcatcgtgcccagccccgactggttcgtgggcgtggacagcctggacctgtgcgacggggaccgttggcgggaacaggcggcgctggacctgtacccctacgacgccgggacggacagcggcttcaccttctcctcccccaacttcgccaccatcccgcaggacacggtgaccgagataacgtcctcctctcccagccacccggccaactccttctactacccgcggctgaaggccctgcctcccatcgccagggtgacactggtgcggctgcgacagagccccagggccttcatccctcccgccccagtcctgcccagcagggacaatgagattgtagacagcgcctcagttccagaaacgccgctggactgcgaggtctccctgtggtcgtcctggggactgtgcggaggccactgtgggaggctcgggaccaagagcaggactcgctacgtccgggtccagcccgccaacaacgggagcccctgccccgagctcgaagaagaggctgagtgcgtccctgataactgcgtctaagaccagagccccgcagcccctggggccccccggagccatggggtgtcgggggctcctgtgcaggctcatgctgcaggcggccgagggcacagggggtttcgcgctgctcctgaccgcggtgaggccgcgccgaccatctctgcactgaagggccctctggtggccggcacgggcattgggaaacagcctcctcctttcccaaccttgcttcttaggggcccccgtgtcccgtctgctctcagcctcctcctcctgcaggataaagtcatccccaaggctccagctactctaaattatgtctccttataagttattgctgctccaggagattgtccttcatcgtccaggggcctggctcccacgtggttgcagatacctcagacctggtgctctaggctgtgctgagcccactctcccgagggcgcatccaagcgggggccacttgagaagtgaataaatggggcggtttcggaagcgtcagtgtttccatgttatggatctctctgcgtttgaataaagactatctctgttgctcacaaa

SEQ ID NO: 12 - RNA, 1603 nt, Homo sapiens
  source 1..1603: mol_type = transcribed RNA
aaggcactctggcacccagttttggaactgcagttttaaaagtcataaattgaatgaaaatgatagcaaaggtggaggtttttaaagagctatttataggtccctggacagcatcttttttcaattaggcagcaacctttttgccctatgccgtaacctgtgtctgcaacttcctctaattgggaaatagttaagcagattcatagagctgaatgataaaattgtactacgagatgcactgggactcaacgtgaccttatcaagtgagcaggcttggtgcatttgacacttcatgatatcagccaaagtggaactaaaaacagctcctggaagaggactatgacatcatcaggttgggagtctccagggacagcggaccctttggaaaaggactagaaagtgtgaaatctattagtcttcgatatgaaattctctgtctctgtaaaagcatttcatatttacaagacacaggcctactcctagggcagcaaaaagtggcaacaggcaagcagagggaaaagagatcatgaggcatttcagagtgcactgtcttttcatatatttctcaatgccgtatgtttggttttattttggccaagcataacaatctgctcaagaaaaaaaaatctggagaaaacaaaggtgcctttgccaatgttatgtttctttttgacaagccctgagatttctgaggggaattcacataaatgggatcaggtcattcatttacgttgtgtgcaaatatgatttaaagatacaacctttgcagagagcatgctttcctaagggtaggcacgtggaggactaagggtaaagcattcttcaagatcagttaatcaagaaaggtgctctttgcattctgaaatgcccttgttgcaaatattggttatattgattaaatttacacttaatggaaacaacctttaacttacagatgaacaaacccacaaaagcaaaaaatcaaaagccctacctatgatttcatattttctgtgtaactggattaaaggattcctgcttgcttttgggcataaatgataatggaatatttccaggtattgtttaaaatgagggcccatctacaaattcttagcaatactttggataattctaaaattcagctggacattgtctaattgttttttatatacatctttgctagaatttcaaattttaagtatgtgaatttagttaattagctgtgctgatcaattcaaaaacattactttcctaaattttagactatgaaggtcataaattcaacaaatatatctacacatacaattatagattgtttttcattataatgtcttcatcttaacagaattgtctttgtgattgtttttagaaaactgagagttttaattcataattacttgatcaaaaaattgtgggaacaatccagcattaattgtatgtgattgtttttatgtacataaggagtcttaagcttggtgccttgaagtcttttgtacttagtcccatgtttaaaattactactttatatctaaagcatttatgtttttcaattcaatttacatgatgctaattatggcaattataacaaatattaaagatttcgaaatagaaaaaaaaaaaaaaa

SEQ ID NO: 13 - RNA, 1530 nt, Homo sapiens
  source 1..1530: mol_type = transcribed RNA
gtgaggacaccgctttacaacgatgcagggggccccatgtcaccctcacccatgggaagtttgacttggtggactcagccaagccacagaggtctaacgcttctctgcggtgatttcaggctgccctggcagaaagcacagtgcctgcagacatgctgtcactgctccacgcatcaacgctggcagtccttggggctctgtgtgtatatggtgcaggtcacctagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgttggagggaaattataagaaactctttggcagtggaacaacacttgttgtcacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagctcacaaacacctctgcatattacatgtacctcctcctgctcctcaagagtgtggtctattttgccatcatcacctgctgtctgcttagaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataacggttttcaaaccagtgggcacacagagaacctcactctgtaataacaatgaggaatagccacggcgatctccagcaccaatctctccatgttttccacagctcctccagccaacccaaatagcgcctgctatagtgtagacatcctgcggcttctagccttgtccctctcttagtgttctttaatcagataactgcctggaagcctttcattttacacgccctgaagcagtcttctttgctagttgaattatgtggtgtgtttttccgtaataagcaaaataaatttaaaaaaatgaaaagtt

SEQ ID NO: 14 - RNA, 634 nt, Homo sapiens
  source 1..634: mol_type = transcribed RNA
aacgctaacctggtccggagcgagtctgggtctcagccccgcgaacagcctttcacgagtcttcaagctttcaggctatcttctagtcaagatgagtgataagccagacttgtcggaagtggagaagtttgacaggtcaaaactgaagaaaactaatactgaagaaaaaaatactcttccctcaaaggaaactatccagcaagagaaagagtgtgttcaaacatcataaaatggggatcgcctcccaacagcagatttcgacattacctgagagtcttgattttaggcttgttttttgtaaacccatgtgtttgtagagattttaggcgtcttcggatatcttctcacctatgttccctggctaagaagtcagaggtagccaatgtttccttaaattcatttttaaacttaccattggtgcatatgttccagatggcagatgctgtcaataatctcaccattgatgacctttgtgtatgtagttcttgcatcctatactggataagcctgttttaacctgctatgatgggtgcttccattgcttcataatcttcatgaagttgcatgcttttgcagcttttcacagtttatttgcatttctaatgtagtaataaagtaaccaatataatcatta

SEQ ID NO: 15 - RNA, 4904 nt, Homo sapiens
  source 1..4904: mol_type = transcribed RNA
atccgctctaaacaacctcatcaaaactactttctggtcagagagaagcaataattattattaacatttattaacgatcaataaacttgatcgcattatggccagcactattaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttggaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcaaataaagcgtcatggatagctgcaatttctcactttacagaaacaagggataacgtctagatttgctgcggggtttctctttcaggagctctcactaggtagacagctttagtcctgctacatcagagttacctgggcactgtggcttgggattcactagccctgagcctgatgttgctggctatcccttgaagacaatgtttatttccataatctagagtcagtttccctgggcatcttttctttgaatcacaaatgctgccaaccttggtccaggtgaaggcaactcaaaaggtgaaaatacaaggtgaccgtgcgaaggcgctagccgaaacatcttagctgaataggtttctgaactggcccttttcatagctgtttcagggcctgtttttttcacgttgcagtccttttgctatgattatgtgaagttgccaaacctctgtgctgtggatgttttggcagtgggctttgaagtcggcaggacacgattaccaatgctcctgacaccccgtgtcatttggattagacggagcccaaccatccatcattttgcagcagcctgggaaggcccacaaagtgcccgtatctccttagggaaaataaataaatacaatcatgaaagctggcagttaggctgacccaaactgtgctaatggaaaagatcagtcatttttattttggaatgcaaagtcaagacacacctacattcttcatagaaatacacatttacttggataatcactcagttctctcttcaagactgtctcatgagcaagatcataaaaacaagacatgattatcatattcaattttaacagatgttttccattagatccctcaaccctccacccccagtccaggttattagcaagtcttatgagcaactgggataattttggataacatgataatactgagttccttcaaatacataattcttaaattgtttcaaaatggcattaactctctgttactgttgtaatctaattccaaagccccctccaggtcatattcataattgcatgaaccttttctctctgtttgtccctgtctcttggcttgccctgatgtatactcagactcctgtacaatcttactcctgctggcaagagatttgtcttcttttcttgtcttcaattggctttcgggccttgtatgtggtaaaatcaccaaatcacagtcaagactgtgtttttgttcctagtttgatgcccttatgtcccggaggggttcacaaagtgctttgtcaggactgctgcagttagaaggctcactgcttctcctaagccttctgcacagatgtggcacctgcaacccaggagcaggagccggaggagctgccctctgacagcaggtgcagcagagatggctacagctcaggagctgggaaggtgatggggcacagggaaagcacagatgttctgcagcgccccaaagtgacccattgcctggagaaagagaagaaaatattttttaaaaagctagtttatttagcttctcattaattcattcaaataaagtcgtgaggtgactaattagagaataaaaattactttggactactcaaaaa

SEQ ID NO: 16 - RNA, 1416 nt, Homo sapiens
  source 1..1416: mol_type = transcribed RNA
aggcagcaggctggagctcagcccagcagtggaatccaggagcccagaggtggccgggtgctgacgtgatggccacagcaggaaatccctggggctggttcctggggtacctcatccttggtgtcgcaggatcgctcgtctctggtagctgcagccaaatcataaacggcgaggactgcagcccgcactcgcagccctggcaggcggcactggtcatggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaactctggggactgggaacccatgaaattgacccccaaatacatcctgcggaaggaattcaggaatatctgttcccagcccctcctccctcaggcccaggagtccaggcccccagcccctcctccctcaaaccaagggtacagatccccagcccctcctccctcagacccaggagtccagaccccccagcccctcctccctcagacccaggagtccagcccctcctccctcagacccaggagtccagaccccccagcccctcctccctcagacccaggagtccagcccctcctccctcagacccaggagtccagaccccccagcccctcctccctcagacccaggggtccaggcccccaacccctcctccctcagactcagaggtccaggcccccaacccctccttccccagacccagaggtccaggtcccagcccctcctccctcagacccagcggtccaatgccacctagactctccctgtacacagtgcccccttgtggcacgttgacccaaccttaccagttggtttttcattttttgtccctttcccctagatccagaaataaagtctaagagaagcgca

SEQ ID NO: 17 - RNA, 1651 nt, Homo sapiens
  source 1..1651: mol_type = transcribed RNA
ataaccatctagttccgagtacaaactggagacagaaataaatattaaagaaatcatagaccgaccaggtaaaggcaaagggatgaattcctacttcactaacccttccttatcctgccacctcgccgggggccaggacgtcctccccaacgtcgccctcaattccaccgcctatgatccagtgaggcatttctcgacctatggagcggccgttgcccagaaccggatctactcgactcccttttattcgccacaggagaatgtcgtgttcagttccagccgggggccgtatgactatggatctaattccttttaccaggagaaagacatgctctcaaactgcagacaaaacaccttaggacataacacacagacctcaatcgctcaggattttagttctgagcagggcaggactgcgccccaggaccagaaagccagtatccagatttacccctggatgcagcgaatgaattcgcacagtggggtcggctacggagcggaccggaggcgcggccgccagatctactcgcggtaccagaccctggaactggagaaggaatttcacttcaatcgctacctaacgcggcgccggcgcatcgagatcgccaacgcgctttgcctgaccgagcgacagatcaaaatctggttccagaaccgccggatgaagtggaaaaaagaatctaatctcacatccactctctcggggggcggcggaggggccaccgccgacagcctgggcggaaaagaggaaaagcgggaagagacagaagaggagaagcagaaagagtgaccaggactgtccctgccacccctctctccctttctccctcgctccccaccaactctcccctaatcacacactctgtatttatcactggcacaattgatgtgttttgattccctaaaacaaaattagggagtcaaacgtggacctgaaagtcagctctggaccccctccctcaccgcacaactctctttcaccacgcgcctcctcctcctcgctcccttgctagctcgttctcggcttgtctacaggcccttttccccgtccaggccttgggggctcggaccctgaactcagactctacagattgccctccaagtgaggacttggctcccccactccttcgacgcccccacccccgccccccgtgcagagagccggctcctgggcctgctggggcctctgctccagggcctcagggcccggcctggcagccggggagggccggaggcccaaggagggcgcgccttggccccacaccaacccccagggcctccccgcagtccctgcctagcccctctgccccagcaaatgcccagcccaggcaaattgtatttaaagaatcctgggggtcattatggcattttacaaactgtgaccgtttctgtgtgaagatttttagctgtatttgtggtctctgtatttatatttatgtttagcaccgtcagtgttcctatccaatttcaaaaaaggaaaaaaaagagggaaaattacaaaaagagagaaaaaaagtgaatgacgtttgtttagccagtaggagaaaataaataaataaataaatcccttcgtgttaccctcctgtataaatccaacctctgggtccgttctcgaatatttaataaaactgatattatttttaaaacttta